SEQUENCE LISTING (112 sequences)

SEQ ID NO: 1 - protein, 4 aa, synthetic construct
DYYI

SEQ ID NO: 2 - protein, 17 aa, synthetic construct
WINPNSGDTNYAQKFQG

SEQ ID NO: 3 - protein, 9 aa, synthetic construct
NSGSYSFGY

SEQ ID NO: 4 - protein, 11 aa, synthetic construct
RASQGISSWLA

SEQ ID NO: 5 - protein, 7 aa, synthetic construct
GASSLQS

SEQ ID NO: 6 - protein, 9 aa, synthetic construct
QQAYSFPFT

SEQ ID NO: 7 - protein, 8 aa, synthetic construct
GYTFTDYY

SEQ ID NO: 8 - protein, 8 aa, synthetic construct
INPNSGDT

SEQ ID NO: 9 - protein, 11 aa, synthetic construct
ARNSGSYSFGY

SEQ ID NO: 10 - protein, 6 aa, synthetic construct
QGISSW

SEQ ID NO: 11 - skipped

SEQ ID NO: 12 - protein, 9 aa, synthetic construct
QQAYSFPFT

SEQ ID NO: 13 - protein, 7 aa, synthetic construct
GYTFTDY

SEQ ID NO: 14 - protein, 6 aa, synthetic construct
NPNSGD

SEQ ID NO: 15 - protein, 9 aa, synthetic construct
NSGSYSFGY

SEQ ID NO: 16 - protein, 4 aa, synthetic construct
DYYM

SEQ ID NO: 17 - protein, 17 aa, synthetic construct
WINPNEGDTNYAQKFEG

SEQ ID NO: 18 - protein, 9 aa, synthetic construct
NTGAYSFGY

SEQ ID NO: 19 - protein, 7 aa, synthetic construct
GASDLQS

SEQ ID NO: 20 - protein, 9 aa, synthetic construct
QQAYGFPFT

SEQ ID NO: 21 - protein, 8 aa, synthetic construct
INPNEGDT

SEQ ID NO: 22 - protein, 11 aa, synthetic construct
ARNTGAYSFGY

SEQ ID NO: 23 - protein, 6 aa, synthetic construct
NPNEGD

SEQ ID NO: 24 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSS

SEQ ID NO: 25 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYSFPFTFGPGTKVDIE

SEQ ID NO: 26 - protein, 118 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGQGTLVTVSS

SEQ ID NO: 27 - protein, 107 aa, synthetic construct
DIQMTQSPSSVSASVGDRVTITCRASQGISSWLAWYQQKPGKAPKLLIYGASDLQSGVPSRFSGSGSGTDFTLTISSLQPEDFATYYCQQAYGFPFTFGPGTKVDIK

SEQ ID NO: 28 - protein, 447 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 214 aa, synthetic construct
DIQMTQSPSSVSASVGDRVTITCRASQGISSWLAWYQQKPGKAPKLLIYGASDLQSGVPSRFSGSGSGTDFTLTISSLQPEDFATYYCQQAYGFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 118 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGCGTLVTVSS

SEQ ID NO: 31 - protein, 107 aa, synthetic construct
DIQMTQSPSSVSASVGDRVTITCRASQGISSWLAWYQQKPGKCPKLLIYGASDLQSGVPSRFSGSGSGTDFTLTISSLQPEDFATYYCQQAYGFPFTFGPGTKVDIK

SEQ ID NO: 32 - protein, 447 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGCGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 214 aa, synthetic construct
DIQMTQSPSSVSASVGDRVTITCRASQGISSWLAWYQQKPGKCPKLLIYGASDLQSGVPSRFSGSGSGTDFTLTISSLQPEDFATYYCQQAYGFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 34 - protein, 248 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGCGTLVTVSSGGGGSGGGGSGGGGSGGGGSDIQMTQSPSSVSASVGDRVTITCRASQGISSWLAWYQQKPGKCPKLLIYGASDLQSGVPSRFSGSGSGTDFTLTISSLQPEDFATYYCQQAYGFPFTFGPGTKVDIKEPK

SEQ ID NO: 35 - protein, 476 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGCGTLVTVSSGGGGSGGGGSGGGGSGGGGSDIQMTQSPSSVSASVGDRVTITCRASQGISSWLAWYQQKPGKCPKLLIYGASDLQSGVPSRFSGSGSGTDFTLTISSLQPEDFATYYCQQAYGFPFTFGPGTKVDIKEPKRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 477 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGCGTLVTVSSGGGGSGGGGSGGGGSGGGGSDIQMTQSPSSVSASVGDRVTITCRASQGISSWLAWYQQKPGKCPKLLIYGASDLQSGVPSRFSGSGSGTDFTLTISSLQPEDFATYYCQQAYGFPFTFGPGTKVDIKEPKRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 6 aa, synthetic construct
SSNWWS

SEQ ID NO: 38 - protein, 16 aa, synthetic construct
EIYHDGSTDYFPSLKS

SEQ ID NO: 39 - protein, 7 aa, synthetic construct
DNWGFDY

SEQ ID NO: 40 - protein, 11 aa, synthetic construct
RASQSVSSSYL

SEQ ID NO: 41 - protein, 7 aa, synthetic construct
GASSRAT

SEQ ID NO: 42 - protein, 9 aa, synthetic construct
QQYGSSHRT

SEQ ID NO: 43 - protein, 9 aa, synthetic construct
GGSISSSNW

SEQ ID NO: 44 - protein, 7 aa, synthetic construct
IYHDGST

SEQ ID NO: 45 - protein, 9 aa, synthetic construct
ARDNWGFDY

SEQ ID NO: 46 - protein, 7 aa, synthetic construct
QSVSSSY

SEQ ID NO: 47 - skipped

SEQ ID NO: 48 - protein, 8 aa, synthetic construct
GGSISSSN

SEQ ID NO: 49 - protein, 5 aa, synthetic construct
YHDGS

SEQ ID NO: 50 - protein, 5 aa, synthetic construct
NWGFD

SEQ ID NO: 51 - protein, 8 aa, synthetic construct
SQSVSSSY

SEQ ID NO: 52 - protein, 6 aa, synthetic construct
YGSSHR

SEQ ID NO: 53 - protein, 5 aa, synthetic construct
SYGMN

SEQ ID NO: 54 - protein, 17 aa, synthetic construct
WINTNTGNPTYAQGFTG

SEQ ID NO: 55 - protein, 11 aa, synthetic construct
GYNWNDGDFDY

SEQ ID NO: 56 - protein, 11 aa, synthetic construct
RASQGIRNDLG

SEQ ID NO: 57 - protein, 7 aa, synthetic construct
AASSLQS

SEQ ID NO: 58 - protein, 9 aa, synthetic construct
LQNYNYPLT

SEQ ID NO: 59 - protein, 8 aa, synthetic construct
GYTFTSYG

SEQ ID NO: 60 - protein, 8 aa, synthetic construct
INTNTGNP

SEQ ID NO: 61 - protein, 13 aa, synthetic construct
ARGYNWNDGDFDY

SEQ ID NO: 62 - protein, 6 aa, synthetic construct
QGIRND

SEQ ID NO: 63 - skipped

SEQ ID NO: 64 - protein, 7 aa, synthetic construct
GYTFTSY

SEQ ID NO: 65 - protein, 6 aa, synthetic construct
NTNTGN

SEQ ID NO: 66 - protein, 9 aa, synthetic construct
YNWNDGDFD

SEQ ID NO: 67 - protein, 7 aa, synthetic construct
SQGIRND

SEQ ID NO: 68 - protein, 6 aa, synthetic construct
NYNYPL

SEQ ID NO: 69 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 70 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 71 - protein, 450 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 72 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 73 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 116 aa, synthetic construct
QVQLQESGPGLVKPSGTLSLTCAVSGGSISSSNWWSWVRQPPGKGLEWIGEIYHDGSTDYFPSLKSRVTISVDKSKNQFSLKLSSVTAADTAVYYCARDNWGFDYWGQGTLVTVSS

SEQ ID NO: 75 - protein, 108 aa, synthetic construct
DIVLTQSPGTLSLSPGERATLSCRASQSVSSSYLAWYQQKPGQAPRLLISGASSRATGIPDRFSGSGSGTDFTLTISRLEPEDFAVYYCQQYGSSHRTFGQGTKVEIK

SEQ ID NO: 76 - protein, 445 aa, synthetic construct
QVQLQESGPGLVKPSGTLSLTCAVSGGSISSSNWWSWVRQPPGKGLEWIGEIYHDGSTDYFPSLKSRVTISVDKSKNQFSLKLSSVTAADTAVYYCARDNWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 77 - protein, 446 aa, synthetic construct
QVQLQESGPGLVKPSGTLSLTCAVSGGSISSSNWWSWVRQPPGKGLEWIGEIYHDGSTDYFPSLKSRVTISVDKSKNQFSLKLSSVTAADTAVYYCARDNWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 78 - protein, 215 aa, synthetic construct
DIVLTQSPGTLSLSPGERATLSCRASQSVSSSYLAWYQQKPGQAPRLLISGASSRATGIPDRFSGSGSGTDFTLTISRLEPEDFAVYYCQQYGSSHRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 120 aa, synthetic construct
QVQLVQSGSELKKPGASVKLSCKASGYTFTSYGMNWVRQAPGQGLEWMGWINTNTGNPTYAQGFTGRFVFSLDTSVSTAYLQISSLKAEDTAVYYCARGYNWNDGDFDYWGQGTLVTVSS

SEQ ID NO: 80 - protein, 107 aa, synthetic construct
AIQMTQSPSSLSPSVGDRVTITCRASQGIRNDLGWYQQKPGKAPKLLIFAASSLQSGVPSRFSGSGSGTDFTLTISNLQPEDFATYYCLQNYNYPLTFGGGTKVEIK

SEQ ID NO: 81 - protein, 449 aa, synthetic construct
QVQLVQSGSELKKPGASVKLSCKASGYTFTSYGMNWVRQAPGQGLEWMGWINTNTGNPTYAQGFTGRFVFSLDTSVSTAYLQISSLKAEDTAVYYCARGYNWNDGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 82 - protein, 450 aa, synthetic construct
QVQLVQSGSELKKPGASVKLSCKASGYTFTSYGMNWVRQAPGQGLEWMGWINTNTGNPTYAQGFTGRFVFSLDTSVSTAYLQISSLKAEDTAVYYCARGYNWNDGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 83 - protein, 214 aa, synthetic construct
AIQMTQSPSSLSPSVGDRVTITCRASQGIRNDLGWYQQKPGKAPKLLIFAASSLQSGVPSRFSGSGSGTDFTLTISNLQPEDFATYYCLQNYNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 247 aa, synthetic construct
MEYHPDLENLDEDGYTQLHFDSQSNTRIAVVSEKGSCAASPPWRLIAVILGILCLVILVIAVVLGTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 85 - protein, 201 aa, synthetic construct
MEYHPDLENLDEDGYTQLHFDSQSNTRIAVVSEKGSCAASPPWRLIAVILGILCLVILVIAVVLGTMGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 86 - protein, 323 aa, synthetic construct
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 87 - protein, 447 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 448 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 214 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYSFPFTFGPGTKVDIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 448 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 448 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGCGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 12 aa, synthetic construct
GGGSGGGSGGGS

SEQ ID NO: 93 - protein, 5 aa, synthetic construct
GGGGS

SEQ ID NO: 94 - protein, 15 aa, synthetic construct
GGGGSGGGGSGGGGS

SEQ ID NO: 95 - protein, 20 aa, synthetic construct
GGGGSGGGGSGGGGSGGGGS

SEQ ID NO: 96 - protein, 5 aa, synthetic construct
GGGGS

SEQ ID NO: 97 - protein, 14 aa, synthetic construct
EPKRSDKTHTCPPC

SEQ ID NO: 98 - protein, 9 aa, synthetic construct
SATHTCPPC

SEQ ID NO: 99 - protein, 447 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 100 - protein, 448 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 101 - protein, 447 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGCGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 102 - protein, 448 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGCGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 103 - protein, 476 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGCGTLVTVSSGGGGSGGGGSGGGGSGGGGSDIQMTQSPSSVSASVGDRVTITCRASQGISSWLAWYQQKPGKCPKLLIYGASDLQSGVPSRFSGSGSGTDFTLTISSLQPEDFATYYCQQAYGFPFTFGPGTKVDIKEPKRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 104 - protein, 477 aa, synthetic construct
QVQLVQSGAEVKKPGASVKVSCKASGYTFTDYYMHWVRQAPGQGLEWMGWINPNEGDTNYAQKFEGRITMTRDTSISTAYMELSRLRSDDTAVYYCARNTGAYSFGYWGCGTLVTVSSGGGGSGGGGSGGGGSGGGGSDIQMTQSPSSVSASVGDRVTITCRASQGISSWLAWYQQKPGKCPKLLIYGASDLQSGVPSRFSGSGSGTDFTLTISSLQPEDFATYYCQQAYGFPFTFGPGTKVDIKEPKRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 105 - protein, 450 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 445 aa, synthetic construct
QVQLQESGPGLVKPSGTLSLTCAVSGGSISSSNWWSWVRQPPGKGLEWIGEIYHDGSTDYFPSLKSRVTISVDKSKNQFSLKLSSVTAADTAVYYCARDNWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - protein, 446 aa, synthetic construct
QVQLQESGPGLVKPSGTLSLTCAVSGGSISSSNWWSWVRQPPGKGLEWIGEIYHDGSTDYFPSLKSRVTISVDKSKNQFSLKLSSVTAADTAVYYCARDNWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 449 aa, synthetic construct
QVQLVQSGSELKKPGASVKLSCKASGYTFTSYGMNWVRQAPGQGLEWMGWINTNTGNPTYAQGFTGRFVFSLDTSVSTAYLQISSLKAEDTAVYYCARGYNWNDGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 450 aa, synthetic construct
QVQLVQSGSELKKPGASVKLSCKASGYTFTSYGMNWVRQAPGQGLEWMGWINTNTGNPTYAQGFTGRFVFSLDTSVSTAYLQISSLKAEDTAVYYCARGYNWNDGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 447 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 112 - protein, 448 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2026/0146093 A1
(43) Pub. Date: May 28, 2026

(54) MULTISPECIFIC BINDING PROTEINS THAT BIND DECTIN-1 AND TROP-2 AND METHODS OF USE THEREOF

(71) Applicant: Dren Bio Management, Inc., San Carlos, CA (US)

(72) Inventors: Nenad TOMASEVIC, Hillsborough, CA (US); Andrew P. AH YOUNG-CHAPON, South San Francisco, CA (US); Panagiotis FOTAKIS, Foster City, CA (US); Xiaodi DENG, Foster City, CA (US); Paula SO, Cupertino, CA (US)

(21) Appl. No.: 19/461,192

(22) Filed: Jan. 27, 2026

Related U.S. Application Data
(63) Continuation of application No. PCT/US2024/040192, filed on Jul. 30, 2024.
(60) Provisional application No. 63/530,018, filed on Jul. 31, 2023.

(51) Int. Cl. C07K 16/28; A61K 39/00; A61P 35/00; C07K 16/30

(52) U.S. Cl. CPC C07K 16/2851; A61P 35/00; C07K 16/30; A61K 2039/505; C07K 2317/31; C07K 2317/92

(57) ABSTRACT

The present disclosure relates to bispecific binding molecules comprising a first arm comprising a first antigen binding domain that binds to human Dectin-1 and a second arm comprising a second antigen binding domain that binds to human Trop-2, as well as methods, polynucleotides, vectors, host cells, compositions, and uses related thereto.

### CROSS-REFERENCE TO RELATED APPLICATIONS

This application is a continuation of International Application No. PCT/US2024/040192, filed Jul. 30, 2024, which claims priority to U.S. Provisional Application No. 63/530,018, filed Jul. 31, 2023, the disclosures of each of which are incorporated herein by reference in their entirety.

### REFERENCE TO AN ELECTRONIC SEQUENCE LISTING

The content of the electronic sequence listing (186542001001 seqlist.xml: Size: 111,873 bytes; and Date of Creation: Jan. 20, 2026) is incorporated herein by reference in its entirety.

### FIELD

The present disclosure relates to bispecific binding molecules comprising a first arm comprising a first antigen binding domain that binds to human Dectin-1 and a second arm comprising a second antigen binding domain that binds to human Trop-2, as well as methods, polynucleotides, vectors, host cells, compositions, and uses related thereto.

### BACKGROUND

Phagocytosis is a major mechanism used to remove pathogens and cell debris. Professional phagocytes, such as monocytes, macrophages, dendritic cells, and granulocytes, specifically recognize and engulf host or foreign agents that are aberrant or cause disease. The engulfed material is destroyed through the endo-lysosomal pathway in the phagocytes. Moreover, dendritic cells and macrophages can present antigens to the cells of the adaptive immune system to further promote the elimination of the disease-causing agents. Dectin-1 is a C-type lectin receptor that recognizes beta-glucans and promotes anti-fungal phagocytic activities. It is expressed on phagocytes and has been clearly shown to be sufficient for activating phagocytosis. Dectin-1 can be exploited for antibody-targeted phagocytosis and elimination of disease-causing agents.

Trop-2 (tumor-associated calcium signal transducer 2, also known as epithelial glycoprotein-1) is a transmembrane glycoprotein known to be overexpressed in many cancers (see, e.g., Shvartsur, A, and Bonavida, B. (2015) Genes Cancer 6 (3-4): 84-105). Overexpression of Trop-2 has been found to drive cancer growth by several pathways including activation of ERK/MAPK. In many cancers, Trop-2 overexpression has been linked to decreased survival, increased tumor aggressiveness, and metastasis. Cancers in which Trop-2 overexpression has been observed include breast, cervical, colorectal, endometrioid endometrial, esophageal, gastric, glioma, cholangiocarcinoma, chronic lymphocytic lymphoma (CLL), extranodal NK/T-cell lymphoma, non-Hodgkin's lymphoma, ovarian, prostate, pancreatic, thyroid, bladder, and uterine. Antibodies and antibody-drug conjugates targeting Trop-2 are under clinical testing, including sacituzumab govitecan, datopotamab deruxtecan, FDA018-ADC, JS108, and SKB264 (Shastry, M, et al. (2022) Breast 66:169-177).

Therefore, there remains a need for enhanced therapies that promote immune infiltration and elimination of Trop-2-expressing cancers.

All references cited herein, including patent applications, patent publications, and scientific literature, are herein incorporated by reference in their entirety, as if each individual reference were specifically and individually indicated to be incorporated by reference.

### BRIEF SUMMARY

The present disclosure relates to multispecific (e.g., bispecific) binding molecules comprising a first arm comprising a first antigen binding domain that binds to human Dectin-1 and a second arm comprising a second antigen binding domain that binds to human Trop-2, as well as methods, polynucleotides, vectors, host cells, compositions, and uses related thereto. The multispecific (e.g., bispecific) binding molecules allow the phagocyte to engage the target cell that expresses Trop-2 using the Trop-2 binding arm and form a synapse between it and promote clustering of Dectin-1 on the phagocyte using the Dectin-1 binding arm. This stimulates phagocytosis of the target cell and at the same time cytokine secretion via the Dectin-1/Syk/NfkB pathway by the phagocyte. Moreover, antigens from the engulfed material are presented on the surface of dendritic cells/macrophages to boost an adaptive immune response against the disease-causing agent that expresses Trop-2. Overall, it is thought that the multispecific (e.g., bispecific) binding molecules of the present disclosure promote immune stimulation, targeted phagocytosis, and neo-antigen presentation/activation of the adaptive immune system to eliminate disease-causing cells that express Trop-2.

As such, the present disclosure describes, inter alia, the generation and functional characterization of anti-Dectin-1 x anti-Trop-2 bispecific antibodies. These were found to bind human Trop-2 and Dectin-1 with high affinity, as well as activate human Dectin-1 signaling. Various formats were tested and demonstrated to specifically bind both targets, induce phagocytosis and depletion of Trop-2-expressing target cells (such as cancer cell lines), and inhibit tumor growth in multiple preclinical mouse models, including models of both “hot” and “cold” tumor types with respect to immune cell infiltration and responsiveness to immunotherapy (e.g., single agent immunotherapy).

In some embodiments, provided herein is a bispecific binding molecule, comprising: a first arm comprising a first antigen binding domain that binds to human Dectin-1; and a second arm comprising a second antigen binding domain that binds to human Trop-2; wherein the second antigen binding domain comprises a second heavy chain variable (VH) domain and a second light chain variable (VL) domain.

In some embodiments according to any of the embodiments described herein, the second VH domain comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39, and the second VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the second VH domain comprises a CDR-H1 comprising the amino acid sequence of SEQ ID NO:48, a CDR-H2 comprising the amino acid sequence of SEQ ID NO:49, and a CDR-H3 comprising the amino acid sequence of SEQ ID NO:50, and the second VL domain comprises a CDR-L1 comprising the amino acid sequence of SEQ ID NO:51, a CDR-L2 comprising the amino acid sequence of GAS, and a CDR-L3 comprising the amino acid sequence of SEQ ID NO:52. In some embodiments, the second VH domain comprises a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO: 45, and the second VL domain comprises a CDR-L1 comprising the amino acid sequence of SEQ ID NO: 46, a CDR-L2 comprising the amino acid sequence of GAS, and a CDR-L3 comprising the amino acid sequence of SEQ ID NO:42. In some embodiments, the second VH domain comprises the amino acid sequence of SEQ ID NO: 74, and the second VL domain comprises the amino acid sequence of SEQ ID NO: 75.

In some embodiments according to any of the embodiments described herein, the second VH domain comprises a CDR-H1 comprising the amino acid sequence of SEQ ID NO:53, a CDR-H2 comprising the amino acid sequence of SEQ ID NO:54, and a CDR-H3 comprising the amino acid sequence of SEQ ID NO:55, and the second VL domain comprises a CDR-L1 comprising the amino acid sequence of SEQ ID NO:56, a CDR-L2 comprising the amino acid sequence of SEQ ID NO:57, and a CDR-L3 comprising the amino acid sequence of SEQ ID NO:58. In some embodiments, the second VH domain comprises a CDR-H1 comprising the amino acid sequence of SEQ ID NO:64, a CDR-H2 comprising the amino acid sequence of SEQ ID NO:65, and a CDR-H3 comprising the amino acid sequence of SEQ ID NO:66, and the second VL domain comprises a CDR-L1 comprising the amino acid sequence of SEQ ID NO:67, a CDR-L2 comprising the amino acid sequence of AAS, and a CDR-L3 comprising the amino acid sequence of SEQ ID NO:68. In some embodiments, the second VH domain comprises a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO: 61, and the second VL domain comprises a CDR-L1 comprising the amino acid sequence of SEQ ID NO: 62, a CDR-L2 comprising the amino acid sequence of AAS, and a CDR-L3 comprising the amino acid sequence of SEQ ID NO:68. In some embodiments, the second VH domain comprises the amino acid sequence of SEQ ID NO: 79, and the second VL domain comprises the amino acid sequence of SEQ ID NO: 80.

In some embodiments according to any of the embodiments described herein, the second arm comprises an antibody heavy chain polypeptide that comprises the second VH domain and an antibody light chain polypeptide that comprises the second VL domain. In some embodiments, the antibody heavy chain polypeptide of the second arm comprises an Fc region. In some embodiments, the Fc region of the second arm is a human IgG Fc region. In some embodiments, the Fc region of the second arm is a human IgG1 Fc region. In some embodiments, the Fc region is a human IgG1 Fc region comprising S239D and 1332E substitutions, according to EU numbering. In some embodiments, the Fc region is a human IgG1 Fc region comprising S239D. A330L, and 1332E substitutions, according to EU numbering. In some embodiments, the Fc region is a human IgG1 Fc region comprising G236A, S239D, A330L, and 1332E substitutions, according to EU numbering. In some embodiments, the Fc region of the second arm is a human IgG4 Fc region. In some embodiments, the Fc region is a human IgG4 Fc region comprising an S228P substitution, according to EU numbering. In some embodiments, the first arm comprises an Fc region; wherein the Fc region of the first arm comprises one or more knob-forming mutations, and the Fc region of the second arm comprises one or more cognate hole-forming mutations. In some embodiments, the Fc region of the first arm comprises a T366W substitution, and wherein the Fc region of the second arm comprises T366S, L368A, and Y407V substitutions, according to EU numbering. In some embodiments, the first arm comprises an Fc region; wherein the Fc region of the second arm comprises one or more knob-forming mutations, and the Fc region of the first arm comprises one or more cognate hole-forming mutations. In some embodiments, the Fc region of the second arm comprises a T366W substitution, and wherein the Fc region of the first arm comprises T366S, L368A, and Y407V substitutions, according to EU numbering.

In some embodiments according to any of the embodiments described herein, the second arm comprises an antibody heavy chain polypeptide that comprises the amino acid sequence of SEQ ID NO:76 or 77 and an antibody light chain polypeptide that comprises the amino acid sequence of SEQ ID NO:78. In some embodiments, the second arm comprises an antibody heavy chain polypeptide that comprises the amino acid sequence of SEQ ID NO:81 or 82 and an antibody light chain polypeptide that comprises the amino acid sequence of SEQ ID NO:83.

In some embodiments, provided herein is a bispecific binding molecule, comprising: a first arm comprising a first antigen binding domain that binds to human Dectin-1; and a second arm comprising a second antigen binding domain that binds to human Trop-2; wherein the second arm comprises an antibody heavy chain polypeptide that comprises the amino acid sequence of SEQ ID NO:71 or 72 and an antibody light chain polypeptide that comprises the amino acid sequence of SEQ ID NO:73.

In some embodiments according to any of the embodiments described herein, the first antigen binding domain comprises a first heavy chain variable (VH) domain and a first light chain variable (VL) domain. In some embodiments, the first VH domain comprises a CDR-H1 comprising the amino acid sequence of SEQ ID NO: 1, a CDR-H2 comprising the amino acid sequence of SEQ ID NO:2, and a CDR-H3 comprising the amino acid sequence of SEQ ID NO:3, and the second VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6. In some embodiments, the first VH domain comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO:15, and the second VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6. In some embodiments, the first VH domain comprises a CDR-H1 comprising the amino acid sequence of SEQ ID NO:7, a CDR-H2 comprising the amino acid sequence of SEQ ID NO:8, and a CDR-H3 comprising the amino acid sequence of SEQ ID NO:9, and the second VL domain comprises a CDR-L1 comprising the amino acid sequence of SEQ ID NO: 10, a CDR-L2 comprising the amino acid sequence of GAS, and a CDR-L3 comprising the amino acid sequence of SEQ ID NO: 12. In some embodiments, the first VH domain comprises the amino acid sequence of SEQ ID NO:24, and the first VL domain comprises the amino acid sequence of SEQ ID NO:25.

In some embodiments according to any of the embodiments described herein, the first VH domain comprises a CDR-H1 comprising the amino acid sequence of SEQ ID NO: 16, a CDR-H2 comprising the amino acid sequence of SEQ ID NO: 17, and a CDR-H3 comprising the amino acid sequence of SEQ ID NO: 18, and the second VL domain comprises a CDR-L1 comprising the amino acid sequence of SEQ ID NO:4, a CDR-L2 comprising the amino acid sequence of SEQ ID NO: 19, and a CDR-L3 comprising the amino acid sequence of SEQ ID NO:20. In some embodiments, the first VH domain comprises a CDR-H1 comprising the amino acid sequence of SEQ ID NO: 13, a CDR-H2 comprising the amino acid sequence of SEQ ID NO:23, and a CDR-H3 comprising the amino acid sequence of SEQ ID NO: 18, and the second VL domain comprises a CDR-L1 comprising the amino acid sequence of SEQ ID NO:4, a CDR-L2 comprising the amino acid sequence of SEQ ID NO: 19, and a CDR-L3 comprising the amino acid sequence of SEQ ID NO:20. In some embodiments, the first VH domain comprises a CDR-H1 comprising the amino acid sequence of SEQ ID NO:7, a CDR-H2 comprising the amino acid sequence of SEQ ID NO:21, and a CDR-H3 comprising the amino acid sequence of SEQ ID NO:22, and the second VL domain comprises a CDR-L1 comprising the amino acid sequence of SEQ ID NO: 10, a CDR-L2 comprising the amino acid sequence of GAS, and a CDR-L3 comprising the amino acid sequence of SEQ ID NO:20. In some embodiments, the first VH domain comprises the amino acid sequence of SEQ ID NO:26, and the first VL domain comprises the amino acid sequence of SEQ ID NO:27. In some embodiments, the first VH domain comprises the amino acid sequence of SEQ ID NO:30, and the first VL domain comprises the amino acid sequence of SEQ ID NO: 31.

In some embodiments according to any of the embodiments described herein, the first arm comprises an antibody heavy chain polypeptide that comprises the first VH domain and an antibody light chain polypeptide that comprises the first VL domain. In some embodiments, the antibody heavy chain polypeptide of the first arm comprises an Fc region. In some embodiments, the first arm comprises a single chain variable fragment (scFv) that comprises the first VH domain and the first VL domain. In some embodiments, the scFv comprises a linker between the first VH domain and the first VL domain. In some embodiments, the scFv comprises the amino acid sequence of SEQ ID NO:34. In some embodiments, the first arm further comprises an Fc region linked to the scFv. In some embodiments, the Fc region of the first arm is a human IgG Fc region. In some embodiments, the Fc region of the first arm is a human IgG1 Fc region. In some embodiments, the Fc region is a human IgG1 Fc region comprising S239D and 1332E substitutions, according to EU numbering. In some embodiments, the Fc region is a human IgG1 Fc region comprising S239D, A330L, and 1332E substitutions, according to EU numbering. In some embodiments, the Fc region is a human IgG1 Fc region comprising G236A, S239D, A330L, and 1332E substitutions, according to EU numbering. In some embodiments, the Fc region of the first arm is a human IgG4 Fc region. In some embodiments, the Fc region is a human IgG4 Fc region comprising an S228P substitution, according to EU numbering. In some embodiments, the second arm comprises an Fc region; wherein the Fc region of the first arm comprises one or more knob-forming mutations, and the Fc region of the second arm comprises one or more cognate hole-forming mutations. In some embodiments, the Fc region of the first arm comprises a T366W substitution, and wherein the Fc region of the second arm comprises T366S, L368A, and Y407V substitutions, according to EU numbering. In some embodiments, the second arm comprises Fc region; wherein the Fc region of the second arm comprises one or more knob-forming mutations, and the Fc region of the first arm comprises one or more cognate hole-forming mutations. In some embodiments, the Fc region of the second arm comprises a T366W substitution, and wherein the Fc region of the first arm comprises T366S, L368A, and Y407V substitutions, according to EU numbering.

In some embodiments according to any of the embodiments described herein, the first arm comprises an antibody heavy chain polypeptide that comprises the amino acid sequence of SEQ ID NO:87 or 88 and an antibody light chain polypeptide that comprises the amino acid sequence of SEQ ID NO:89. In some embodiments, the first arm comprises an antibody heavy chain polypeptide that comprises the amino acid sequence of SEQ ID NO: 111 or 112 and an antibody light chain polypeptide that comprises the amino acid sequence of SEQ ID NO:89. In some embodiments, the first arm comprises an antibody heavy chain polypeptide that comprises the amino acid sequence of SEQ ID NO:28 or 90 and an antibody light chain polypeptide that comprises the amino acid sequence of SEQ ID NO:29. In some embodiments, the first arm comprises an antibody heavy chain polypeptide that comprises the amino acid sequence of SEQ ID NO:99 or 100 and an antibody light chain polypeptide that comprises the amino acid sequence of SEQ ID NO:29. In some embodiments, the first arm comprises an antibody heavy chain polypeptide that comprises the amino acid sequence of SEQ ID NO:32 or 91 and an antibody light chain polypeptide that comprises the amino acid sequence of SEQ ID NO:33. In some embodiments, the first arm comprises the amino acid sequence of SEQ ID NO:35 or 36. In some embodiments, the first arm comprises an antibody heavy chain polypeptide that comprises the amino acid sequence of SEQ ID NO: 101 or 102 and an antibody light chain polypeptide that comprises the amino acid sequence of SEQ ID NO:33. In some embodiments, the first arm comprises the amino acid sequence of SEQ ID NO:35 or 36. In some embodiments, the first arm comprises the amino acid sequence of SEQ ID NO: 103 or 104.

In some embodiments according to any of the embodiments described herein, at least one or two of the first and second arms is/are non-fucosylated or comprise(s) reduced fucosylation. In some embodiments, human Dectin-1 comprises the amino acid sequence of SEQ ID NO:84 or 85. In some embodiments, human Trop-2 comprises the amino acid sequence of SEQ ID NO:86.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:87 or 88, the first antibody light chain polypeptide comprises the amino acid sequence of SEQ ID NO:89, the second antibody heavy chain polypeptide comprises the amino acid sequence of SEQ ID NO:71 or 72, and the second antibody light chain polypeptide comprises the amino acid sequence of SEQ ID NO:73.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO: 111 or 112, the first antibody light chain polypeptide comprises the amino acid sequence of SEQ ID NO: 89, the second antibody heavy chain polypeptide comprises the amino acid sequence of SEQ ID NO: 105 or 106, and the second antibody light chain polypeptide comprises the amino acid sequence of SEQ ID NO: 73.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:87 or 88, the first antibody light chain polypeptide comprises the amino acid sequence of SEQ ID NO:89, the second antibody heavy chain polypeptide comprises the amino acid sequence of SEQ ID NO:76 or 77, and the second antibody light chain polypeptide comprises the amino acid sequence of SEQ ID NO:78.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO: 111 or 112, the first antibody light chain polypeptide comprises the amino acid sequence of SEQ ID NO: 89, the second antibody heavy chain polypeptide comprises the amino acid sequence of SEQ ID NO: 107 or 108, and the second antibody light chain polypeptide comprises the amino acid sequence of SEQ ID NO:78.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:87 or 88, the first antibody light chain polypeptide comprises the amino acid sequence of SEQ ID NO:89, the second antibody heavy chain polypeptide comprises the amino acid sequence of SEQ ID NO:81 or 82, and the second antibody light chain polypeptide comprises the amino acid sequence of SEQ ID NO:83.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO: 111 or 112, the first antibody light chain polypeptide comprises the amino acid sequence of SEQ ID NO: 89, the second antibody heavy chain polypeptide comprises the amino acid sequence of SEQ ID NO: 109 or 110, and the second antibody light chain polypeptide comprises the amino acid sequence of SEQ ID NO:83.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:28 or 90, the first antibody light chain polypeptide comprises the amino acid sequence of SEQ ID NO:29, the second antibody heavy chain polypeptide comprises the amino acid sequence of SEQ ID NO:71 or 72, and the second antibody light chain polypeptide comprises the amino acid sequence of SEQ ID NO: 73.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:99 or 100, the first antibody light chain polypeptide comprises the amino acid sequence of SEQ ID NO:29, the second antibody heavy chain polypeptide comprises the amino acid sequence of SEQ ID NO: 105 or 106, and the second antibody light chain polypeptide comprises the amino acid sequence of SEQ ID NO: 73.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:28 or 90, the first antibody light chain polypeptide comprises the amino acid sequence of SEQ ID NO:29, the second antibody heavy chain polypeptide comprises the amino acid sequence of SEQ ID NO:76 or 77, and the second antibody light chain polypeptide comprises the amino acid sequence of SEQ ID NO:78.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:99 or 100, the first antibody light chain polypeptide comprises the amino acid sequence of SEQ ID NO:29, the second antibody heavy chain polypeptide comprises the amino acid sequence of SEQ ID NO: 107 or 108, and the second antibody light chain polypeptide comprises the amino acid sequence of SEQ ID NO: 78.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:28 or 90, the first antibody light chain polypeptide comprises the amino acid sequence of SEQ ID NO:29, the second antibody heavy chain polypeptide comprises the amino acid sequence of SEQ ID NO:81 or 82, and the second antibody light chain polypeptide comprises the amino acid sequence of SEQ ID NO:83.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:99 or 100, the first antibody light chain polypeptide comprises the amino acid sequence of SEQ ID NO:29, the second antibody heavy chain polypeptide comprises the amino acid sequence of SEQ ID NO: 109 or 110, and the second antibody light chain polypeptide comprises the amino acid sequence of SEQ ID NO: 83.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:32 or 91, the first antibody light chain polypeptide comprises the amino acid sequence of SEQ ID NO:33, the second antibody heavy chain polypeptide comprises the amino acid sequence of SEQ ID NO:71 or 72, and the second antibody light chain polypeptide comprises the amino acid sequence of SEQ ID NO: 73.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO: 101 or 102, the first antibody light chain polypeptide comprises the amino acid sequence of SEQ ID NO: 33, the second antibody heavy chain polypeptide comprises the amino acid sequence of SEQ ID NO: 105 or 106, and the second antibody light chain polypeptide comprises the amino acid sequence of SEQ ID NO: 73.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:32 or 91, the first antibody light chain polypeptide comprises the amino acid sequence of SEQ ID NO:33, the second antibody heavy chain polypeptide comprises the amino acid sequence of SEQ ID NO: 76 or 77, and the second antibody light chain polypeptide comprises the amino acid sequence of SEQ ID NO:78.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO: 101 or 102, the first antibody light chain polypeptide comprises the amino acid sequence of SEQ ID NO: 33, the second antibody heavy chain polypeptide comprises the amino acid sequence of SEQ ID NO: 107 or 108, and the second antibody light chain polypeptide comprises the amino acid sequence of SEQ ID NO:78.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO:32 or 91, the first antibody light chain polypeptide comprises the amino acid sequence of SEQ ID NO:33, the second antibody heavy chain polypeptide comprises the amino acid sequence of SEQ ID NO:81 or 82, and the second antibody light chain polypeptide comprises the amino acid sequence of SEQ ID NO:83.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a first antibody heavy chain polypeptide and a first antibody light chain polypeptide, and the second arm comprises a second antibody heavy chain polypeptide and a second antibody light chain polypeptide; and wherein the first antibody heavy chain polypeptide comprises the amino acid sequence of SEQ ID NO: 101 or 102, the first antibody light chain polypeptide comprises the amino acid sequence of SEQ ID NO: 33, the second antibody heavy chain polypeptide comprises the amino acid sequence of SEQ ID NO: 109 or 110, and the second antibody light chain polypeptide comprises the amino acid sequence of SEQ ID NO:83.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a single chain variable fragment (scFv) linked to an Fc region, and the second arm comprises an antibody heavy chain polypeptide and an antibody light chain polypeptide; and wherein the first arm comprises the amino acid sequence of SEQ ID NO:35 or 36, the antibody heavy chain polypeptide comprises the amino acid sequence of SEQ ID NO:71 or 72, and the antibody light chain polypeptide comprises the amino acid sequence of SEQ ID NO: 73.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a single chain variable fragment (scFv) linked to an Fc region, and the second arm comprises an antibody heavy chain polypeptide and an antibody light chain polypeptide; and wherein the first arm comprises the amino acid sequence of SEQ ID NO: 103 or 104, the antibody heavy chain polypeptide comprises the amino acid sequence of SEQ ID NO: 105 or 106, and the antibody light chain polypeptide comprises the amino acid sequence of SEQ ID NO:73.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a single chain variable fragment (scFv) linked to an Fc region, and the second arm comprises an antibody heavy chain polypeptide and an antibody light chain polypeptide; and wherein the first arm comprises the amino acid sequence of SEQ ID NO:35 or 36, the antibody heavy chain polypeptide comprises the amino acid sequence of SEQ ID NO:76 or 77, and the antibody light chain polypeptide comprises the amino acid sequence of SEQ ID NO:78.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a single chain variable fragment (scFv) linked to an Fc region, and the second arm comprises an antibody heavy chain polypeptide and an antibody light chain polypeptide; and wherein the first arm comprises the amino acid sequence of SEQ ID NO: 103 or 104, the antibody heavy chain polypeptide comprises the amino acid sequence of SEQ ID NO: 107 or 108, and the antibody light chain polypeptide comprises the amino acid sequence of SEQ ID NO:78.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a single chain variable fragment (scFv) linked to an Fc region, and the second arm comprises an antibody heavy chain polypeptide and an antibody light chain polypeptide; and wherein the first arm comprises the amino acid sequence of SEQ ID NO:35 or 36, the antibody heavy chain polypeptide comprises the amino acid sequence of SEQ ID NO:81 or 82, and the antibody light chain polypeptide comprises the amino acid sequence of SEQ ID NO:83.

In some embodiments, provided herein is a bispecific binding molecule, comprising: a first arm comprising a first antigen binding domain that binds to human Dectin-1; and a second arm comprising a second antigen binding domain that binds to human Trop-2; wherein the first arm comprises a single chain variable fragment (scFv) linked to an Fc region, and the second arm comprises an antibody heavy chain polypeptide and an antibody light chain polypeptide; and wherein the first arm comprises the amino acid sequence of SEQ ID NO: 103 or 104, the antibody heavy chain polypeptide comprises the amino acid sequence of SEQ ID NO: 109 or 110, and the antibody light chain polypeptide comprises the amino acid sequence of SEQ ID NO:83.

Further provided herein is a polynucleotide encoding the bispecific binding molecule according to any one of the above embodiments. Further provided herein is a vector comprising the polynucleotide according to any one of the above embodiments, e.g., an expression vector. Further provided herein is a host cell (e.g., an isolated host cell) comprising the polynucleotide or vector according to any one of the above embodiments. In some embodiments, the cell is a yeast, insect, plant, or prokaryotic cell. In some embodiments, the host cell is a mammalian cell, e.g., a Chinese hamster ovary (CHO) cell. In some embodiments, the host cell comprises an alpha1,6-fucosyltransferase (Fut8) or alpha-1,3-mannosyl-glycoprotein 2-beta-N-acetylglucosaminyltranferase (MGAT1) knockout. In some embodiments, the host cell overexpresses β1,4-N-acetylglucosaminyltransferase III (GnT-III) and optionally further overexpresses Golgi μ-mannosidase II (ManII). Further provided herein is a method of producing a bispecific binding molecule, comprising culturing the host cell according to any one of the above embodiments under conditions suitable for production of the bispecific binding molecule. In some embodiments, the method further comprises recovering the bispecific binding molecule from the host cell. In some embodiments, prior to production of the bispecific binding molecule, the host cell is treated with kifunensine. Further provided herein is a bispecific binding molecule produced by the method according to any one of the above embodiments. Further provided herein is a pharmaceutical composition comprising the bispecific binding molecule according to any one of the above embodiments and a pharmaceutically acceptable carrier.

Further provided herein is a method of treating cancer, comprising administering an effective amount of the bispecific binding molecule or pharmaceutical composition according to any one of the above embodiments to an individual in need thereof. Further provided herein is the bispecific binding molecule or pharmaceutical composition according to any one of the above embodiments for use in a method of treating cancer, said method comprising administering an effective amount of the bispecific binding molecule or pharmaceutical composition to an individual in need thereof. Further provided herein is the use of the bispecific binding molecule or pharmaceutical composition according to any one of the above embodiments in the manufacture of a medicament for treating cancer in an individual. In some embodiments, the individual has or has been diagnosed with cancer. In some embodiments, the cancer is a solid cancer. In some embodiments, the cancer is breast cancer, prostate cancer, urothelial cancer, cervical cancer, bladder cancer, pancreatic cancer, thyroid cancer, head and neck cancer, esophageal cancer, endometrial cancer, gastric cancer, liver cancer, colorectal cancer, skin cancer (e.g., melanoma), lung cancer, ovarian cancer, or uterine cancer. In some embodiments, the cancer is cervical squamous cell carcinoma, bladder urothelial carcinoma, head and neck squamous cell carcinoma (HNSCC), esophageal squamous cell carcinoma, endocervical adenocarcinoma, lung squamous cell carcinoma, prostate adenocarcinoma, papillary thyroid cancer, mucinous carcinoma, endometrioid carcinoma, pancreatic adenocarcinoma, breast invasive lobular carcinoma, lung adenocarcinoma, uterine endometrioid carcinoma, serous ovarian cancer, breast invasive carcinoma (NOS), breast invasive ductal carcinoma, uterine serous carcinoma, metaplastic breast cancer, uterine mixed endometrial carcinoma, breast invasive mixed mucinous carcinoma, papillary renal cell carcinoma, esophageal adenocarcinoma, stomach adenocarcinoma, invasive breast carcinoma, intrahepatic cholangiocarcinoma, cholangiocarcinoma, tubular stomach adenocarcinoma, intestinal type stomach adenocarcinoma, mucinous stomach adenocarcinoma, papillary stomach adenocarcinoma, diffuse type stomach adenocarcinoma, teratoma, signet ring cell carcinoma of the stomach, mucinous adenocarcinoma of the colon/rectum, uterine carcinosarcoma, rectal adenocarcinoma, thymoma, or perihilar cholangiocarcinoma. In some embodiments, cells of the cancer express Trop-2. In some embodiments, the individual is a human. In some embodiments, the cancer is responsive to single-agent immunotherapy (e.g., single-agent treatment with immune checkpoint inhibitor), e.g., prior to administration of the bispecific binding molecule. In some embodiments, the cancer is not responsive to single-agent immunotherapy (e.g., single-agent treatment with immune checkpoint inhibitor), e.g., prior to administration of the bispecific binding molecule.

Further provided herein is a kit comprising the bispecific binding molecule or pharmaceutical composition according to any one of the above embodiments. In some embodiments, the kit further comprises instructions for using the bispecific binding molecule or pharmaceutical composition to treat cancer in an individual, e.g., according to any one of the methods disclosed herein.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIGS. 1A-1C show exemplary formats for anti-Dectin-1 x anti-Trop-2 bispecific antibodies, in accordance with some embodiments. FIG. 1A shows an embodiment in which both arms comprise Fabs (Fab/Fab). Engineered disulfides can also be used in this format to drive pairing of the correct heavy and light chain variable domains. For example, cysteines forming native disulfide bonds between heavy and light chains can be removed from one arm, and cysteine substitutions can be introduced to give rise to non-native disulfide bonds. The other arm can have native disulfide bonds, thereby distinguishing the variable domains for each arm by disulfide bonding position. FIG. 1B shows an embodiment that has an interchain disulfide bond engineered in the Dectin-1 binding arm between the VH and VL domains. whereas the Trop-2 binding arm has the native interchain disulfide between CH1 and CL domains (interchain disulfide bonds indicated by arrows). FIG. 1C shows an embodiment in which one arm (in this case, the anti-Dectin-1 arm) comprises a single chain variable fragment (scFv), and the other arm (in this case, the anti-Trop-2 arm) comprises a Fab (scFv/Fab). In all examples, the bispecific antibody further comprises an Fc region (in this examples, human IgG1).

FIGS. 2A-2D show binding assays in which cells expressing human Trop-2 were incubated with anti-Dectin-1 x anti-Trop-2 bispecific antibodies with human IgG1 Fc (as indicated) or isotype control, and binding was measured by flow cytometry using a fluorescently labeled anti-human secondary antibody. Shown are binding of the indicated antibodies to human epidermoid carcinoma A-431 cells (FIG. 2A), human gastric carcinoma NCI-N87 cells (FIG. 2B), human breast cancer SKBR3 cells (FIG. 2C), or CHO-K1 cells expressing human Trop-2 (FIG. 2D).

FIGS. 3A-3D show activation of the Dectin-1 pathway by anti-Dectin-1 x anti-Trop-2 bispecific antibodies with human IgG1 Fc (as indicated) or isotype control. Human cancer cell lines expressing hTrop-2 were treated with a serial dilution of the indicated bispecific antibody or isotype control in the presence of HEK-Blue hDectin-1a cells engineered to express Dectin-1 isoform A and genes involved in the Dectin-1/NF-κB/SEAP signaling pathway (thus expressing a secreted alkaline phosphatase (SEAP) in response to stimulation by Dectin-1 ligands). Shown are activation of Dectin-1 signaling in HEK-Bluc hDectin-1a cells (as measured by SEAP) in the presence of A-431 cells (FIG. 3A), NCI-N87 cells (FIG. 3B). SKBR3 cells (FIG. 3C), or CHO-K1 cells expressing human Trop-2 (FIG. 3D) upon incubation with the indicated antibody.

FIGS. 4A-4C show cell binding and Dectin-1 activation by anti-Dectin-1 x anti-Trop-2 bispecific antibodies with human IgG1 Fc (as indicated) or isotype control using the scFv format shown in FIG. 1C. FIG. 4A shows binding of the indicated anti-Dectin-1 x anti-Trop-2 bispecific antibodies to HEK cells expressing hDectin-1 as measured by flow cytometry. FIG. 4B shows binding of the indicated anti-Dectin-1 x anti-Trop-2 bispecific antibodies to A431 cells expressing hTrop-2 as measured by flow cytometry. FIG. 4C shows Dectin-1 activation by the indicated anti-Dectin-1 x anti-Trop-2 bispecific antibodies as measured by SEAP secretion; EC50 values are also shown.

FIGS. 5A-5C show cell binding and Dectin-1 activation by anti-Dectin-1 x anti-Trop-2 bispecific antibodies with human IgG1 Fc (as indicated) or isotype control using the Fab format shown in FIG. 1B with engineered disulfides in the anti-Dectin-1 arm. FIG. 5A shows binding of the indicated anti-Dectin-1 x anti-Trop-2 bispecific antibodies to HEK cells expressing hDectin-1 as measured by flow cytometry. FIG. 5B shows binding of the indicated anti-Dectin-1 x anti-Trop-2 bispecific antibodies to CHO-K1 cells expressing hTrop-2 as measured by flow cytometry. FIG. 5C shows Dectin-1 activation by the indicated anti-Dectin-1 x anti-Trop-2 bispecific antibodies as measured by SEAP secretion; EC50 values are also shown

FIGS. 6A-6C show induction of phagocytosis by anti-Dectin-1 x anti-Trop-2 bispecific antibodies with human IgG1 Fc (as indicated) or isotype control using the Fab format shown in FIG. 1A. Human macrophages and calcein AM-labeled human cancer cells were co-cultured (3:1 ratio) in the presence of the indicated anti-Dectin-1 x anti-Trop-2 bispecific antibody at 37° C. for 24 hours, then stained with PE-labeled anti-CD206 antibody to label macrophages. Phagocytosis was quantified as the percentage of FITC+ cancer cells engulfed by macrophages in the single gate. Antibodies were tested in serial dilution. FIG. 6A shows phagocytosis of A431 cells by human macrophages induced by the indicated anti-Dectin-1 x anti-Trop-2 bispecific antibodies or isotype control. FIG. 6B shows phagocytosis of NCI-N87 cells by human macrophages induced by the indicated anti-Dectin-1 x anti-Trop-2 bispecific antibodies or isotype control. FIG. 6C shows phagocytosis of SKBR3 cells by human macrophages induced by the indicated anti-Dectin-1 x anti-Trop-2 bispecific antibodies or isotype control.

FIGS. 7A-7D show cytokine secretion by PBMCs co-cultured with CHO-K1 cells expressing hTrop-2 in response to treatment with anti-Dectin-1 x anti-Trop-2 bispecific antibodies with human IgG1 Fc (as indicated) or isotype control using the Fab format shown in FIG. 1A. Dose-response of TNF-alpha secretion to treatment with anti-Dectin-1 x anti-Trop-2 bispecific antibodies with human IgG1 Fc (as indicated) is shown in FIG. 7A. Dose-response of IL-6 secretion to treatment with anti-Dectin-1 x anti-Trop-2 bispecific antibodies with human IgG1 Fc (as indicated) is shown in FIG. 7B. Dose-response of IFN-gamma secretion to treatment with anti-Dectin-1 x anti-Trop-2 bispecific antibodies with human IgG1 Fc (as indicated) is shown in FIG. 7C. Summary of all cytokines using PBMCs from both donors is shown in FIG. 7D.

FIGS. 8A & 8B show activity of anti-Dectin-1 x anti-Trop-2 bispecific antibodies (using anti-mouse Dectin-1 arm and mouse IgG2a Fc) in syngencic mouse tumor xenograft models. Eight-week-old female Balb/c mice or C57BL6 mice were inoculated subcutaneously with 2 million mouse colon cancer cells overexpressing human Trop2 (hTrop2-CT26) or 0.5 million mouse melanoma cancer cells overexpressing human Trop2 (hTrop2-B16F10), respectively. When tumors reached 80 mm³ the mice were randomized into 4 groups of 12 mice each and received six doses of anti-Dectin-1 x anti-Trop-2 bispecific antibody with anti-mDectin-1 Fab (“Dectin-1 surr”) and anti-Trop-2 clone A, B, or C along with mIgG2a Fc (or isotype control) intraperitoneally at 10 mg/kg twice every week. The tumor volume and body weight were measured 2-3 times every week. FIG. 8A shows median tumor volume growth in mice inoculated with hTrop-2-CT26 cells treated with anti-Dectin-1 x anti-Trop-2 bispecific antibody or mIgG2a isotype control. FIG. 8B shows median tumor volume growth in mice inoculated with hTrop-2-B16F10 cells treated with anti-Dectin-1 x anti-Trop-2 bispecific antibody or mIgG2a isotype control.

FIG. 9 shows activity of anti-Dectin-1 x anti-Trop-2 bispecific antibodies (using disulfide-engineered anti-hDectin-1 arm and mouse IgG2a Fc) against hTrop2-B16F10 tumor xenografts in syngeneic mouse model with mice expressing hDectin-1. Tumor volume over time is shown.

FIGS. 10A-10D show depletion of cancer cells from single cell suspensions derived from tissue biopsies using anti-Dectin-1 x anti-Trop-2 bispecific antibodies. FIGS. 10A-10C show % of cancer cells remaining (as % of live cells) after 24 h. incubation of single cell suspensions with anti-Dectin-1 x anti-Trop-2 bispecific antibodies (as indicated) from lung cancer (FIG. 10A), ovarian cancer (FIG. 10B), or uterine cancer (FIG. 10C) biopsies. FIG. 10D shows average expression (mRNA) of Trop-2 across indicated cancer types from TCGA PanCancer Atlas study data.

### DETAILED DESCRIPTION

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing”, and is inclusive or open-ended.

Any reference to “or” herein is intended to encompass “and/or” unless otherwise stated. As used herein, the term “about” with reference to a number refers to that number plus or minus 10% of that number. The term “about” with reference to a range refers to that range minus 10% of its lowest value and plus 10% of its greatest value.

In some embodiments, antibody and immunoglobulin are used interchangeably and herein are used in the broadest sense and encompass various antibody structures, including but not limited to monoclonal antibodies (e.g., full length or intact monoclonal antibodies), polyclonal antibodies, multispecific antibodies (e.g., bispecific antibodies), antibody fragments and single domain antibody (as described in greater detail herein), so long as they exhibit the desired antigen binding activity.

In some embodiments, antibodies (immunoglobulins) refer to a protein having a structure substantially similar to a native antibody structure, or a protein having heavy and light chain variable regions having structures substantially similar to native heavy and light chain variable region structures. Native antibodies refer to naturally occurring immunoglobulin molecules with varying structures. For example, native immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The subunit structures and three-dimensional configurations of the different classes of immunoglobulins are well known and described generally, for example, in Abbas et al., 2000. Cellular and Mol, and Kindt et al., Kuby Immunology. 6th ed., W.H. Freeman and Co., page 91 (2007). Antibodies (immunoglobulins) are assigned to different classes, depending on the amino acid sequences of the heavy chain constant domains. There are five major classes of antibodies: α (IgA), δ (IgD), ϵ (IgE), γ (IgG), or μ (IgM), some of which may be further divided into subtypes, e.g., γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

In some embodiments, an Fc, Fc region, or Fc domain refers to the C-terminal region of an antibody heavy chain that contains at least a portion of the constant region. The term includes native sequence Fc regions and variant Fc regions. An Fc can refer to the last two constant region immunoglobulin domains (e.g., CH2 and CH3) of IgA, IgD, and IgG, the last three constant region immunoglobulin domains of IgE and IgM, and optionally, all or a portion of the flexible hinge N-terminal to these domains. For IgA and IgM, Fc may include the J chain. An IgG Fc region comprises an IgG CH2 and an IgG CH3 domain and in some cases, inclusive of the hinge. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service. National Institutes of Health, Bethesda, Md., 1991. Human IgG Fc domains are of particular use in the present disclosure, and can be the Fc domain from human IgG1, IgG2 or IgG4.

As is known in the art, the C-terminal lysine of some antibody heavy chain polypeptides may be cleaved off in some fraction of molecules. Therefore, in some embodiments, an arm or multispecific binding molecule of the present disclosure may be present (e.g., in a composition) comprising a mixture of species in which some polypeptides retain the C-terminal lysine and some do not.

As is known in the art, variable domains of the heavy chain and light chain (VH and VL, respectively) of an antibody generally have similar structures, with each domain comprising four conserved framework regions (FRs) and three complementarity-determining regions (CDRs). (See, e.g., Kindt et al. Kuby Immunology, 6^{th} ed., W.H. Freeman and Co., page 91 (2007).) Framework (or “FR” as used herein) can refer to variable domain residues other than the CDR residues. The FR of a variable domain generally consists of four FR domains: FR1, FR2, FR3, and FR4. Accordingly, the CDR and FR sequences generally appear in the following sequence in VH (or VL): FR1-CDR1-FR2-CDR2-FR3-CDR3-FR4. In some embodiments, a FR1, FR2, FR3, and/or FR4 of the present disclosure refers to a human framework region, i.e., of the VH or VL domain.

Multiple definitions for the CDR sequences of antibody variable domains are known in the art; see, e.g., Kabat (Sequences of Proteins of Immunological Interest, Fifth Edition, NIH Publication 91-3242, Bethesda MD (1991), vols. 1-3) and Chothia. Unless otherwise specified, CDR sequences are described herein according to the definition of IMGT. See, e.g., imgt.org/IMGTScientificChart/Nomenclature/IMGT-FRCDRdefinition.html.

Examples of antibody fragments include, but are not limited to, Fab, Fab′, F(ab′)2, and Fv fragments, Fab′-SH, F(ab′)2, diabodies, linear antibodies, single chain antibodies, nanobodies, scFv fragments, VH, and multispecific (e.g., bispecific) antibodies/fragments formed from antibody fragments.

A “Fab” (fragment antigen binding) is a portion of an antibody that binds to antigens and includes the variable region and CH1 of the heavy chain linked to the light chain via an inter-chain disulfide bond.

### I. Multispecific Binding Proteins

In certain aspects, the present disclosure provides multispecific (e.g., bispecific) binding molecules comprising a first arm comprising a first antigen binding domain that binds to human Dectin-1; and a second arm comprising a second antigen binding domain that binds to human Trop-2. In some embodiments, the first and/or second antigen binding domain comprises a heavy chain variable (VH) domain and a light chain variable (VL) domain. In some embodiments, the second arm comprises an antibody heavy chain polypeptide (i.e., a second antibody heavy chain polypeptide of the present disclosure) and an antibody light chain polypeptide (i.e., a second antibody light chain polypeptide of the present disclosure). In some embodiments, the first arm comprises an antibody heavy chain polypeptide (i.e., a first antibody heavy chain polypeptide of the present disclosure) and an antibody light chain polypeptide (i.e., a first antibody light chain polypeptide of the present disclosure). In some embodiments, the first arm comprises a single chain variable fragment (scFv) linked to an Fc region.

Any one of the anti-Dectin-1 antigen binding domains or arms disclosed herein may be used in a binding molecule of the present disclosure. Any one of the anti-Trop-2 antigen binding domains or arms disclosed herein may be used in a binding molecule of the present disclosure. Various non-limiting embodiments of multispecific (e.g., bispecific) binding molecules of the present disclosure comprising specific combinations of an anti-Dectin-1 binding arm and an anti-Trop-2 binding arm are described in further detail infra.

### Anti-Dectin-1 Antigen Binding Domains

Various anti-Dectin-1 antigen binding domains are contemplated for use in the multispecific (e.g., bispecific) binding molecules of the present disclosure. In some embodiments, the anti-Dectin-1 antigen binding domain is the antigen binding domain of anti-Dectin-1 antibody 2M24, which is described in International Pub. No. WO2022077006, including variants thereof. In some embodiments, the anti-Dectin-1 antigen binding domain is a human antigen binding domain. In some embodiments, the anti-Dectin-1 antigen binding domain is a humanized antigen binding domain.

In some embodiments, the anti-Dectin-1 antigen binding domain, anti-Dectin-1 arm, or multispecific binding protein comprises a heavy chain variable (VH) domain and a light chain variable (VL) domain; wherein the VH domain comprises: a CDR-H1 comprising the amino acid sequence DYYI (SEQ ID NO: 1); a CDR-H2 comprising the amino acid sequence WINPNSGDTNYAQKFQG (SEQ ID NO: 2); and a CDR-H3 comprising the amino acid sequence of NSGSYSFGY (SEQ ID NO: 3); and wherein the VL domain comprises: a CDR-L1 comprising the amino acid sequence RASQGISSWLA (SEQ ID NO: 4); a CDR-L2 comprising the amino acid sequence GASSLQS (SEQ ID NO: 5), and a CDR-L3 comprising the amino acid sequence of QQAYSFPFT (SEQ ID NO: 6).

In some embodiments, the anti-Dectin-1 antigen binding domain, anti-Dectin-1 arm, or multispecific binding protein comprises a heavy chain variable (VH) domain and a light chain variable (VL) domain; wherein the VH domain comprises: a CDR-H1 comprising the amino acid sequence GYTFTDYY (SEQ ID NO:7); a CDR-H2 comprising the amino acid sequence INPNSGDT (SEQ ID NO: 8); and a CDR-H3 comprising the amino acid sequence of ARNSGSYSFGY (SEQ ID NO: 9); and wherein the VL domain comprises: a CDR-L1 comprising the amino acid sequence QGISSW (SEQ ID NO: 10); a CDR-L2 comprising the amino acid sequence GAS, and a CDR-L3 comprising the amino acid sequence of QQAYSFPFT (SEQ ID NO: 12).

In some embodiments, the anti-Dectin-1 antigen binding domain, anti-Dectin-1 arm, or multispecific binding protein comprises a heavy chain variable (VH) domain and a light chain variable (VL) domain; wherein the VH domain comprises: a CDR-H1 comprising the amino acid sequence GYTFTDY (SEQ ID NO:13); a CDR-H2 comprising the amino acid sequence NPNSGD (SEQ ID NO: 14); and a CDR-H3 comprising the amino acid sequence of NSGSYSFGY (SEQ ID NO: 15); and wherein the VL domain comprises: a CDR-L1 comprising the amino acid sequence RASQGISSWLA (SEQ ID NO: 4); a CDR-L2 comprising the amino acid sequence GASSLQS (SEQ ID NO: 5), and a CDR-L3 comprising the amino acid sequence of QQAYSFPFT (SEQ ID NO: 6).

In some embodiments, the anti-Dectin-1 antigen binding domain, anti-Dectin-1 arm, or multispecific binding protein comprises a heavy chain variable (VH) domain and a light chain variable (VL) domain; wherein the VH domain comprises: a CDR-H1 comprising the amino acid sequence DYYM (SEQ ID NO: 16); a CDR-H2 comprising the amino acid sequence WINPNEGDTNYAQKFEG (SEQ ID NO: 17); and a CDR-H3 comprising the amino acid sequence of NTGAYSFGY (SEQ ID NO: 18); and wherein the VL domain comprises: a CDR-L1 comprising the amino acid sequence RASQGISSWLA (SEQ ID NO: 4); a CDR-L2 comprising the amino acid sequence GASDLQS (SEQ ID NO: 19), and a CDR-L3 comprising the amino acid sequence of QQAYGFPFT (SEQ ID NO: 20).

In some embodiments, the anti-Dectin-1 antigen binding domain, anti-Dectin-1 arm, or multispecific binding protein comprises a heavy chain variable (VH) domain and a light chain variable (VL) domain; wherein the VH domain comprises: a CDR-H1 comprising the amino acid sequence GYTFTDYY (SEQ ID NO:7); a CDR-H2 comprising the amino acid sequence INPNEGDT (SEQ ID NO: 21); and a CDR-H3 comprising the amino acid sequence of ARNTGAYSFGY (SEQ ID NO: 22); and wherein the VL domain comprises: a CDR-L1 comprising the amino acid sequence QGISSW (SEQ ID NO: 10); a CDR-L2 comprising the amino acid sequence GAS, and a CDR-L3 comprising the amino acid sequence of QQAYGFPFT (SEQ ID NO: 20).

In some embodiments, the anti-Dectin-1 antigen binding domain, anti-Dectin-1 arm, or multispecific binding protein comprises a heavy chain variable (VH) domain and a light chain variable (VL) domain; wherein the VH domain comprises: a CDR-H1 comprising the amino acid sequence GYTFTDY (SEQ ID NO:13): a CDR-H2 comprising the amino acid sequence NPNEGD (SEQ ID NO: 23); and a CDR-H3 comprising the amino acid sequence of NTGAYSFGY (SEQ ID NO: 18); and wherein the VL domain comprises: a CDR-L1 comprising the amino acid sequence RASQGISSWLA (SEQ ID NO: 4); a CDR-L2 comprising the amino acid sequence GASDLQS (SEQ ID NO: 19), and a CDR-L3 comprising the amino acid sequence of QQAYGFPFT (SEQ ID NO: 20).

In some embodiments, the anti-Dectin-1 antigen binding domain, anti-Dectin-1 arm, or multispecific binding protein comprises a heavy chain variable (VH) domain and a light chain variable (VL) domain; wherein the VH domain comprises the amino acid sequence QVQLVQSGAEVKKPGASVKVSCKSSGYTFTDYYIHWVRQAPGQGLEWMGWINPNSGDTNYAQ KFQGRITMTRDTSISTAYLELSRLRSDDTAVFYCARNSGSYSFGYWGQGTLVTVSS (SEQ ID NO: 24) and/or wherein the VL domain comprises the amino acid sequence DIQMTQSPSSVSASVGDRVTITCRASQGISSWLAWYQQKPGKAPKLLIFGASSLQSGVPSRFSGSG SGTDFTLTVSSLQPEDFATYYCQQAYSFPFTFGPGTKVDIE (SEQ ID NO:25). In some embodiments, the VH domain comprises the amino acid sequence QVQLVQSGAEVKKPGASVKVSCKSSGYTFTDYYIHWVRQAPGQGLEWMGWINPNSGDTNYAQ KFQGRITMTRDTSISTAYLELSRLRSDDTAVFYCARNSGSYSFGYWGQGTLVTVSS (SEQ ID NO: 24) and the VL domain comprises the amino acid sequence

(SEQ ID NO: 25)
DIQMTQSPSSVSASVGDRVTITCRASQGISSWLAWYQQKPGKAPKLLIFG
ASSLQSGVPSRFSGSGSGTDFTLTVSSLQPEDFATYYCQQAYSFPFTFGP
GTKVDIE.

In some embodiments, the anti-Dectin-1 antigen binding domain, anti-Dectin-1 arm, or multispecific binding protein comprises a heavy chain variable (VH) domain and a light chain variable (VL) domain; wherein the VH domain comprises the amino acid sequence QVQLVQSGAEVKKPGASVKVSCKASGYTFTDYYMHWVRQAPGQGLEWMGWINPNEGDTNYA QKFEGRITMTRDTSISTAYMELSRLRSDDTAVYYCARNTGAYSFGYWGQGTLVTVSS (SEQ ID NO: 26) and/or wherein the VL domain comprises the amino acid sequence DIQMTQSPSSVSASVGDRVTITCRASQGISSWLAWYQQKPGKAPKLLIYGASDLQSGVPSRFSGS GSGTDFTLTISSLQPEDFATYYCQQAYGFPFTFGPGTKVDIK (SEQ ID NO:27). In some embodiments, the VH domain comprises the amino acid sequence QVQLVQSGAEVKKPGASVKVSCKASGYTFTDYYMHWVRQAPGQGLEWMGWINPNEGDTNYA QKFEGRITMTRDTSISTAYMELSRLRSDDTAVYYCARNTGAYSFGYWGQGTLVTVSS (SEQ ID NO: 26) and the VL domain comprises the amino acid sequence

(SEQ ID NO: 27)
DIQMTQSPSSVSASVGDRVTITCRASQGISSWLAWYQQKPGKAPKLLIYG
ASDLQSGVPSRFSGSGSGTDFTLTISSLQPEDFATYYCQQAYGFPFTFGP
GTKVDIK.

In some embodiments, the anti-Dectin-1 antigen binding domain, anti-Dectin-1 arm, or multispecific binding protein comprises a heavy chain variable (VH) domain and a light chain variable (VL) domain; wherein the VH domain comprises the amino acid sequence QVQLVQSGAEVKKPGASVKVSCKASGYTFTDYYMHWVRQAPGQGLEWMGWINPNEGDTNYA QKFEGRITMTRDTSISTAYMELSRLRSDDTAVYYCARNTGAYSFGYWGCGTLVTVSS (SEQ ID NO: 30) and/or wherein the VL domain comprises the amino acid sequence DIQMTQSPSSVSASVGDRVTITCRASQGISSWLAWYQQKPGKCPKLLIYGASDLQSGVPSRFSGS GSGTDFTLTISSLQPEDFATYYCQQAYGFPFTFGPGTKVDIK (SEQ ID NO:31). In some embodiments, the VH domain comprises the amino acid sequence QVQLVQSGAEVKKPGASVKVSCKASGYTFTDYYMHWVRQAPGQGLEWMGWINPNEGDTNYA QKFEGRITMTRDTSISTAYMELSRLRSDDTAVYYCARNTGAYSFGYWGCGTLVTVSS (SEQ ID NO: 30) and the VL domain comprises the amino acid sequence

(SEQ ID NO: 31)
DIQMTQSPSSVSASVGDRVTITCRASQGISSWLAWYQQKPGKCPKLLIYG
ASDLQSGVPSRFSGSGSGTDFTLTISSLQPEDFATYYCQQAYGFPFTFGP
GTKVDIK.

In some embodiments, an anti-Dectin-1 antigen binding domain, anti-Dectin-1 arm, or multispecific binding protein of the disclosure comprises a VH domain comprising 1, 2, or 3 CDRs from a single VH domain listed in Table 5 and/or a VL domain comprising 1, 2, or 3 CDRs from a single VL domain listed in Table 5. In some embodiments, an anti-Dectin-1 antigen binding domain, anti-Dectin-1 arm, or multispecific binding protein of the disclosure comprises a VH domain comprising 1, 2, or 3 CDRs from a single VH domain listed in Table 6 and/or a VL domain comprising 1, 2, or 3 CDRs from a single VL domain listed in Table 6. In some embodiments, an anti-Dectin-1 antigen binding domain, anti-Dectin-1 arm, or multispecific binding protein of the disclosure comprises a VH and/or VL domain listed in Table 6. In some embodiments, any set of 3 VH CDRs shown in Table 5 can be combined with any set of 3 VL CDRs shown in Table 5 in an anti-Dectin-1 antigen binding domain, anti-Dectin-1 arm, or multispecific binding protein of the present disclosure. In some embodiments, any set of 3 CDRs from a VH domain shown in Table 6 can be combined with any set of 3 CDRs from a VL domain shown in Table 6 in an anti-Dectin-1 antigen binding domain, anti-Dectin-1 arm, or multispecific binding protein of the present disclosure. In some embodiments, any VH domain shown in Table 6 can be combined with any VL domain shown in Table 6 in an anti-Dectin-1 antigen binding domain, anti-Dectin-1 arm, or multispecific binding protein of the present disclosure.

TABLE 5
Anti-Dectin-1 antibody CDR sequences

| Name | CDR-H1 | CDR-H2 | CDR-H3 | CDR-L1 | CDR-L2 | CDR-L3 |
|---|---|---|---|---|---|---|
| 2M24 | DYYI | WINPNSG | NSGSYSFG | RASQGIS | GASSLQS | QQAYSFPFT |
| (Kabat) | (SEQ ID | DTNYAQ | Y (SEQ ID | SWLA | (SEQ ID NO: | (SEQ ID NO: |
| | NO: 1) | KFQG | NO: 3) | (SEQ ID | 5) | 6 |
| | | (SEQ ID | | NO: 4) | | |
| | | NO: 2) | | | | |
| 2M24 | GYTFTDY | INPNSGD | ARNSGSYS | QGISSW | GAS | QQAYSFPFT |
| (IMGT) | Y (SEQ ID | T (SEQ ID | FGY (SEQ | (SEQ ID | | (SEQ ID NO: |
| | NO: 7) | NO: 8) | ID NO: 9) | NO: 10) | | 12) |
| 2M24 | GYTFTDY | NPNSGD | NSGSYSFG | RASQGIS | GASSLQS | QQAYSFPFT |
| (Chothia) | (SEQ ID | (SEQ ID | Y (SEQ ID | SWLA | (SEQ ID NO: | (SEQ ID NO: |
| | NO: 13) | NO: 14) | NO: 15) | (SEQ ID | 5) | 6 |
| | | | | NO: 4) | | |
| 2M24.119/ | DYYM | WINPNEG | NTGAYSFG | RASQGIS | GASDLQS | QQAYGFPFT |
| 124 | (SEQ ID | DTNYAQ | Y (SEQ ID | SWLA | (SEQ ID NO: | (SEQ ID NO: |
| | (Kabat) | NO: 16) | NO: 18) | (SEQ ID | 19) | 20) |
| | | (SEQ ID | | NO: 4) | | |
| | | NO: 17) | | | | |
| 2M24.119/ | GYTFTDY | INPNEGD | ARNTGAYS | QGISSW | GAS | QQAYGFPFT |
| 124 | Y (SEQ ID | T (SEQ ID | FGY (SEQ | (SEQ ID | | (SEQ ID NO: |
| (IMGT) | NO: 7) | NO: 21) | ID NO: 22) | NO: 10) | | 20) |
| 2M24.119/ | GYTFTDY | NPNEGD | NTGAYSFG | RASQGIS | GASDLQS | QQAYGFPFT |
| 124 | (SEQ ID | (SEQ ID | Y (SEQ ID | SWLA | (SEQ ID NO: | (SEQ ID NO: |
| (Chothia) | NO: 13) | NO: 23) | NO: 18) | (SEQ ID | 19) | 20) |
| | | | | NO: 4) | | |

TABLE 6
Anti-Dectin-1 antibody variable domain sequences

| Name | VH | VL |
|---|---|---|
| 2M24 | QVQLVQSGAEVKKPGASVKVSCKSSGYTF | DIQMTQSPSSVSASVGDRVTITCRAS |
| | TDYYIHWVRQAPGQGLEWMGWINPNSGD | QGISSWLAWYQQKPGKAPKLLIFGA |
| | TNYAQKFQGRITMTRDTSISTAYLELSRLR | SSLQSGVPSRFSGSGSGTDFTLTVSSL |
| | SDDTAVFYCARNSGSYSFGYWGQGTLVT | QPEDFATYYCQQAYSFPFTFGPGTKV |
| | VSS (SEQ ID NO: 24) | DIE (SEQ ID NO: 25) |
| 2M24.119 | QVQLVQSGAEVKKPGASVKVSCKASGYT | DIQMTQSPSSVSASVGDRVTITCRAS |
| | FTDYYMHWVRQAPGQGLEWMGWINPNE | QGISSWLAWYQQKPGKAPKLLIYGA |
| | GDTNYAQKFEGRITMTRDTSISTAYMELS | SDLQSGVPSRFSGSGSGTDFTLTISSL |
| | RLRSDDTAVYYCARNTGAYSFGYWGQGT | QPEDFATYYCQQAYGFPFTFGPGTK |
| | LVTVSS (SEQ ID NO: 26) | VDIK (SEQ ID NO: 27) |
| 2M24.124 | QVQLVQSGAEVKKPGASVKVSCKASGYT | DIQMTQSPSSVSASVGDRVTITCRAS |
| | FTDYYMHWVRQAPGQGLEWMGWINPNE | QGISSWLAWYQQKPGKCPKLLIYGA |
| | GDTNYAQKFEGRITMTRDTSISTAYMELS | SDLQSGVPSRFSGSGSGTDFTLTISSL |
| | RLRSDDTAVYYCARNTGAYSFGYWGCGT | QPEDFATYYCQQAYGFPFTFGPGTK |
| | LVTVSS (SEQ ID NO: 30) | VDIK (SEQ ID NO: 31) |

In some embodiments, the anti-Dectin-1 antigen binding domain or arm comprises a single chain variable fragment (scFv) that comprises the first VH domain and the first VL domain. In some embodiments, the scFv comprises a linker between the first VH domain and the first VL domain. Linkers for creating antibody fusion proteins are known in the art. In some embodiments, the linker comprises, consists of, or consists essentially of, glycine and/or serine residues. In some embodiments, the linker is 15-20 amino acids in length. In some embodiments, the linker comprises the sequence GGGSGGGSGGGS (SEQ ID NO:92). In some embodiments, the linker comprises one or more repeats of the sequence GGGGS (SEQ ID NO:93). In some embodiments, the linker comprises the sequence GGGGSGGGGSGGGGS (SEQ ID NO:94) or GGGGSGGGGSGGGGSGGGGS (SEQ ID NO:95). Additional linker sequences are described in Chen, X, et al. (2013) Adv. Drug Deliv. Rev. 65:1357-1369. In some embodiments (e.g., in a scFv of the present disclosure), the scFv comprises one type of linker between the VH and VL domains, and another type of linker connecting the VL domain to the rest of the arm, e.g., to an Fc region. For example, in some embodiments, the linker between the VH and VL domains comprises glycine and/or serine residues, such as GGGSGGGSGGGS (SEQ ID NO:92), GGGGSGGGGSGGGGS (SEQ ID NO:94), GGGGSGGGGSGGGGSGGGGS (SEQ ID NO:95), or one or more repeats of the sequence GGGGS (SEQ ID NO:96); and/or the linker connecting the VL domain to the Fc region comprises EPKRSDKTHTCPPC (SEQ ID NO:97) or SATHTCPPC (SEQ ID NO:98). In some embodiments, the linker between the VH and VL domains comprises glycine and/or serine residues and is 15-20 amino acids in length.

In some embodiments, the scFv comprises the amino acid sequence of

(SEQ ID NO: 34)
QVQLVQSGAEVKKPGASVKVSCKASGYTFTDYYMHWVRQAPGQGLEWMGW
INPNEGDTNYAQKFEGRITMTRDTSISTAYMELSRLRSDDTAVYYCARNT
GAYSFGYWGCGTLVTVSSGGGGSGGGGSGGGGSGGGGSDIQMTQSPSSVS
ASVGDRVTITCRASQGISSWLAWYQQKPGKCPKLLIYGASDLQSGVPSRF
SGSGSGTDFTLTISSLQPEDFATYYCQQAYGFPFTFGPGTKVDIKEPK.

In some embodiments, the anti-Dectin-1 arm comprises a single chain variable fragment (scFv) that comprises the VH domain and the VL domain of an anti-Dectin-1 antigen binding domain of the present disclosure and an antibody Fc region. In some embodiments, the anti-Dectin-1 arm comprises the amino acid sequence of QVQLVQSGAEVKKPGASVKVSCKASGYTFTDYYMHWVRQAPGQGLEWMGWINPNEGDTNYA QKFEGRITMTRDTSISTAYMELSRLRSDDTAVYYCARNTGAYSFGYWGCGTLVTVSSGGGGSGG GGSGGGGSGGGGSDIQMTQSPSSVSASVGDRVTITCRASQGISSWLAWYQQKPGKCPKLLIYGAS DLQSGVPSRFSGSGSGTDFTLTISSLQPEDFATYYCQQAYGFPFTFGPGTKVDIKEPKRSDKTHTCP PCPAPELLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTLPPSREE MTKNQVSLWCLVKGFYPSDIAVEWESNGQPENNYKTTPPVLDSDGSFFLYSKLTVDKSRWQQG NVFSCSVMHEALHNHYTQKSLSLSPG (SEQ ID NO:35) or QVQLVQSGAEVKKPGASVKVSCKASGYTFTDYYMHWVRQAPGQGLEWMGWINPNEGDTNYA QKFEGRITMTRDTSISTAYMELSRLRSDDTAVYYCARNTGAYSFGYWGCGTLVTVSSGGGGSGG GGSGGGGSGGGGSDIQMTQSPSSVSASVGDRVTITCRASQGISSWLAWYQQKPGKCPKLLIYGAS DLQSGVPSRFSGSGSGTDFTLTISSLQPEDFATYYCQQAYGFPFTFGPGTKVDIKEPKRSDKTHTCP PCPAPELLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTLPPSREE MTKNQVSLWCLVKGFYPSDIAVEWESNGQPENNYKTTPPVLDSDGSFFLYSKLTVDKSRWQQG NVFSCSVMHEALHNHYTQKSLSLSPGK (SEQ ID NO:36). For example, in some embodiments, a composition comprising an arm or multispecific binding molecule of the present disclosure comprises a mixture of species in which some species comprise a scFv-Fc fusion comprising the amino acid sequence of SEQ ID NO:35, while other species comprise a scFv-Fc fusion comprising the amino acid sequence of SEQ ID NO:36. In some embodiments, the anti-Dectin-1 arm comprises the amino acid sequence of QVQLVQSGAEVKKPGASVKVSCKASGYTFTDYYMHWVRQAPGQGLEWMGWINPNEG DTNYAQKFEGRITMTRDTSISTAYMELSRLRSDDTAVYYCARNTGAYSFGYWGCGTLV TVSSGGGGSGGGGSGGGGSGGGGSDIQMTQSPSSVSASVGDRVTITCRASQGISSWLAW YQQKPGKCPKLLIYGASDLQSGVPSRFSGSGSGTDFTLTISSLQPEDFATYYCQQAYGFPF TFGPGTKVDIKEPKRSDKTHTCPPCPAPELLGGPSVFLFPPKPKDTLMISRTPEVTCVVVD VSHEDPEVKFNWYVDGVEVHNAKTKPREEQYNSTYRVVSVLTVLHQDWLNGKEYKCK VSNKALPAPIEKTISKAKGQPREPQVYTLPPSREEMTKNQVSLSCAVKGFYPSDIAVEWE SNGQPENNYKTTPPVLDSDGSFFLVSKLTVDKSRWQQGNVFSCSVMHEALHNHYTQKS LSLSPG (SEQ ID NO:103) or QVQLVQSGAEVKKPGASVK VSCKASGYTFTDYYMHWVRQAPGQGLEWMGWINPNEG DTNYAQKFEGRITMTRDTSISTAYMELSRLRSDDTAVYYCARNTGAYSFGYWGCGTLV TVSSGGGGSGGGGSGGGGSGGGGSDIQMTQSPSSVSASVGDRVTITCRASQGISSWLAW YQQKPGKCPKLLIYGASDLQSGVPSRFSGSGSGTDFTLTISSLQPEDFATYYCQQAYGFPF TFGPGTKVDIKEPKRSDKTHTCPPCPAPELLGGPSVFLFPPKPKDTLMISRTPEVTCVVVD VSHEDPEVKFNWYVDGVEVHNAKTKPREEQYNSTYRVVSVLTVLHQDWLNGKEYKCK VSNKALPAPIEKTISKAKGQPREPQVYTLPPSREEMTKNQVSLSCAVKGFYPSDIAVEWE SNGQPENNYKTTPPVLDSDGSFFLVSKLTVDKSRWQQGNVFSCSVMHEALHNHYTQKS LSLSPGK (SEQ ID NO: 104). For example, in some embodiments, a composition comprising an arm or multispecific binding molecule of the present disclosure comprises a mixture of species in which some species comprise a scFv-Fc fusion comprising the amino acid sequence of SEQ ID NO: 103, while other species comprise a scFv-Fc fusion comprising the amino acid sequence of SEQ ID NO: 104.

In some embodiments, the anti-Dectin-1 arm comprises an antibody heavy chain polypeptide that comprises the VH domain of an anti-Dectin-1 antigen binding domain of the present disclosure and an antibody light chain polypeptide that comprises the VL domain of an anti-Dectin-1 antigen binding domain of the present disclosure. In some embodiments, the antibody heavy chain polypeptide further comprises an Fc region. In some embodiments, the anti-Dectin-1 arm comprises an antibody heavy chain polypeptide comprising the amino acid sequence QVQLVQSGAEVKKPGASVKVSCKSSGYTFTDYYIHWVRQAPGQGLEWMGWINPNSGDTNYAQ KFQGRITMTRDTSISTAYLELSRLRSDDTAVFYCARNSGSYSFGYWGQGTLVTVSSASTKGPSVF PLAPSSKSTSGGTAALGCLVKDYFPEPVTVSWNSGALTSGVHTFPAVLQSSGLYSLSSVVTVPSSS LGTQTYICNVNHKPSNTKVDKKVEPKSCDKTHTCPPCPAPELLGGPSVFLFPPKPKDTLMISRTPE VTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLWCLVKGFYPSDIAVEWESNG QPENNYKTTPPVLDSDGSFFLYSKLTVDKSRWQQGNVFSCSVMHEALHNHYTQKSLSLSPG (SEQ ID NO:87) or QVQLVQSGAEVKKPGASVKVSCKSSGYTFTDYYIHWVRQAPGQGLEWMGWINPNSGDTNYAQ KFQGRITMTRDTSISTAYLELSRLRSDDTAVFYCARNSGSYSFGYWGQGTLVTVSSASTKGPSVF PLAPSSKSTSGGTAALGCLVKDYFPEPVTVSWNSGALTSGVHTFPAVLQSSGLYSLSSVVTVPSSS LGTQTYICNVNHKPSNTKVDKKVEPKSCDKTHTCPPCPAPELLGGPSVFLFPPKPKDTLMISRTPE VTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSREEMTKNQVSLWCLVKGFYPSDIAVEWESNG QPENNYKTTPPVLDSDGSFFLYSKLTVDKSRWQQGNVFSCSVMHEALHNHYTQKSLSLSPGK (SEQ ID NO:88) and an antibody light chain polypeptide comprising the amino acid sequence DIQMTQSPSSVSASVGDRVTITCRASQGISSWLAWYQQKPGKAPKLLIFGASSLQSGVPSRFSGSG SGTDFTLTVSSLQPEDFATYYCQQAYSFPFTFGPGTKVDIERTVAAPSVFIFPPSDEQLKSGTASVV CLLNNFYPREAKVQWKVDNALQSGNSQESVTEQDSKDSTYSLSSTLTLSKADYEKHKVYACEVT HQGLSSPVTKSFNRGEC (SEQ ID NO:89). For example, in some embodiments, a composition comprising an arm or multispecific binding molecule of the present disclosure comprises a mixture of species in which some species comprise an antibody heavy chain polypeptide comprising the amino acid sequence of SEQ ID NO:87 and an antibody light chain polypeptide comprising the amino acid sequence of SEQ ID NO:89, while other species comprise an antibody heavy chain polypeptide comprising the amino acid sequence of SEQ ID NO:88 and an antibody light chain polypeptide comprising the amino acid sequence of SEQ ID NO:89. In some embodiments, the anti-Dectin-1 arm comprises an antibody heavy chain polypeptide comprising the amino acid sequence QVQLVQSGAEVKKPGASVKVSCKASGYTFTDYYMHWVRQAPGQGLEWMGWINPNEGDTNYA QKFEGRITMTRDTSISTAYMELSRLRSDDTAVYYCARNTGAYSFGYWGCGTLVTVSSASTKGPS VFPLAPSSKSTSGGTAALGCLVKDYFPEPVTVSWNSGALTSGVHTFPAVLQSSGLYSLSSVVTVPS SSLGTQTYICNVNHKPSNTKVDKKVEPKSSDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKE YKCKVSNKALPAPIEKTISKAKGQPREPQVYTLPPSREEMTKNQVSLWCLVKGFYPSDIAVEWES NGQPENNYKTTPPVLDSDGSFFLYSKLTVDKSRWQQGNVFSCSVMHEALHNHYTQKSLSLSPG (SEQ ID NO:32) or QVQLVQSGAEVKKPGASVKVSCKASGYTFTDYYMHWVRQAPGQGLEWMGWINPNEGDTNYA QKFEGRITMTRDTSISTAYMELSRLRSDDTAVYYCARNTGAYSFGYWGCGTLVTVSSASTKGPS VFPLAPSSKSTSGGTAALGCLVKDYFPEPVTVSWNSGALTSGVHTFPAVLQSSGLYSLSSVVTVPS SSLGTQTYICNVNHKPSNTKVDKKVEPKSSDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKE YKCKVSNKALPAPIEKTISKAKGQPREPQVYTLPPSREEMTKNQVSLWCLVKGFYPSDIAVEWES NGQPENNYKTTPPVLDSDGSFFLYSKLTVDKSRWQQGNVFSCSVMHEALHNHYTQKSLSLSPGK (SEQ ID NO:91) and an antibody light chain polypeptide comprising the amino acid sequence DIQMTQSPSSVSASVGDRVTITCRASQGISSWLAWYQQKPGKCPKLLIYGASDLQSGVPSRFSGS GSGTDFTLTISSLQPEDFATYYCQQAYGFPFTFGPGTKVDIKRTVAAPSVFIFPPSDEQLKSGTASV VCLLNNFYPREAKVQWKVDNALQSGNSQESVTEQDSKDSTYSLSSTLTLSKADYEKHKVYACE VTHQGLSSPVTKSFNRGES (SEQ ID NO:33). For example, in some embodiments, a composition comprising an arm or multispecific binding molecule of the present disclosure comprises a mixture of species in which some species comprise an antibody heavy chain polypeptide comprising the amino acid sequence of SEQ ID NO:32 and an antibody light chain polypeptide comprising the amino acid sequence of SEQ ID NO:33, while other species comprise an antibody heavy chain polypeptide comprising the amino acid sequence of SEQ ID NO:91 and an antibody light chain polypeptide comprising the amino acid sequence of SEQ ID NO:33. In some embodiments, the anti-Dectin-1 arm comprises an antibody heavy chain polypeptide comprising the amino acid sequence QVQLVQSGAEVKKPGASVKVSCKASGYTFTDYYMHWVRQAPGQGLEWMGWINPNEGDTNYA QKFEGRITMTRDTSISTAYMELSRLRSDDTAVYYCARNTGAYSFGYWGQGTLVTVSSASTKGPS VFPLAPSSKSTSGGTAALGCLVKDYFPEPVTVSWNSGALTSGVHTFPAVLQSSGLYSLSSVVTVPS SSLGTQTYICNVNHKPSNTKVDKKVEPKSCDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKE YKCKVSNKALPAPIEKTISKAKGQPREPQVYTLPPSREEMTKNQVSLWCLVKGFYPSDIAVEWES NGQPENNYKTTPPVLDSDGSFFLYSKLTVDKSRWQQGNVFSCSVMHEALHNHYTQKSLSLSPG (SEQ ID NO:28) or QVQLVQSGAEVKKPGASVKVSCKASGYTFTDYYMHWVRQAPGQGLEWMGWINPNEGDTNYA QKFEGRITMTRDTSISTAYMELSRLRSDDTAVYYCARNTGAYSFGYWGQGTLVTVSSASTKGPS VFPLAPSSKSTSGGTAALGCLVKDYFPEPVTVSWNSGALTSGVHTFPAVLQSSGLYSLSSVVTVPS SSLGTQTYICNVNHKPSNTKVDKKVEPKSCDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKE YKCKVSNKALPAPIEKTISKAKGQPREPQVYTLPPSREEMTKNQVSLWCLVKGFYPSDIAVEWES NGQPENNYKTTPPVLDSDGSFFLYSKLTVDKSRWQQGNVFSCSVMHEALHNHYTQKSLSLSPGK (SEQ ID NO:90) and an antibody light chain polypeptide comprising the amino acid sequence DIQMTQSPSSVSASVGDRVTITCRASQGISSWLAWYQQKPGKAPKLLIYGASDLQSGVPSRFSGS GSGTDFTLTISSLQPEDFATYYCQQAYGFPFTFGPGTKVDIKRTVAAPSVFIFPPSDEQLKSGTASV VCLLNNFYPREAKVQWKVDNALQSGNSQESVTEQDSKDSTYSLSSTLTLSKADYEKHKVYACE VTHQGLSSPVTKSFNRGEC (SEQ ID NO:29). For example, in some embodiments, a composition comprising an arm or multispecific binding molecule of the present disclosure comprises a mixture of species in which some species comprise an antibody heavy chain polypeptide comprising the amino acid sequence of SEQ ID NO:28 and an antibody light chain polypeptide comprising the amino acid sequence of SEQ ID NO:29, while other species comprise an antibody heavy chain polypeptide comprising the amino acid sequence of SEQ ID NO:90 and an antibody light chain polypeptide comprising the amino acid sequence of SEQ ID NO:29. In some embodiments, the anti-Dectin-1 arm comprises an antibody heavy chain polypeptide comprising the amino acid sequence QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKKVEPKSCDKTHTCPPCPAPELL GGPSVFLFPPKPKDTLMISRTPEVTCVVVDVSHEDPEVKFNWYVDGVEVHNAKTKPREE QYNSTYRVVSVLTVLHQDWLNGKEYKCKVSNKALPAPIEKTISKAKGQPREPQVYTLPP SREEMTKNQVSLSCAVKGFYPSDIAVEWESNGQPENNYKTTPPVLDSDGSFFLVSKLTV DKSRWQQGNVFSCSVMHEALHNHYTQKSLSLSPG (SEQ ID NO: 111) or QVQLVQSGAEVKKPGASVK VSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKKVEPKSCDKTHTCPPCPAPELL GGPSVFLFPPKPKDTLMISRTPEVTCVVVDVSHEDPEVKFNWYVDGVEVHNAKTKPREE QYNSTYRVVSVLTVLHQDWLNGKEYKCKVSNKALPAPIEKTISKAKGQPREPQVYTLPP SREEMTKNQVSLSCAVKGFYPSDIAVEWESNGQPENNYKTTPPVLDSDGSFFLVSKLTV DKSRWQQGNVFSCSVMHEALHNHYTQKSLSLSPGK (SEQ ID NO: 112) and an antibody light chain polypeptide comprising the amino acid sequence DIQMTQSPSSVSASVGDRVTITCRASQGISSWLAWYQQKPGKAPKLLIFGASSLQSGVPSRFSGSG SGTDFTLTVSSLQPEDFATYYCQQAYSFPFTFGPGTKVDIERTVAAPSVFIFPPSDEQLKSGTASVV CLLNNFYPREAKVQWKVDNALQSGNSQESVTEQDSKDSTYSLSSTLTLSKADYEKHKVYACEVT HQGLSSPVTKSFNRGEC (SEQ ID NO:89). For example, in some embodiments, a composition comprising an arm or multispecific binding molecule of the present disclosure comprises a mixture of species in which some species comprise an antibody heavy chain polypeptide comprising the amino acid sequence of SEQ ID NO: 111 and an antibody light chain polypeptide comprising the amino acid sequence of SEQ ID NO:89, while other species comprise an antibody heavy chain polypeptide comprising the amino acid sequence of SEQ ID NO: 112 and an antibody light chain polypeptide comprising the amino acid sequence of SEQ ID NO:89. In some embodiments, the anti-Dectin-1 arm comprises an antibody heavy chain polypeptide comprising the amino acid sequence QVQLVQSGAEVKKPGASVKVSCKASGYTFTDYYMHWVRQAPGQGLEWMGWINPNEG DTNYAQKFEGRITMTRDTSISTAYMELSRLRSDDTAVYYCARNTGAYSFGYWGCGTLV TVSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSSDKTHTCPPCPAPEL LGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLP PSREEMTKNQVSLSCAVKGFYPSDIAVEWESNGQPENNYKTTPPVLDSDGSFFLVSKLT VDKSRWQQGNVFSCSVMHEALHNHYTQKSLSLSPG (SEQ ID NO:101) or QVQLVQSGAEVKKPGASVKVSCKASGYTFTDYYMHWVRQAPGQGLEWMGWINPNEG DTNYAQKFEGRITMTRDTSISTAYMELSRLRSDDTAVYYCARNTGAYSFGYWGCGTLV TVSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSSDKTHTCPPCPAPEL LGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLP PSREEMTKNQVSLSCAVKGFYPSDIAVEWESNGQPENNYKTTPPVLDSDGSFFLVSKLT VDKSRWQQGNVFSCSVMHEALHNHYTQKSLSLSPGK (SEQ ID NO: 102) and an antibody light chain polypeptide comprising the amino acid sequence DIQMTQSPSSVSASVGDRVTITCRASQGISSWLAWYQQKPGKCPKLLIYGASDLQSGVPSRFSGS GSGTDFTLTISSLQPEDFATYYCQQAYGFPFTFGPGTKVDIKRTVAAPSVFIFPPSDEQLKSGTASV VCLLNNFYPREAKVQWKVDNALQSGNSQESVTEQDSKDSTYSLSSTLTLSKADYEKHKVYACE VTHQGLSSPVTKSFNRGES (SEQ ID NO:33). For example, in some embodiments, a composition comprising an arm or multispecific binding molecule of the present disclosure comprises a mixture of species in which some species comprise an antibody heavy chain polypeptide comprising the amino acid sequence of SEQ ID NO: 101 and an antibody light chain polypeptide comprising the amino acid sequence of SEQ ID NO:33, while other species comprise an antibody heavy chain polypeptide comprising the amino acid sequence of SEQ ID NO: 102 and an antibody light chain polypeptide comprising the amino acid sequence of SEQ ID NO:33. In some embodiments, the anti-Dectin-1 arm comprises an antibody heavy chain polypeptide comprising the amino acid sequence QVQLVQSGAEVKKPGASVKVSCKASGYTFTDYYMHWVRQAPGQGLEWMGWINPNEG DTNYAQKFEGRITMTRDTSISTAYMELSRLRSDDTAVYYCARNTGAYSFGYWGQGTLV TVSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPCPAPE LLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSREEMTKNQVSLSCAVKGFYPSDIAVEWESNGQPENNYKTTPPVLDSDGSFFLVSKLT VDKSRWQQGNVFSCSVMHEALHNHYTQKSLSLSPG (SEQ ID NO:99) or QVQLVQSGAEVKKPGASVKVSCKASGYTFTDYYMHWVRQAPGQGLEWMGWINPNEG DTNYAQKFEGRITMTRDTSISTAYMELSRLRSDDTAVYYCARNTGAYSFGYWGQGTLV TVSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPCPAPE LLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSREEMTKNQVSLSCAVKGFYPSDIAVEWESNGQPENNYKTTPPVLDSDGSFFLVSKLT VDKSRWQQGNVFSCSVMHEALHNHYTQKSLSLSPGK (SEQ ID NO: 100) and an antibody light chain polypeptide comprising the amino acid sequence DIQMTQSPSSVSASVGDRVTITCRASQGISSWLAWYQQKPGKAPKLLIYGASDLQSGVPSRFSGS GSGTDFTLTISSLQPEDFATYYCQQAYGFPFTFGPGTKVDIKRTVAAPSVFIFPPSDEQLKSGTASV VCLLNNFYPREAKVQWKVDNALQSGNSQESVTEQDSKDSTYSLSSTLTLSKADYEKHKVYACE VTHQGLSSPVTKSFNRGEC (SEQ ID NO:29). For example, in some embodiments, a composition comprising an arm or multispecific binding molecule of the present disclosure comprises a mixture of species in which some species comprise an antibody heavy chain polypeptide comprising the amino acid sequence of SEQ ID NO:99 and an antibody light chain polypeptide comprising the amino acid sequence of SEQ ID NO:29, while other species comprise an antibody heavy chain polypeptide comprising the amino acid sequence of SEQ ID NO: 100 and an antibody light chain polypeptide comprising the amino acid sequence of SEQ ID NO:29.

In some embodiments, the anti-Dectin-1 antigen binding domain, anti-Dectin-1 arm, or multispecific binding protein binds to human Dectin-1. In some embodiments, the anti-Dectin-1 antigen binding domain, anti-Dectin-1 arm, or multispecific binding protein binds to human Dectin-1 expressed on the surface of a macrophage, monocyte, dendritic cell, or granulocyte. In some embodiments, the anti-Dectin-1 antigen binding domain, anti-Dectin-1 arm, or multispecific binding protein binds to human Dectin-1 isoform A and/or human Dectin-1 isoform B. In some embodiments, human Dectin-1 isoform A comprises the amino acid sequence MEYHPDLENLDEDGYTQLHFDSQSNTRIAVVSEKGSCAASPPWRLIAVILGILCLVILVIAVVLGT MAIWRSNSGSNTLENGYFLSRNKENHSQPTQSSLEDSVTPTKAVKTTGVLSSPCPPNWIIYEKSCY LFSMSLNSWDGSKRQCWQLGSNLLKIDSSNELGFIVKQVSSQPDNSFWIGLSRPQTEVPWLWED GSTFSSNLFQIRTTATQENPSPNCVWIHVSVIYDQLCSVPSYSICEKKFSM (SEQ ID NO:84). In some embodiments, human Dectin-1 isoform B comprises the amino acid sequence

(SEQ ID NO: 85)
MEYHPDLENLDEDGYTQLHFDSQSNTRIAVVSEKGSCAASPPWRLIAVIL
GILCLVILVIAVVLGTMGVLSSPCPPNWIIYEKSCYLFSMSLNSWDGSKR
QCWQLGSNLLKIDSSNELGFIVKQVSSQPDNSFWIGLSRPQTEVPWLWED
GSTFSSNLFQIRTTATQENPSPNCVWIHVSVIYDQLCSVPSYSICEKKFS
M.

In some embodiments, the anti-Dectin-1 antigen binding domain, anti-Dectin-1 arm, or multispecific binding protein binds to human Dectin-1 expressed on the surface of a cell with an EC50 of less than 5 nM, less than 2 nM, less than InM, or less than 0.5 nM. In some embodiments, the anti-Dectin-1 antigen binding domain, anti-Dectin-1 arm, or multispecific binding protein is capable of binding to human Dectin-1 and monkey Dectin-1, e.g., cynomolgus Dectin-1.

### Anti-Trop-2 Antigen Binding Domains

Various anti-Trop-2 antigen binding domains are contemplated for use in the multispecific (e.g., bispecific) binding molecules of the present disclosure. In some embodiments, the anti-Trop-2 antigen binding domain is a human antigen binding domain. In some embodiments, the anti-Trop-2 antigen binding domain is a humanized antigen binding domain.

In some embodiments, the anti-Trop-2 antigen binding domain, anti-Trop-2 arm, or multispecific binding protein comprises a heavy chain variable (VH) domain and a light chain variable (VL) domain; wherein the VH domain comprises: a CDR-H1 comprising the amino acid sequence SSNWWS (SEQ ID NO: 37); a CDR-H2 comprising the amino acid sequence EIYHDGSTDYFPSLKS (SEQ ID NO: 38); and a CDR-H3 comprising the amino acid sequence of DNWGFDY (SEQ ID NO: 39); and wherein the VL domain comprises: a CDR-L1 comprising the amino acid sequence RASQSVSSSYL (SEQ ID NO: 40); a CDR-L2 comprising the amino acid sequence GASSRAT (SEQ ID NO: 41), and a CDR-L3 comprising the amino acid sequence of QQYGSSHRT (SEQ ID NO: 42).

In some embodiments, the anti-Trop-2 antigen binding domain, anti-Trop-2 arm, or multispecific binding protein comprises a heavy chain variable (VH) domain and a light chain variable (VL) domain; wherein the VH domain comprises: a CDR-H1 comprising the amino acid sequence GGSISSSNW (SEQ ID NO: 43); a CDR-H2 comprising the amino acid sequence IYHDGST (SEQ ID NO: 44); and a CDR-H3 comprising the amino acid sequence of ARDNWGFDY (SEQ ID NO: 45); and wherein the VL domain comprises: a CDR-L1 comprising the amino acid sequence QSVSSSY (SEQ ID NO: 46); a CDR-L2 comprising the amino acid sequence GAS, and a CDR-L3 comprising the amino acid sequence of QQYGSSHRT (SEQ ID NO: 42).

In some embodiments, the anti-Trop-2 antigen binding domain, anti-Trop-2 arm, or multispecific binding protein comprises a heavy chain variable (VH) domain and a light chain variable (VL) domain; wherein the VH domain comprises: a CDR-H1 comprising the amino acid sequence GGSISSSN (SEQ ID NO: 48); a CDR-H2 comprising the amino acid sequence YHDGS (SEQ ID NO: 49); and a CDR-H3 comprising the amino acid sequence of NWGFD (SEQ ID NO: 50); and wherein the VL domain comprises: a CDR-L1 comprising the amino acid sequence SQSVSSSY (SEQ ID NO: 51); a CDR-L2 comprising the amino acid sequence GAS, and a CDR-L3 comprising the amino acid sequence of YGSSHR (SEQ ID NO: 52).

In some embodiments, the anti-Trop-2 antigen binding domain, anti-Trop-2 arm, or multispecific binding protein comprises a heavy chain variable (VH) domain and a light chain variable (VL) domain; wherein the VH domain comprises: a CDR-H1 comprising the amino acid sequence SYGMN (SEQ ID NO: 53); a CDR-H2 comprising the amino acid sequence WINTNTGNPTYAQGFTG (SEQ ID NO: 54); and a CDR-H3 comprising the amino acid sequence of GYNWNDGDFDY (SEQ ID NO: 55); and wherein the VL domain comprises: a CDR-L1 comprising the amino acid sequence RASQGIRNDLG (SEQ ID NO: 56); a CDR-L2 comprising the amino acid sequence AASSLQS (SEQ ID NO: 57), and a CDR-L3 comprising the amino acid sequence of LQNYNYPLT (SEQ ID NO: 58).

In some embodiments, the anti-Trop-2 antigen binding domain, anti-Trop-2 arm, or multispecific binding protein comprises a heavy chain variable (VH) domain and a light chain variable (VL) domain; wherein the VH domain comprises: a CDR-H1 comprising the amino acid sequence GYTFTSYG (SEQ ID NO: 59); a CDR-H2 comprising the amino acid sequence INTNTGNP (SEQ ID NO: 60); and a CDR-H3 comprising the amino acid sequence of ARGYNWNDGDFDY (SEQ ID NO: 61); and wherein the VL domain comprises: a CDR-L1 comprising the amino acid sequence QGIRND (SEQ ID NO: 62); a CDR-L2 comprising the amino acid sequence AAS, and a CDR-L3 comprising the amino acid sequence of LQNYNYPLT (SEQ ID NO: 58).

In some embodiments, the anti-Trop-2 antigen binding domain, anti-Trop-2 arm, or multispecific binding protein comprises a heavy chain variable (VH) domain and a light chain variable (VL) domain; wherein the VH domain comprises: a CDR-H1 comprising the amino acid sequence GYTFTSY (SEQ ID NO: 64); a CDR-H2 comprising the amino acid sequence NTNTGN (SEQ ID NO: 65); and a CDR-H3 comprising the amino acid sequence of YNWNDGDFD (SEQ ID NO: 66); and wherein the VL domain comprises: a CDR-L1 comprising the amino acid sequence SQGIRND (SEQ ID NO: 67); a CDR-L2 comprising the amino acid sequence AAS, and a CDR-L3 comprising the amino acid sequence of NYNYPL (SEQ ID NO: 68).

In some embodiments, the anti-Trop-2 antigen binding domain, anti-Trop-2 arm, or multispecific binding protein comprises a heavy chain variable (VH) domain and a light chain variable (VL) domain; wherein the VH domain comprises the amino acid sequence QVQLQQSGSELKKPGASVKVSCKASGYTFTNYGMNWVKQAPGQGLKWMGWINTYTGEPTYTD DFKGRFAFSLDTSVSTAYLQISSLKADDTAVYFCARGGFGSSYWYFDVWGQGSLVTVSS (SEQ ID NO: 69) and/or wherein the VL domain comprises the amino acid sequence DIQLTQSPSSLSASVGDRVSITCKASQDVSIAVAWYQQKPGKAPKLLIYSASYRYTGVPDRFSGSG SGTDFTLTISSLQPEDFAVYYCQQHYITPLTFGAGTKVEIK (SEQ ID NO:70). In some embodiments, the VH domain comprises the amino acid sequence QVQLQQSGSELKKPGASVKVSCKASGYTFTNYGMNWVKQAPGQGLKWMGWINTYTGEPTYTD DFKGRFAFSLDTSVSTAYLQISSLKADDTAVYFCARGGFGSSYWYFDVWGQGSLVTVSS (SEQ ID NO: 69) and the VL domain comprises the amino acid sequence

(SEQ ID NO: 70)
DIQLTQSPSSLSASVGDRVSITCKASQDVSIAVAWYQQKPGKAPKLLIYS
ASYRYTGVPDRFSGSGSGTDFTLTISSLQPEDFAVYYCQQHYITPLTFGA
GTKVEIK.

In some embodiments, the anti-Trop-2 antigen binding domain, anti-Trop-2 arm, or multispecific binding protein comprises a heavy chain variable (VH) domain and a light chain variable (VL) domain; wherein the VH domain comprises the amino acid sequence QVQLQESGPGLVKPSGTLSLTCAVSGGSISSSNWWSWVRQPPGKGLEWIGEIYHDGSTDYFPSLK SRVTISVDKSKNQFSLKLSSVTAADTAVYYCARDNWGFDYWGQGTLVTVSS (SEQ ID NO:74) and/or wherein the VL domain comprises the amino acid sequence DIVLTQSPGTLSLSPGERATLSCRASQSVSSSYLAWYQQKPGQAPRLLISGASSRATGIPDRFSGSG SGTDFTLTISRLEPEDFAVYYCQQYGSSHRTFGQGTKVEIK (SEQ ID NO:75). In some embodiments, the VH domain comprises the amino acid sequence QVQLQESGPGLVKPSGTLSLTCAVSGGSISSSNWWSWVRQPPGKGLEWIGEIYHDGSTDYFPSLK SRVTISVDKSKNQFSLKLSSVTAADTAVYYCARDNWGFDYWGQGTLVTVSS (SEQ ID NO: 74) and the VL domain comprises the amino acid sequence

(SEQ ID NO: 75)
DIVLTQSPGTLSLSPGERATLSCRASQSVSSSYLAWYQQKPGQAPRLLIS
GASSRATGIPDRESGSGSGTDFTLTISRLEPEDFAVYYCQQYGSSHRTFG
QGTKVEIK.

In some embodiments, the anti-Trop-2 antigen binding domain, anti-Trop-2 arm, or multispecific binding protein comprises a heavy chain variable (VH) domain and a light chain variable (VL) domain; wherein the VH domain comprises the amino acid sequence QVQLVQSGSELKKPGASVKLSCKASGYTFTSYGMNWVRQAPGQGLEWMGWINTNTGNPTYAQ GFTGRFVFSLDTSVSTAYLQISSLKAEDTAVYYCARGYNWNDGDFDYWGQGTLVTVSS (SEQ ID NO: 79) and/or wherein the VL domain comprises the amino acid sequence AIQMTQSPSSLSPSVGDRVTITCRASQGIRNDLGWYQQKPGKAPKLLIFAASSLQSGVPSRFSGSG SGTDFTLTISNLQPEDFATYYCLQNYNYPLTFGGGTKVEIK (SEQ ID NO:80). In some embodiments, the VH domain comprises the amino acid sequence QVQLVQSGSELKKPGASVKLSCKASGYTFTSYGMNWVRQAPGQGLEWMGWINTNTGNPTYAQ GFTGRFVFSLDTSVSTAYLQISSLKAEDTAVYYCARGYNWNDGDFDYWGQGTLVTVSS (SEQ ID NO: 79) and the VL domain comprises the amino acid sequence

(SEQ ID NO: 80)
AIQMTQSPSSLSPSVGDRVTITCRASQGIRNDLGWYQQKPGKAPKLLIFA
ASSLQSGVPSRFSGSGSGTDFTLTISNLQPEDFATYYCLQNYNYPLTFGG
GTKVEIK.

In some embodiments, an anti-Trop-2 antigen binding domain, anti-Trop-2 arm, or multispecific binding protein of the disclosure comprises a VH domain comprising 1, 2, or 3 CDRs from a single VH domain listed in Table 7 and/or a VL domain comprising 1, 2, or 3 CDRs from a single VL domain listed in Table 7. In some embodiments, an anti-Trop-2 antigen binding domain, anti-Trop-2 arm, or multispecific binding protein of the disclosure comprises a VH domain comprising 1, 2, or 3 CDRs from a single VH domain listed in Table 8 and/or a VL domain comprising 1, 2, or 3 CDRs from a single VL domain listed in Table 8. In some embodiments, an anti-Trop-2 antigen binding domain, anti-Trop-2 arm, or multispecific binding protein of the disclosure comprises a VH and/or VL domain listed in Table 8. In some embodiments, any set of 3 VH CDRs shown in Table 7 can be combined with any set of 3 VL CDRs shown in Table 7 in an anti-Trop-2 antigen binding domain, anti-Trop-2 arm, or multispecific binding protein of the present disclosure. In some embodiments, any set of 3 CDRs from a VH domain shown in Table 8 can be combined with any set of 3 CDRs from a VL domain shown in Table 8 in an anti-Trop-2 antigen binding domain, anti-Trop-2 arm, or multispecific binding protein of the present disclosure. In some embodiments, any VH domain shown in Table 8 can be combined with any VL domain shown in Table 8 in an anti-Trop-2 antigen binding domain, anti-Trop-2 arm, or multispecific binding protein of the present disclosure.

TABLE 7
Anti-Trop-2 antibody CDR sequences

| Name | CDR-H1 | CDR-H2 | CDR-H3 | CDR-L1 | CDR-L2 | CDR-L3 |
|---|---|---|---|---|---|---|
| DR02B.65 | SSNWWS | EIYHDGS | DNWGFDY | RASQSVS | GASSRAT | QQYGSSHRT |
| Clone B | (SEQ ID | TDYFPSL | (SEQ ID | SSYL | (SEQ ID | (SEQ ID |
| (Kabat) | NO: 37) | KS | NO: 39) | (SEQ ID | NO: 41) | NO: 42) |
| | | (SEQ ID | | NO: 40) | | |
| | | NO: 38) | | | | |
| DR02B.65 | GGSISSSN | IYHDGST | ARDNWGF | QSVSSSY | GAS | QQYGSSHRT |
| Clone B | W | (SEQ ID | DY | (SEQ ID | | (SEQ ID |
| (IMGT) | (SEQ ID | NO: 44) | (SEQ ID | NO: 46) | | NO: 42) |
| | NO: 43) | | NO: 45) | | | |
| DR02B.65 | GGSISSSN | YHDGS | NWGFD | SQSVSSS | GAS | YGSSHR |
| Clone B | (SEQ ID | (SEQ ID | (SEQ ID | Y | | (SEQ ID |
| (Chothia) | NO: 48) | NO: 49) | NO: 50) | (SEQ ID | | NO: 52) |
| | | | | NO: 51) | | |
| DR02B.77 | SYGMN | WINTNTG | GYNWNDG | RASQGIR | AASSLQS | LQNYNYPLT |
| Clone C | (SEQ ID | NPTYAQ | DFDY | NDLG | (SEQ ID | (SEQ ID |
| (Kabat) | NO: 53) | GFTG | (SEQ ID | (SEQ ID | NO: 57) | NO: 58) |
| | | (SEQ ID | NO: 55) | NO: 56) | | |
| | | NO: 54) | | | | |
| DR02B.77 | GYTFTSY | INTNTGN | ARGYNWN | QGIRND | AAS | LQNYNYPLT |
| Clone C | G | P | DGDFDY | (SEQ ID | | (SEQ ID |
| (IMGT) | (SEQ ID | (SEQ ID | (SEQ ID | NO: 62) | | NO: 58) |
| | NO: 59) | NO: 60) | NO: 61) | | | |
| DR02B.77 | GYTFTSY | NTNTGN | YNWNDGD | SQGIRND | AAS | NYNYPL |
| Clone C | (SEQ ID | (SEQ ID | FD (SEQ ID | (SEQ ID | | (SEQ ID |
| (Chothia) | NO: 64) | NO: 65) | NO: 66) | NO: 67) | | NO: 68) |

TABLE 8
Anti-Trop-2 antibody variable domain sequences

| Name | VH | VL |
|---|---|---|
| Clone | QVQLQQSGSELKKPGASVKVSCKASGYTF | DIQLTQSPSSLSASVGDRVSITCKASQ |
| A | TNYGMNWVKQAPGQGLKWMGWINTYT | DVSIAVAWYQQKPGKAPKLLIYSAS |
| | GEPTYTDDFKGRFAFSLDTSVSTAYLQISS | YRYTGVPDRFSGSGSGTDFTLTISSLQ |
| | LKADDTAVYFCARGGFGSSYWYFDVWG | PEDFAVYYCQQHYITPLTFGAGTKVE |
| | QGSLVTVSS (SEQ ID NO: 69) | IK (SEQ ID NO: 70) |
| Clone | QVQLQESGPGLVKPSGTLSLTCAVSGGSIS | DIVLTQSPGTLSLSPGERATLSCRASQ |
| B | SSNWWSWVRQPPGKGLEWIGEIYHDGST | SVSSSYLAWYQQKPGQAPRLLISGAS |
| | DYFPSLKSRVTISVDKSKNQFSLKLSSVTA | SRATGIPDRFSGSGSGTDFTLTISRLEP |
| | ADTAVYYCARDNWGFDYWGQGTLVTVS | EDFAVYYCQQYGSSHRTFGQGTKVE |
| | S (SEQ ID NO: 74) | IK (SEQ ID NO: 75) |
| Clone | QVQLVQSGSELKKPGASVKLSCKASGYTF | AIQMTQSPSSLSPSVGDRVTITCRASQ |
| C | TSYGMNWVRQAPGQGLEWMGWINTNTG | GIRNDLGWYQQKPGKAPKLLIFAASS |
| | NPTYAQGFTGRFVFSLDTSVSTAYLQISSL | LQSGVPSRFSGSGSGTDFTLTISNLQP |
| | KAEDTAVYYCARGYNWNDGDFDYWGQG | EDFATYYCLQNYNYPLTFGGGTKVE |
| | TLVTVSS (SEQ ID NO: 79) | IK (SEQ ID NO: 80) |

In some embodiments, the anti-Trop-2 arm comprises an antibody heavy chain polypeptide that comprises the VH domain of an anti-Trop-2 antigen binding domain of the present disclosure and an antibody light chain polypeptide that comprises the VL domain of an anti-Trop-2 antigen binding domain of the present disclosure. In some embodiments, the antibody heavy chain polypeptide further comprises an Fc region. In some embodiments, the anti-Trop-2 arm comprises an antibody heavy chain polypeptide comprising the amino acid sequence QVQLQQSGSELKKPGASVKVSCKASGYTFTNYGMNWVKQAPGQGLKWMGWINTYTGEPTYTD DFKGRFAFSLDTSVSTAYLQISSLKADDTAVYFCARGGFGSSYWYFDVWGQGSLVTVSSASTKG PSVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPSVFLFPPKPKDTLMIS RTPEVTCVVVDVSHEDPEVKFNWYVDGVEVHNAKTKPREEQYNSTYRVVSVLTVLHQDWLNG KEYKCKVSNKALPAPIEKTISKAKGQPREPQVYTLPPSREEMTKNQVSLSCAVKGFYPSDIAVEW ESNGQPENNYKTTPPVLDSDGSFFLVSKLTVDKSRWQQGNVFSCSVMHEALHNHYTQKSLSLSP G (SEQ ID NO:71) or QVQLQQSGSELKKPGASVKVSCKASGYTFTNYGMNWVKQAPGQGLKWMGWINTYTGEPTYTD DFKGRFAFSLDTSVSTAYLQISSLKADDTAVYFCARGGFGSSYWYFDVWGQGSLVTVSSASTKG PSVFPLAPSSKSTSGGTAALGCLVKDYFPEPVTVSWNSGALTSGVHTFPAVLQSSGLYSLSSVVTV PSSSLGTQTYICNVNHKPSNTKVDKKVEPKSCDKTHTCPPCPAPELLGGPSVFLFPPKPKDTLMIS RTPEVTCVVVDVSHEDPEVKFNWYVDGVEVHNAKTKPREEQYNSTYRVVSVLTVLHQDWLNG KEYKCKVSNKALPAPIEKTISKAKGQPREPQVYTLPPSREEMTKNQVSLSCAVKGFYPSDIAVEW ESNGQPENNYKTTPPVLDSDGSFFLVSKLTVDKSRWQQGNVFSCSVMHEALHNHYTQKSLSLSP GK (SEQ ID NO:72) and an antibody light chain polypeptide comprising the amino acid sequence DIQLTQSPSSLSASVGDRVSITCKASQDVSIAVAWYQQKPGKAPKLLIYSASYRYTGVPDRFSGSG SGTDFTLTISSLQPEDFAVYYCQQHYITPLTFGAGTKVEIKRTVAAPSVFIFPPSDEQLKSGTASVV CLLNNFYPREAKVQWKVDNALQSGNSQESVTEQDSKDSTYSLSSTLTLSKADYEKHKVYACEVT HQGLSSPVTKSFNRGEC (SEQ ID NO:73). For example, in some embodiments, a composition comprising an arm or multispecific binding molecule of the present disclosure comprises a mixture of species in which some species comprise an antibody heavy chain polypeptide comprising the amino acid sequence of SEQ ID NO:71 and an antibody light chain polypeptide comprising the amino acid sequence of SEQ ID NO:73, while other species comprise an antibody heavy chain polypeptide comprising the amino acid sequence of SEQ ID NO: 72 and an antibody light chain polypeptide comprising the amino acid sequence of SEQ ID NO:73. In some embodiments, the anti-Trop-2 arm comprises an antibody heavy chain polypeptide comprising the amino acid sequence QVQLQESGPGLVKPSGTLSLTCAVSGGSISSSNWWSWVRQPPGKGLEWIGEIYHDGSTDYFPSLK SRVTISVDKSKNQFSLKLSSVTAADTAVYYCARDNWGFDYWGQGTLVTVSSASTKGPSVFPLAP SSKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFPPKPKDTLMISRTPEVTC VVVDVSHEDPEVKFNWYVDGVEVHNAKTKPREEQYNSTYRVVSVLTVLHQDWLNGKEYKCK VSNKALPAPIEKTISKAKGQPREPQVYTLPPSREEMTKNQVSLSCAVKGFYPSDIAVEWESNGQPE NNYKTTPPVLDSDGSFFLVSKLTVDKSRWQQGNVFSCSVMHEALHNHYTQKSLSLSPG (SEQ ID NO: 76) or QVQLQESGPGLVKPSGTLSLTCAVSGGSISSSNWWSWVRQPPGKGLEWIGEIYHDGSTDYFPSLK SRVTISVDKSKNQFSLKLSSVTAADTAVYYCARDNWGFDYWGQGTLVTVSSASTKGPSVFPLAP SSKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFPPKPKDTLMISRTPEVTC VVVDVSHEDPEVKFNWYVDGVEVHNAKTKPREEQYNSTYRVVSVLTVLHQDWLNGKEYKCK VSNKALPAPIEKTISKAKGQPREPQVYTLPPSREEMTKNQVSLSCAVKGFYPSDIAVEWESNGQPE NNYKTTPPVLDSDGSFFLVSKLTVDKSRWQQGNVFSCSVMHEALHNHYTQKSLSLSPGK (SEQ ID NO: 77) and an antibody light chain polypeptide comprising the amino acid sequence DIVLTQSPGTLSLSPGERATLSCRASQSVSSSYLAWYQQKPGQAPRLLISGASSRATGIPDRFSGSG SGTDFTLTISRLEPEDFAVYYCQQYGSSHRTFGQGTKVEIKRTVAAPSVFIFPPSDEQLKSGTASVV CLLNNFYPREAKVQWKVDNALQSGNSQESVTEQDSKDSTYSLSSTLTLSKADYEKHKVYACEVT HQGLSSPVTKSFNRGEC (SEQ ID NO:78). For example, in some embodiments, a composition comprising an arm or multispecific binding molecule of the present disclosure comprises a mixture of species in which some species comprise an antibody heavy chain polypeptide comprising the amino acid sequence of SEQ ID NO: 76 and an antibody light chain polypeptide comprising the amino acid sequence of SEQ ID NO:78, while other species comprise an antibody heavy chain polypeptide comprising the amino acid sequence of SEQ ID NO: 77 and an antibody light chain polypeptide comprising the amino acid sequence of SEQ ID NO:78. In some embodiments, the anti-Trop-2 arm comprises an antibody heavy chain polypeptide comprising the amino acid sequence QVQLVQSGSELKKPGASVKLSCKASGYTFTSYGMNWVRQAPGQGLEWMGWINTNTGNPTYAQ GFTGRFVFSLDTSVSTAYLQISSLKAEDTAVYYCARGYNWNDGDFDYWGQGTLVTVSSASTKGP SVFPLAPSSKSTSGGTAALGCLVKDYFPEPVTVSWNSGALTSGVHTFPAVLQSSGLYSLSSVVTVP SSSLGTQTYICNVNHKPSNTKVDKKVEPKSCDKTHTCPPCPAPELLGGPSVFLFPPKPKDTLMISR TPEVTCVVVDVSHEDPEVKFNWYVDGVEVHNAKTKPREEQYNSTYRVVSVLTVLHQDWLNGK EYKCKVSNKALPAPIEKTISKAKGQPREPQVYTLPPSREEMTKNQVSLSCAVKGFYPSDIAVEWE SNGQPENNYKTTPPVLDSDGSFFLVSKLTVDKSRWQQGNVFSCSVMHEALHNHYTQKSLSLSPG (SEQ ID NO:81) or QVQLVQSGSELKKPGASVKLSCKASGYTFTSYGMNWVRQAPGQGLEWMGWINTNTGNPTYAQ GFTGRFVFSLDTSVSTAYLQISSLKAEDTAVYYCARGYNWNDGDFDYWGQGTLVTVSSASTKGP SVFPLAPSSKSTSGGTAALGCLVKDYFPEPVTVSWNSGALTSGVHTFPAVLQSSGLYSLSSVVTVP SSSLGTQTYICNVNHKPSNTKVDKKVEPKSCDKTHTCPPCPAPELLGGPSVFLFPPKPKDTLMISR TPEVTCVVVDVSHEDPEVKFNWYVDGVEVHNAKTKPREEQYNSTYRVVSVLTVLHQDWLNGK EYKCKVSNKALPAPIEKTISKAKGQPREPQVYTLPPSREEMTKNQVSLSCAVKGFYPSDIAVEWE SNGQPENNYKTTPPVLDSDGSFFLVSKLTVDKSRWQQGNVFSCSVMHEALHNHYTQKSLSLSPG K (SEQ ID NO:82) and an antibody light chain polypeptide comprising the amino acid sequence AIQMTQSPSSLSPSVGDRVTITCRASQGIRNDLGWYQQKPGKAPKLLIFAASSLQSGVPSRFSGSG SGTDFTLTISNLQPEDFATYYCLQNYNYPLTFGGGTKVEIKRTVAAPSVFIFPPSDEQLKSGTASV VCLLNNFYPREAKVQWKVDNALQSGNSQESVTEQDSKDSTYSLSSTLTLSKADYEKHKVYACE VTHQGLSSPVTKSFNRGEC (SEQ ID NO:83). For example, in some embodiments, a composition comprising an arm or multispecific binding molecule of the present disclosure comprises a mixture of species in which some species comprise an antibody heavy chain polypeptide comprising the amino acid sequence of SEQ ID NO:81 and an antibody light chain polypeptide comprising the amino acid sequence of SEQ ID NO:83, while other species comprise an antibody heavy chain polypeptide comprising the amino acid sequence of SEQ ID NO:82 and an antibody light chain polypeptide comprising the amino acid sequence of SEQ ID NO:83. In some embodiments, the anti-Trop-2 arm comprises an antibody heavy chain polypeptide comprising the amino acid sequence QVQLQQSGSELKKPGASVKVSCKASGYTFTNYGMNWVKQAPGQGLKWMGWINTYTG EPTYTDDFKGRFAFSLDTSVSTAYLQISSLKADDTAVYFCARGGFGSSYWYFDVWGQGS LVTVSSASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKKVEPKSCDKTHTCPPCPA PELLGGPSVFLFPPKPKDTLMISRTPEVTCVVVDVSHEDPEVKFNWYVDGVEVHNAKTK PREEQYNSTYRVVSVLTVLHQDWLNGKEYKCKVSNKALPAPIEKTISKAKGQPREPQVY TLPPSREEMTKNQVSLWCLVKGFYPSDIAVEWESNGQPENNYKTTPPVLDSDGSFFLYS KLTVDKSRWQQGNVFSCSVMHEALHNHYTQKSLSLSPG (SEQ ID NO: 105) or QVQLQQSGSELKKPGASVKVSCKASGYTFTNYGMNWVKQAPGQGLKWMGWINTYTG EPTYTDDFKGRFAFSLDTSVSTAYLQISSLKADDTAVYFCARGGFGSSYWYFDVWGQGS LVTVSSASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKKVEPKSCDKTHTCPPCPA PELLGGPSVFLFPPKPKDTLMISRTPEVTCVVVDVSHEDPEVKFNWYVDGVEVHNAKTK PREEQYNSTYRVVSVLTVLHQDWLNGKEYKCKVSNKALPAPIEKTISKAKGQPREPQVY TLPPSREEMTKNQVSLWCLVKGFYPSDIAVEWESNGQPENNYKTTPPVLDSDGSFFLYS KLTVDKSRWQQGNVFSCSVMHEALHNHYTQKSLSLSPGK (SEQ ID NO: 106) and an antibody light chain polypeptide comprising the amino acid sequence DIQLTQSPSSLSASVGDRVSITCKASQDVSIAVAWYQQKPGKAPKLLIYSASYRYTGVPDRFSGSG SGTDFTLTISSLQPEDFAVYYCQQHYITPLTFGAGTKVEIKRTVAAPSVFIFPPSDEQLKSGTASVV CLLNNFYPREAKVQWKVDNALQSGNSQESVTEQDSKDSTYSLSSTLTLSKADYEKHKVYACEVT HQGLSSPVTKSFNRGEC (SEQ ID NO:73). For example, in some embodiments, a composition comprising an arm or multispecific binding molecule of the present disclosure comprises a mixture of species in which some species comprise an antibody heavy chain polypeptide comprising the amino acid sequence of SEQ ID NO: 105 and an antibody light chain polypeptide comprising the amino acid sequence of SEQ ID NO:73, while other species comprise an antibody heavy chain polypeptide comprising the amino acid sequence of SEQ ID NO: 106 and an antibody light chain polypeptide comprising the amino acid sequence of SEQ ID NO:73. In some embodiments, the anti-Trop-2 arm comprises an antibody heavy chain polypeptide comprising the amino acid sequence QVQLQESGPGLVKPSGTLSLTCAVSGGSISSSNWWSWVRQPPGKGLEWIGEIYHDGSTD YFPSLKSRVTISVDKSKNQFSLKLSSVTAADTAVYYCARDNWGFDYWGQGTLVTVSSA STKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGP SVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR EEMTKNQVSLWCLVKGFYPSDIAVEWESNGQPENNYKTTPPVLDSDGSFFLYSKLTVDK SRWQQGNVFSCSVMHEALHNHYTQKSLSLSPG (SEQ ID NO: 107) or QVQLQESGPGLVKPSGTLSLTCAVSGGSISSSNWWSWVRQPPGKGLEWIGEIYHDGSTD YFPSLKSRVTISVDKSKNQFSLKLSSVTAADTAVYYCARDNWGFDYWGQGTLVTVSSA STKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGP SVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR EEMTKNQVSLWCLVKGFYPSDIAVEWESNGQPENNYKTTPPVLDSDGSFFLYSKLTVDK SRWQQGNVFSCSVMHEALHNHYTQKSLSLSPGK (SEQ ID NO: 108) and an antibody light chain polypeptide comprising the amino acid sequence DIVLTQSPGTLSLSPGERATLSCRASQSVSSSYLAWYQQKPGQAPRLLISGASSRATGIPDRFSGSG SGTDFTLTISRLEPEDFAVYYCQQYGSSHRTFGQGTKVEIKRTVAAPSVFIFPPSDEQLKSGTASVV CLLNNFYPREAKVQWKVDNALQSGNSQESVTEQDSKDSTYSLSSTLTLSKADYEKHKVYACEVT HQGLSSPVTKSFNRGEC (SEQ ID NO:78). For example, in some embodiments, a composition comprising an arm or multispecific binding molecule of the present disclosure comprises a mixture of species in which some species comprise an antibody heavy chain polypeptide comprising the amino acid sequence of SEQ ID NO: 107 and an antibody light chain polypeptide comprising the amino acid sequence of SEQ ID NO:78, while other species comprise an antibody heavy chain polypeptide comprising the amino acid sequence of SEQ ID NO: 108 and an antibody light chain polypeptide comprising the amino acid sequence of SEQ ID NO:78. In some embodiments, the anti-Trop-2 arm comprises an antibody heavy chain polypeptide comprising the amino acid sequence QVQLVQSGSELKKPGASVKLSCKASGYTFTSYGMNWVRQAPGQGLEWMGWINTNTGN PTYAQGFTGRFVFSLDTSVSTAYLQISSLKAEDTAVYYCARGYNWNDGDFDYWGQGTL VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCPPCPAP ELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYT LPPSREEMTKNQVSLWCLVKGFYPSDIAVEWESNGQPENNYKTTPPVLDSDGSFFLYSK LTVDKSRWQQGNVFSCSVMHEALHNHYTQKSLSLSPG (SEQ ID NO: 109) or QVQLVQSGSELKKPGASVKLSCKASGYTFTSYGMNWVRQAPGQGLEWMGWINTNTGN PTYAQGFTGRFVFSLDTSVSTAYLQISSLKAEDTAVYYCARGYNWNDGDFDYWGQGTL VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCPPCPAP ELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYT LPPSREEMTKNQVSLWCLVKGFYPSDIAVEWESNGQPENNYKTTPPVLDSDGSFFLYSK LTVDKSRWQQGNVFSCSVMHEALHNHYTQKSLSLSPGK (SEQ ID NO: 110) and an antibody light chain polypeptide comprising the amino acid sequence AIQMTQSPSSLSPSVGDRVTITCRASQGIRNDLGWYQQKPGKAPKLLIFAASSLQSGVPSRFSGSG SGTDFTLTISNLQPEDFATYYCLQNYNYPLTFGGGTKVEIKRTVAAPSVFIFPPSDEQLKSGTASV VCLLNNFYPREAKVQWKVDNALQSGNSQESVTEQDSKDSTYSLSSTLTLSKADYEKHKVYACE VTHQGLSSPVTKSFNRGEC (SEQ ID NO:83). For example, in some embodiments, a composition comprising an arm or multispecific binding molecule of the present disclosure comprises a mixture of species in which some species comprise an antibody heavy chain polypeptide comprising the amino acid sequence of SEQ ID NO: 109 and an antibody light chain polypeptide comprising the amino acid sequence of SEQ ID NO:83, while other species comprise an antibody heavy chain polypeptide comprising the amino acid sequence of SEQ ID NO: 110 and an antibody light chain polypeptide comprising the amino acid sequence of SEQ ID NO:83.

In some embodiments, the anti-Trop-2 antigen binding domain, anti-Trop-2 arm, or multispecific binding protein binds to human Trop-2. In some embodiments, the anti-Trop-2 antigen binding domain, anti-Trop-2 arm, or multispecific binding protein binds to human Trop-2 expressed on the surface of a cell, e.g., a cancer or tumor cell. In some embodiments, human Trop-2 comprises the amino acid sequence

(SEQ ID NO: 86)
MARGPGLAPPPLRLPLLLLVLAAVTGHTAAQDNCTCPTNKMTVCSPDGPG
GRCQCRALGSGMAVDCSTLTSKCLLLKARMSAPKNARTLVRPSEHALVDN
DGLYDPDCDPEGRFKARQCNQTSVCWCVNSVGVRRTDKGDLSLRCDELVR
THHILIDLRHRPTAGAFNHSDLDAELRRLFRERYRLHPKFVAAVHYEQPT
IQIELRQNTSQKAAGDVDIGDAAYYFERDIKGESLFQGRGGLDLRVRGEP
LQVERTLIYYLDEIPPKFSMKRLTAGLIAVIVVVVVALVAGMAVLVITNR
RKSGKYKKVEIKELGELRKEPSL.

### Multispecific Binding Molecule Formats and Other Features

In some embodiments, an antigen binding arm (e.g., an anti-Dectin-1 or anti-Trop-2 arm of the present disclosure) or multispecific binding molecule of the present disclosure comprises an Fc region. An antibody may be of any class or subclass, including IgG and subclasses thereof (IgG1, IgG2, IgG3, IgG4), IgM, IgE, IgA, and IgD. An immunoglobulin Fc region of the molecule that causes targeted phagocytosis may have important role in the process by engaging Fc receptors and inducing additional phagocytosis.

In some embodiments, the Fc region is a human IgG Fc region. In some embodiments, the Fc region is a human IgG1 or human IgG4 Fc region. In some embodiments, the Fc region is a human IgG1 Fc region comprising S239D and 1332E substitutions, according to EU numbering. In some embodiments, the Fc region is a human IgG1 Fc region comprising S239D, A330L, and I332E substitutions, according to EU numbering. In some embodiments, the Fc region is a human IgG1 Fc region comprising G236A, S239D, A330L, and 1332E substitutions, according to EU numbering. In some embodiments, the Fc region is a human IgG4 Fc region comprising an S228P substitution, according to EU numbering.

In some embodiments, an antigen binding arm (e.g., an anti-Dectin-1 or anti-Trop-2 arm of the present disclosure) or multispecific binding molecule of the present disclosure comprises an Fc region wherein a carbohydrate structure attached to the Fc region has reduced fucose or lacks fucose, e.g., at least one or two of the heavy chains of the multispecific binding molecule is non-fucosylated or comprise reduced fucosylation. In some embodiments, provided herein is a composition comprising a multispecific binding molecule of the present disclosure that comprises one or two Fc region(s) wherein a carbohydrate structure attached to the Fc region has reduced fucose or lacks fucose, e.g., at least one or two of the heavy chains of the multispecific binding molecule is/are non-fucosylated or comprise(s) reduced fucosylation. In some embodiments, less than 50% of the N-glycoside-linked carbohydrate chains in the composition contain a fucose residue. In some embodiments, substantially none of the N-glycoside-linked carbohydrate chains contain a fucose residue. In some embodiments, a multispecific binding molecule with reduced fucose or lacking fucose has improved ADCC function.

In some embodiments, multispecific (e.g., bispecific) binding molecules of the present disclosure further comprise one or more mutations on only one of the antibody arms to reduce binding affinity for Protein A. See, e.g., Ollier, R, et al. (2019) MAbs 11:1464-1478 and AU2018204314. In some embodiments, the multispecific (e.g., bispecific) binding molecule comprises two antibody light chains and two antibody heavy chains, wherein only one of the antibody heavy chains comprises amino acid substitutions H435R and Y436F, according to EU numbering.

In some embodiments, the multispecific (e.g., bispecific) binding molecules of the present disclosure further comprise one or more mutations to reduce effector function, e.g., to reduce or eliminate binding of the Fc region to an Fc receptor. In some embodiments, the multispecific (e.g., bispecific) binding molecule comprises two antibody Fc regions, wherein the antibody Fc regions comprise an amino acid substitution at one or more of positions 234, 235, and 237, according to EU numbering. In some embodiments, the multispecific (e.g., bispecific) binding molecule comprises two antibody Fc regions, wherein the antibody Fc regions comprise L234A, L235E, and G237A substitutions, according to EU numbering.

In other embodiments, a multispecific binding molecule of the present disclosure (e.g., an IgG1 multispecific binding molecule) or composition comprising a multispecific binding molecule of the present disclosure (e.g., an IgG1 multispecific binding molecule) comprises wild-type glycosylation of one or two Fc region(s). In some embodiments, provided herein are fucosylated multispecific binding molecules of the present disclosure (e.g., an IgG1 multispecific binding molecule) or compositions comprising a fucosylated multispecific binding molecule of the present disclosure (e.g., an IgG1 multispecific binding molecule).

Fucosylation or fucosylated antibodies can refer to the presence of fucose residues within the oligosaccharides attached to the peptide backbone of an antibody. Specifically, a fucosylated antibody comprises α (1,6)-linked fucose at the innermost N-acetylglucosamine (GlcNAc) residue in one or both of the N-linked oligosaccharides attached to the antibody Fc region, e.g., at position Asn 297 of the human IgG1 Fc region (EU numbering of Fc region residues). Asn297 may also be located about +3 amino acids upstream or downstream of position 297, i.e., between positions 294 and 300, due to minor sequence variations in immunoglobulins. Non-fucosylated or fucose-deficient antibodies have reduced fucose relative to the amount of fucose on the same antibody produced in a cell line Antibody fucosylation can be measured, e.g., in an N-glycosidase F treated antibody composition assessed by matrix-assisted laser desorption-ionization time-of-flight mass spectrometry (MALDI TOF MS).

In some embodiments, the Fe region comprises one or more mutations that reduce or eliminate fucosylation, e.g., a substitution at Asn 297 of the human IgG1 Fc region (EU numbering of Fc region residues). Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87:614 (2004).

Various technologies for assembling two arms of a multispecific binding molecule of the present disclosure are known in the art and contemplated for use herein.

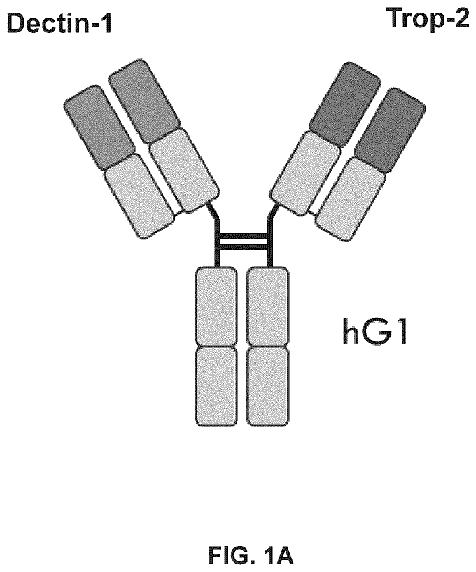

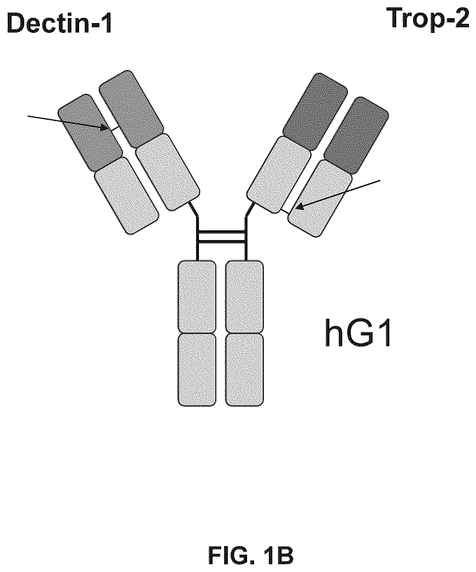

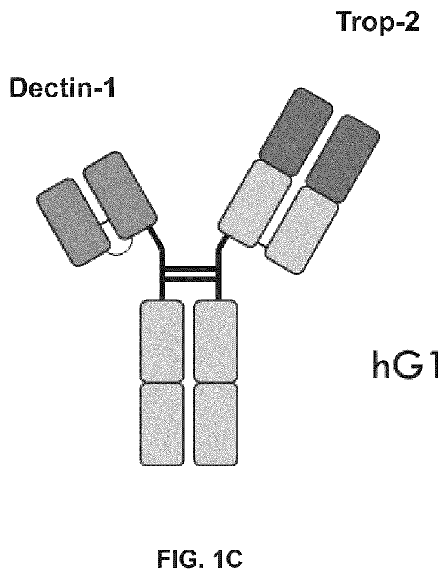

In some embodiments, the multispecific (e.g., bispecific) binding molecule of the present disclosure comprises a first antibody arm comprising an antibody heavy chain that comprises a first VH domain in association with an antibody light chain that comprises a first VL domain, and a first Fc region connected to the first VH domain, wherein the first VH and VL domains form a first antigen-binding domain that binds to human Dectin-1, and a second antibody arm comprising an antibody heavy chain that comprises a second VH domain in association with an antibody light chain that comprises a second VL domain, and a second Fc region connected to the second VH domain, wherein the second VH and VL domains form a second antigen-binding domain that binds to human Trop-2. That is, the first and second antibody arms can be of conventional antibody format (see, e.g., FIGS. 1A & 1B).

One well-established approach for making bispecific antibodies is the “knobs-into-holes” or “protuberance-into-cavity” approach. See e.g., U.S. Pat. No. 5,731,168. Two immunoglobulin polypeptides (e.g., heavy chain polypeptides) each comprise an interface; an interface of one immunoglobulin polypeptide interacts with a corresponding or cognate interface on the other immunoglobulin polypeptide, thereby allowing the two immunoglobulin polypeptides to associate. In some embodiments, interfaces may be engineered such that a “knob” or “protuberance” located in the interface of one immunoglobulin polypeptide corresponds with a cognate “hole” or “cavity” located in the interface of the other immunoglobulin polypeptide. In some embodiments, a knob may be constructed by replacing a small amino acid side chain with a larger side chain. In some embodiments, a hole may be constructed by replacing a large amino acid side chain with a smaller side chain. Knobs or holes may exist in the original interface, or they may be introduced synthetically. Polynucleotides encoding modified immunoglobulin polypeptides with one or more corresponding knob- or hole-forming mutations may be expressed and purified using standard recombinant techniques and cell systems known in the art. See, e.g., U.S. Pat. Nos. 5,731,168; 5,807,706; 5,821,333; 7,642,228; 7,695,936; 8,216,805; 8,679,785; 8,844,834; U.S. Pub. No. 2013/0089553; Spiess et al., Nature Biotechnology 31:753-758, 2013; and Ridgway and Carter (1996) Protein Eng. 9:617-621. Modified immunoglobulin polypeptides may be produced using prokaryotic host cells, such as E. coli, or eukaryotic host cells, such as mammalian cells (e.g., CHO cells) or yeast cells. Corresponding knob- and hole-bearing immunoglobulin polypeptides may be expressed in host cells in co-culture and purified together as a heteromultimer, or they may be expressed in single cultures, separately purified, and assembled in vitro. Exemplary cognate knob and hole mutations are provided below (numbering according to EU index). EU numbering as used herein is known in the art; see, e.g., IMGT resources at www.imgt.org/IMGTScientificChart/Numbering/Hu_IGHGnber.html and www.imgt.org/IMGTScientificChart/Numbering/Hu_IGKCnber.html.

| | | | | | | | | |
|---|---|---|---|---|---|---|---|---|
| Fc region 1 | Y407T | Y407A | F405A | T394S | T366S | T394W | T394S | T366W |
| | | | | | L368A | Y407T | Y407A | T394S |
| | | | | | Y407V | | | |
| Fc region 2 | T366Y | T366W | T394W | F405W | T366W | T366Y | T366W | F405W |
| | | | | | | F405A | F405W | Y407A |

In some embodiments, provided herein is a multispecific (e.g., bispecific) binding molecule that comprises a first antibody arm comprising a first antibody heavy chain that comprises a first VH domain in association with a first antibody light chain that comprises a first VL domain and a first Fc region connected to the first VH domain, wherein the first VH and VL domains form an antigen binding domain that binds to human Dectin-1, and a second antibody arm comprising a second antibody heavy chain that comprises a second VH domain in association with a second antibody light chain that comprises a second VL domain and a second Fc region connected to the second VH domain, wherein the second VH and VL domains form an antigen binding domain that binds to human Trop-2. In some embodiments, the first Fc region comprises one or more knob-forming mutations, and the second Fc region comprises one or more cognate hole-forming mutations, or the second Fc region comprises one or more knob-forming mutations, and the first Fc region comprises one or more cognate hole-forming mutations. Non-limiting sets of knob- and hole-forming mutations include T366W for knob-forming and T366S, L368A, and Y407V substitutions for hole forming, according to EU numbering.

In some embodiments, the multispecific (e.g., bispecific) binding molecules of the present disclosure comprise two antibody heavy chains and two antibody light chains, wherein the VH domain of the first antibody heavy chain forms an antigen binding domain with the VL domain of the first antibody light chain, wherein the VH domain of the second antibody heavy chain forms an antigen binding domain with the VL domain of the second antibody light chain, wherein the first antibody heavy chain comprises F126C, C220V, and T366W substitutions, wherein the first antibody light chain comprises S121C and C214V substitutions, and wherein the second antibody heavy chain comprises T366S, L368A, Y407V, H435R, and Y436F substitutions, according to EU numbering. In some embodiments, the first and second antibody heavy chains further comprise L234A, L235E, and G237A substitutions, according to EU numbering. In some embodiments, the first and second antibody heavy chains comprise human IgG1 Fc domains.

In some embodiments, multispecific (e.g., bispecific) antibodies further comprise one or more mutations on only one of the antibody arms to improve heavy chain/light chain pairing. For example, amino acid substitutions can be used to replace a native disulfide bond in the CH1-CL interface of one antibody arm with an engineered disulfide bond (see, e.g., FIG. 1B). See, e.g., Mazor. Y, et al. (2015) MAbs 7:377-389 and EP3452089A2. In some embodiments, the multispecific or bispecific antibody comprises two antibody light chains and two antibody heavy chains, wherein only one of the antibody heavy chains comprises amino acid substitutions F126C and C220V, and only the corresponding or cognate light chain comprises amino acid substitutions S121C and C214V, according to EU numbering.

In some embodiments, the antibody heavy chain polypeptide of an arm of a multispecific (e.g., bispecific) binding molecule of the present disclosure has a C→S amino acid substitution at position 5, according to IMGT hinge numbering; position 220, according to EU index; or position 233, according to Kabat numbering. Descriptions of the IMGT hinge, EU index, and Kabat numbering schemes are known in the art (see, e.g., imgt.org/IMGTScientificChart/Numbering/Hu_IGHGnber.html), and therefore the precise location of this residue can be determined by the skilled person using knowledge common in the art. In some embodiments, the antibody light chain polypeptide of the corresponding arm of the multispecific (e.g., bispecific) binding molecule has a C→S at the terminal residue of the light chain constant domain (e.g., CK domain). In some embodiments, the VH domain comprises the amino acid sequence of SEQ ID NO: 30; the VL domain comprises the amino acid sequence of SEQ ID NO: 31; the antibody heavy chain of the antibody arm has a C→S amino acid substitution at position 5, according to IMGT hinge numbering; position 220, according to EU index; or position 233, according to Kabat numbering; and the antibody light chain of the antibody arm has a C→S at the terminal residue of the light chain constant domain (e.g., CK domain). In some embodiments, the VH domain of the anti-Dectin-1 arm comprises the amino acid sequence of SEQ ID NO: 30, and the VL domain of the anti-Dectin-1 arm comprises the amino acid sequence of SEQ ID NO:31. In some embodiments, the VH and VL domains of the anti-Dectin-1 arm are part of a scFv that comprises the amino acid sequence of SEQ ID NO:34.

In some embodiments, the multispecific (e.g., bispecific) binding molecule of the present disclosure comprises a first antibody arm comprising a single chain variable fragment (scFv) comprising VH and VL domains of the present disclosure that bind to human Dectin-1 and a first Fc region, and a second antibody arm comprising an antibody heavy chain that comprises a VH domain in association with an antibody light chain that comprises a VL domain, and a second Fc region connected to the VH domain, wherein the VH and VL domains of the second antibody arm form an antigen-binding domain that binds to Trop-2. That is, the first arm can be of scFv format (e.g., scFv-Fc fusion), and the second antibody arm can be of conventional antibody format (see, e.g., FIG. 1C).

In some embodiments, provided herein is a multispecific (e.g., bispecific) binding molecule that comprises a first antibody arm comprising a single chain variable fragment (scFv) comprising VH and VL domains of the present disclosure that bind to human Dectin-1 and a first Fc region, and a second antibody arm comprising an antibody heavy chain that comprises a VH domain in association with an antibody light chain that comprises a VL domain and a second Fc region connected to the VH domain, wherein the VH and VL domains of the second antibody arm form an antigen binding domain that binds to human Trop-2. In some embodiments, the first Fc region comprises one or more knob-forming mutations, and the second Fc region comprises one or more cognate hole-forming mutations, or the second Fc region comprises one or more knob-forming mutations, and the first Fc region comprises one or more cognate hole-forming mutations. In some embodiments, the scFv comprises a first linker of the present disclosure between the VH and VL domains and a second linker of the present disclosure between the VL domain and the first Fc region. Non-limiting sets of knob- and hole-forming mutations include T366W for knob-forming and T366S. L368A, and Y407V substitutions for hole forming, according to EU numbering.

According to a different approach, antibody variable domains with the desired binding specificities (antibody-antigen combining sites) are fused to immunoglobulin constant domain sequences.

Multispecific (e.g., bispecific) binding molecules also include cross-linked or “heteroconjugate” antibodies. Techniques for generating bispecific antibodies from antibody fragments have also been described in the literature. For example, multispecific (e.g., bispecific) binding molecules can be prepared using chemical linkage. In some embodiments, a multispecific (e.g., bispecific) binding molecule comprises a first IgG antibody comprising the first antigen binding domain covalently linked to a second IgG antibody comprising the second antigen binding domain.

In some embodiments, the first arm is coupled to avidin, streptavidin, neutravidin, or a biotin-binding derivative thereof, and the second arm is coupled to biotin or an avidin-binding derivative thereof. In some embodiments, the second arm is coupled to avidin, streptavidin, neutravidin, or a biotin-binding derivative thereof, and the first arm is coupled to biotin or an avidin-binding derivative thereof. In some embodiments, the first arm is bound to the second arm via an interaction between the avidin, streptavidin, neutravidin, or biotin-binding derivative thereof and the biotin or avidin-binding derivative thereof. Exemplary avidins, streptavidins, neutravidins, or biotin-binding derivatives thereof are known in the art. In some embodiments, the streptavidin is monomeric streptavidin (mSA). Exemplary biotins or avidin-binding derivatives thereof are known in the art. In some embodiments, an antigen binding arm thereof of the present disclosure is biotinylated.

In some embodiments, one or both of the first and second arms comprise(s) a tag, e.g., for affinity purification. In some embodiments, the tag is a polyhistidine tag.

In some embodiments, one or both of the first and second arms are Fab, Fab′, F(ab′)₂, Fv, Fab′-SH, F(ab′)2, single chain antibodies, nanobodies, or scFv fragments. In some embodiments, one or both of the first and second arms further comprise an Fc domain. In some embodiments, the first arm is a Fab fragment, and the second arm is a full-length antibody. In some embodiments, the first and the second arms are both full-length half antibodies.

In some embodiments, a multispecific (e.g., bispecific) binding molecule of the present disclosure comprises a first arm (e.g., an anti-Dectin-1 arm) that comprises a first antibody heavy chain polypeptide and a first antibody light chain polypeptide and a second arm (e.g., an anti-Trop-2 arm) that comprises a second antibody heavy chain polypeptide and a second antibody light chain polypeptide. In some embodiments, the first antibody heavy chain polypeptide comprises the amino acid sequence of SEQ ID NO:87 or 88, the first antibody light chain polypeptide comprises the amino acid sequence of SEQ ID NO:89, the second antibody heavy chain polypeptide comprises the amino acid sequence of SEQ ID NO: 71 or 72, and the second antibody light chain polypeptide comprises the amino acid sequence of SEQ ID NO:73. In some embodiments, the first antibody heavy chain polypeptide comprises the amino acid sequence of SEQ ID NO:87 or 88, the first antibody light chain polypeptide comprises the amino acid sequence of SEQ ID NO:89, the second antibody heavy chain polypeptide comprises the amino acid sequence of SEQ ID NO:76 or 77, and the second antibody light chain polypeptide comprises the amino acid sequence of SEQ ID NO:78. In some embodiments, the first antibody heavy chain polypeptide comprises the amino acid sequence of SEQ ID NO:87 or 88, the first antibody light chain polypeptide comprises the amino acid sequence of SEQ ID NO:89, the second antibody heavy chain polypeptide comprises the amino acid sequence of SEQ ID NO:81 or 82, and the second antibody light chain polypeptide comprises the amino acid sequence of SEQ ID NO:83. In some embodiments, the first antibody heavy chain polypeptide comprises the amino acid sequence of SEQ ID NO:28 or 90, the first antibody light chain polypeptide comprises the amino acid sequence of SEQ ID NO:29, the second antibody heavy chain polypeptide comprises the amino acid sequence of SEQ ID NO:71 or 72, and the second antibody light chain polypeptide comprises the amino acid sequence of SEQ ID NO:73. In some embodiments, the first antibody heavy chain polypeptide comprises the amino acid sequence of SEQ ID NO: 28 or 90, the first antibody light chain polypeptide comprises the amino acid sequence of SEQ ID NO: 29, the second antibody heavy chain polypeptide comprises the amino acid sequence of SEQ ID NO: 76 or 77, and the second antibody light chain polypeptide comprises the amino acid sequence of SEQ ID NO: 78. In some embodiments, the first antibody heavy chain polypeptide comprises the amino acid sequence of SEQ ID NO:28 or 90, the first antibody light chain polypeptide comprises the amino acid sequence of SEQ ID NO:29, the second antibody heavy chain polypeptide comprises the amino acid sequence of SEQ ID NO:81 or 82, and the second antibody light chain polypeptide comprises the amino acid sequence of SEQ ID NO:83. In some embodiments, the first antibody heavy chain polypeptide comprises the amino acid sequence of SEQ ID NO:32 or 91, the first antibody light chain polypeptide comprises the amino acid sequence of SEQ ID NO:33, the second antibody heavy chain polypeptide comprises the amino acid sequence of SEQ ID NO:71 or 72, and the second antibody light chain polypeptide comprises the amino acid sequence of SEQ ID NO:73. In some embodiments, the first antibody heavy chain polypeptide comprises the amino acid sequence of SEQ ID NO:32 or 91, the first antibody light chain polypeptide comprises the amino acid sequence of SEQ ID NO:33, the second antibody heavy chain polypeptide comprises the amino acid sequence of SEQ ID NO:76 or 77, and the second antibody light chain polypeptide comprises the amino acid sequence of SEQ ID NO:78. In some embodiments, the first antibody heavy chain polypeptide comprises the amino acid sequence of SEQ ID NO: 32 or 91, the first antibody light chain polypeptide comprises the amino acid sequence of SEQ ID NO: 33, the second antibody heavy chain polypeptide comprises the amino acid sequence of SEQ ID NO: 81 or 82, and the second antibody light chain polypeptide comprises the amino acid sequence of SEQ ID NO: 83. In some embodiments, the first antibody heavy chain polypeptide comprises the amino acid sequence of SEQ ID NO: 111 or 112, the first antibody light chain polypeptide comprises the amino acid sequence of SEQ ID NO:89, the second antibody heavy chain polypeptide comprises the amino acid sequence of SEQ ID NO: 105 or 106, and the second antibody light chain polypeptide comprises the amino acid sequence of SEQ ID NO:73. In some embodiments, the first antibody heavy chain polypeptide comprises the amino acid sequence of SEQ ID NO: 111 or 112, the first antibody light chain polypeptide comprises the amino acid sequence of SEQ ID NO:89, the second antibody heavy chain polypeptide comprises the amino acid sequence of SEQ ID NO: 107 or 108, and the second antibody light chain polypeptide comprises the amino acid sequence of SEQ ID NO:78. In some embodiments, the first antibody heavy chain polypeptide comprises the amino acid sequence of SEQ ID NO: 111 or 112, the first antibody light chain polypeptide comprises the amino acid sequence of SEQ ID NO:89, the second antibody heavy chain polypeptide comprises the amino acid sequence of SEQ ID NO: 109 or 110, and the second antibody light chain polypeptide comprises the amino acid sequence of SEQ ID NO:83. In some embodiments, the first antibody heavy chain polypeptide comprises the amino acid sequence of SEQ ID NO: 99 or 100, the first antibody light chain polypeptide comprises the amino acid sequence of SEQ ID NO: 29, the second antibody heavy chain polypeptide comprises the amino acid sequence of SEQ ID NO: 105 or 106, and the second antibody light chain polypeptide comprises the amino acid sequence of SEQ ID NO:73. In some embodiments, the first antibody heavy chain polypeptide comprises the amino acid sequence of SEQ ID NO:99 or 100, the first antibody light chain polypeptide comprises the amino acid sequence of SEQ ID NO:29, the second antibody heavy chain polypeptide comprises the amino acid sequence of SEQ ID NO: 107 or 108, and the second antibody light chain polypeptide comprises the amino acid sequence of SEQ ID NO:78. In some embodiments, the first antibody heavy chain polypeptide comprises the amino acid sequence of SEQ ID NO:99 or 100, the first antibody light chain polypeptide comprises the amino acid sequence of SEQ ID NO:29, the second antibody heavy chain polypeptide comprises the amino acid sequence of SEQ ID NO: 109 or 110, and the second antibody light chain polypeptide comprises the amino acid sequence of SEQ ID NO:83. In some embodiments, the first antibody heavy chain polypeptide comprises the amino acid sequence of SEQ ID NO: 101 or 102, the first antibody light chain polypeptide comprises the amino acid sequence of SEQ ID NO:33, the second antibody heavy chain polypeptide comprises the amino acid sequence of SEQ ID NO: 105 or 106, and the second antibody light chain polypeptide comprises the amino acid sequence of SEQ ID NO: 73. In some embodiments, the first antibody heavy chain polypeptide comprises the amino acid sequence of SEQ ID NO: 101 or 102, the first antibody light chain polypeptide comprises the amino acid sequence of SEQ ID NO: 33, the second antibody heavy chain polypeptide comprises the amino acid sequence of SEQ ID NO: 107 or 108, and the second antibody light chain polypeptide comprises the amino acid sequence of SEQ ID NO:78. In some embodiments, the first antibody heavy chain polypeptide comprises the amino acid sequence of SEQ ID NO: 101 or 102, the first antibody light chain polypeptide comprises the amino acid sequence of SEQ ID NO:33, the second antibody heavy chain polypeptide comprises the amino acid sequence of SEQ ID NO: 109 or 110, and the second antibody light chain polypeptide comprises the amino acid sequence of SEQ ID NO:83.

In some embodiments, a multispecific (e.g., bispecific) binding molecule of the present disclosure comprises a first arm (e.g., an anti-Dectin-1 arm) that comprises a single chain variable fragment (scFv) linked to an Fc region and a second arm (e.g., an anti-Trop-2 arm) that comprises an antibody heavy chain polypeptide and an antibody light chain polypeptide. In some embodiments, the first arm comprises the amino acid sequence of SEQ ID NO:35 or 36, the antibody heavy chain polypeptide comprises the amino acid sequence of SEQ ID NO:71 or 72, and the antibody light chain polypeptide comprises the amino acid sequence of SEQ ID NO: 73. In some embodiments, the first arm comprises the amino acid sequence of SEQ ID NO:35 or 36, the antibody heavy chain polypeptide comprises the amino acid sequence of SEQ ID NO:76 or 77, and the antibody light chain polypeptide comprises the amino acid sequence of SEQ ID NO:78. In some embodiments, the first arm comprises the amino acid sequence of SEQ ID NO:35 or 36, the antibody heavy chain polypeptide comprises the amino acid sequence of SEQ ID NO:81 or 82, and the antibody light chain polypeptide comprises the amino acid sequence of SEQ ID NO:83. In some embodiments, the first arm comprises the amino acid sequence of SEQ ID NO: 103 or 104, the antibody heavy chain polypeptide comprises the amino acid sequence of SEQ ID NO: 105 or 106, and the antibody light chain polypeptide comprises the amino acid sequence of SEQ ID NO:73. In some embodiments, the first arm comprises the amino acid sequence of SEQ ID NO: 103 or 104, the antibody heavy chain polypeptide comprises the amino acid sequence of SEQ ID NO: 107 or 108, and the antibody light chain polypeptide comprises the amino acid sequence of SEQ ID NO: 78. In some embodiments, the first arm comprises the amino acid sequence of SEQ ID NO: 103 or 104, the antibody heavy chain polypeptide comprises the amino acid sequence of SEQ ID NO: 109 or 110, and the antibody light chain polypeptide comprises the amino acid sequence of SEQ ID NO:83.

In some embodiments, provided herein is a polynucleotide encoding the multispecific binding molecule of any one of the above embodiments. In some embodiments, provided herein is a vector (e.g., an expression vector) comprising the polynucleotide of any one of the above embodiments. In some embodiments, each arm of a multispecific (e.g., bispecific) binding molecule of the present disclosure is encoded by a separate polynucleotide or vector. In some embodiments, a single polynucleotide or vector encodes both arms of a multispecific (e.g., bispecific) binding molecule of the present disclosure.

In some embodiments, provided herein is a host cell (e.g., an isolated host cell or cell line) comprising the polynucleotide or vector of any one of the above embodiments. In some embodiments, the host cell is a eukaryotic host cell. In some embodiments, the host cell is an insect or mammalian host cell. Any of these may find use in the methods of production and/or treatment disclosed herein. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather. Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; CHOK1 cells, CHOK1SV cells or derivatives and a human hepatoma line (Hep G2).

In some embodiments, an afucosylated or non-fucosylated multispecific binding molecule is produced in a cell line with a genetic modification that results in an afucosylated or non-fucosylated multispecific binding molecule. Examples of cell lines producing afucosylated products include Lec 13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87:614 (2004)), cells overexpressing β1,4-N-acetylglucosaminyltransferase III (GnT-III) and Golgi μ-mannosidase II (ManII), and cells with a knockout in the mannosyl-glycoprotein 2-beta-N-acetylglucosaminyltranferase (MGAT1; see Byrne, G, et al. (2018) PLoS Biol. 16:02005817).

In some embodiments, an afucosylated or non-fucosylated multispecific binding molecule is produced in a cell line treated with an inhibitor of glycoprocessing enzyme(s), such as kifunensine, which is an inhibitor of mannosidase I (see, e.g., Elbein, A. D, et al. (1990) J. Biol. Chem. 265:15599-15605). For example, cells can be centrifuged and resuspended in growth medium comprising kifunensine (e.g., at 250 μg/mL), then cultured and used for production.

In some embodiments, provided herein is a method of producing a multispecific binding molecule, comprising culturing the host cell of any one of the above embodiments under conditions suitable for production of the multispecific binding molecule. In some embodiments, the method further comprises recovering the multispecific binding molecule. The multispecific binding molecules may be produced using standard recombinant techniques, as described herein, and/or as exemplified infra.

Antibodies and antibody fragments may be produced using recombinant methods. For example, nucleic acid encoding the antibody/fragment can be isolated and inserted into a replicable vector for further cloning or for expression. DNA encoding the antibody/fragment may be readily isolated and sequenced using conventional procedures (e.g., via oligonucleotide probes capable of binding specifically to genes encoding the heavy and light chains of the antibody/fragment). Many vectors are known in the art; vector components generally include, but are not limited to, one or more of the following: a signal sequence, an origin of replication, one or more marker genes, an enhancer element, a promoter, and a transcription termination sequence. Suitable host cells for cloning or expressing the DNA in the vectors herein are the prokaryote, yeast, or higher eukaryote cells. When using recombinant techniques, the antibody/fragment can be produced intracellularly, in the periplasmic space, or directly secreted into the medium. If the antibody/fragment is produced intracellularly, the particulate debris, either host cells or lysed fragments, are removed, for example, by centrifugation or ultrafiltration. Where the antibody/fragment is secreted into the medium, supernatants from such expression systems are generally first concentrated using a commercially available protein concentration filter.

### II. Methods of Use

In certain aspects, the present disclosure provides methods of treating cancer, comprising administering an effective amount of a multispecific (e.g., bispecific) binding molecule or composition of the present disclosure to an individual in need thereof. In some embodiments, the individual is a human. In some embodiments, the individual has or has been diagnosed with cancer. Any of the multispecific binding molecules of the present disclosure (e.g., as described supra in section I) may find use in the methods of treatment and uses disclosed herein, as well as the compositions (e.g., pharmaceutical compositions) related thereto.

In some embodiments, the cancer is a solid cancer. In some embodiments, the cancer is wherein the cancer is breast cancer, prostate cancer, urothelial cancer, cervical cancer, bladder cancer, pancreatic cancer, thyroid cancer, head and neck cancer, esophageal cancer, endometrial cancer, gastric cancer, liver cancer, colorectal cancer, skin cancer (e.g., melanoma), lung cancer, ovarian cancer, or uterine cancer. In some embodiments, cells of the cancer express Trop-2. Exemplary cancer types demonstrated herein to express Trop-2 and contemplated for treatment with the methods of the present disclosure include, without limitation, cervical squamous cell carcinoma, bladder urothelial carcinoma, head and neck squamous cell carcinoma (HNSCC), esophageal squamous cell carcinoma, endocervical adenocarcinoma, lung squamous cell carcinoma, prostate adenocarcinoma, papillary thyroid cancer, mucinous carcinoma, endometrioid carcinoma, pancreatic adenocarcinoma, breast invasive lobular carcinoma, lung adenocarcinoma, uterine endometrioid carcinoma, serous ovarian cancer, breast invasive carcinoma (NOS), breast invasive ductal carcinoma, uterine serous carcinoma, metaplastic breast cancer, uterine mixed endometrial carcinoma, breast invasive mixed mucinous carcinoma, papillary renal cell carcinoma, esophageal adenocarcinoma, stomach adenocarcinoma, invasive breast carcinoma, intrahepatic cholangiocarcinoma, cholangiocarcinoma, tubular stomach adenocarcinoma, intestinal type stomach adenocarcinoma, mucinous stomach adenocarcinoma, papillary stomach adenocarcinoma, diffuse type stomach adenocarcinoma, teratoma, signet ring cell carcinoma of the stomach, mucinous adenocarcinoma of the colon/rectum, uterine carcinosarcoma, rectal adenocarcinoma, thymoma, and perihilar cholangiocarcinoma. In some embodiments, a sample obtained from the cancer of the individual is positive for Trop-2 expression. In some embodiments, the methods further comprise obtaining a sample from the cancer of the individual, wherein the sample comprises cancer cells (e.g., that express Trop-2).

In some embodiments, the cancer is responsive to single-agent immunotherapy (e.g., single-agent treatment with immune checkpoint inhibitor), e.g., prior to administration of the multispecific (e.g., bispecific) binding molecule. In some embodiments, the cancer is not responsive to single-agent immunotherapy (e.g., single-agent treatment with immune checkpoint inhibitor), e.g., prior to administration of the multispecific (e.g., bispecific) binding molecule. Advantageously, as demonstrated herein, the multispecific binding molecules of the present disclosure have anti-tumor activity against tumor models with varying tumor microenvironments, including models of both “hot” (e.g., characterized by immune cell infiltration and/or responsiveness to single-agent treatment with an immunotherapy such as an immune checkpoint inhibitor) and “cold” (e.g., characterized by lack of immune cell infiltration and/or reduced or minimal responsiveness to single-agent treatment with an immunotherapy such as an immune checkpoint inhibitor) tumors.

### III. Compositions and Kits

In certain aspects, the present disclosure provides pharmaceutical compositions comprising the multispecific (e.g., bispecific) binding molecule of any one of the embodiments disclosed herein and a pharmaceutically acceptable carrier.

Pharmaceutical compositions and formulations as described herein can be prepared by mixing the active ingredients (such as a multispecific binding molecule) having the desired degree of purity with one or more optional pharmaceutically acceptable carriers (Remington's Pharmaceutical Sciences 16th edition. Osol. A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Certain aspects of the present disclosure relate to kits or articles of manufacture comprising any of the multispecific binding molecules disclosed herein. In some embodiments, the article of manufacture comprises a container and a label or package insert on or associated with the container. In some embodiments, the kit or article of manufacture further comprises instructions for using the multispecific binding molecule according to any of the methods disclosed herein, e.g., for treating cancer.

Suitable containers include, for example, bottles, vials, syringes, etc. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). At least one active agent in the composition is an antibody or multispecific binding molecule as described herein. The label or package insert indicates that the composition is used for treating the particular condition. The label or package insert will further comprise instructions for administering the multispecific binding molecule composition to the subject. Articles of manufacture and kits comprising combinatorial therapies described herein are also contemplated.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Characterization of Anti-Dectin-1 x Trop-2 Bispecific Antibody Binding

This example characterizes the binding of anti-Dectin-1 x anti-Trop-2 bispecific antibodies to their targets.

### Materials and Methods

### Bispecific Antibodies

For the Fab format shown in FIG. 1A, the anti-Dectin-1 binding arm comprised variable domains with the amino acid sequences of SEQ ID NO:24 for the VH domain and SEQ ID NO:25 for the VL domain. For the scFv format shown in FIG. 1C, the anti-Dectin-1 binding arm comprised a scFv with the amino acid sequence of SEQ ID NO:34. For the Fab format shown in FIG. 1B with engineered disulfides in the anti-Dectin-1 arm, the anti-Dectin-1 binding arm comprised variable domains with the amino acid sequences of SEQ ID NO:30 for the VH domain and SEQ ID NO:31 for the VL domain.

For the anti-Trop-2 binding arm in all formats, variable domains had the amino acid sequences of SEQ ID NO:69 for the VH domain and SEQ ID NO:70 for the VL domain for Clone A, the amino acid sequences of SEQ ID NO: 74 for the VH domain and SEQ ID NO: 75 for the VL domain for Clone B, and the amino acid sequences of SEQ ID NO: 79 for the VH domain and SEQ ID NO:80 for the VL domain for Clone C, respectively. All bispecific antibodies used a human IgG1 Fc region.

### Flow Cytometry

Human cancer cell lines and CHO cells expressing human Trop-2 (CHO-K1-hTrop2 cells) were incubated with the anti-Dectin-1 x anti-Trop-2 bispecific antibodies, Trop-2 clones 15, 45, 76, 77, 63, 64, 65 and 123, or the hIgG1 isotype, followed by a fluorescent anti-human secondary antibody. The primary antibodies were used in an 8-point serial dose titration starting at 300 or 100 nM and diluted 1 to 3.

### Bio-Layer Interferometry (BLI)

Monovalent binding affinity was measured by biosensor (BLI). Human Trop-2 ECD protein was captured on biosensor via anti-HIS Tag. On and off rate binding was measured against bispecific antibodies with 1 arm binding to hTrop2 at varying concentrations. On rate and off rate measurements were fitted to generated K_{D} values.

### SEAP Secretion

HEK-Blue hDectin-1a cells engineered to express Dectin-1 isoform A and genes involved in the Dectin-1/NF-κB/SEAP signaling pathway (thus expressing a secreted alkaline phosphatase (SEAP) in response to stimulation by Dectin-1 ligands) were used. Human cancer cell lines expressing Trop-2 were treated with a serial dilution of the anti-Dectin-1 x anti-Trop-2 bispecific antibodies in the presence of the Dectin-1 expressing reporter HEK cell line. After 24 hours of treatment, supernatant was collected to test the Dectin-1 induced NF-κB driven SEAP activity.

### Results

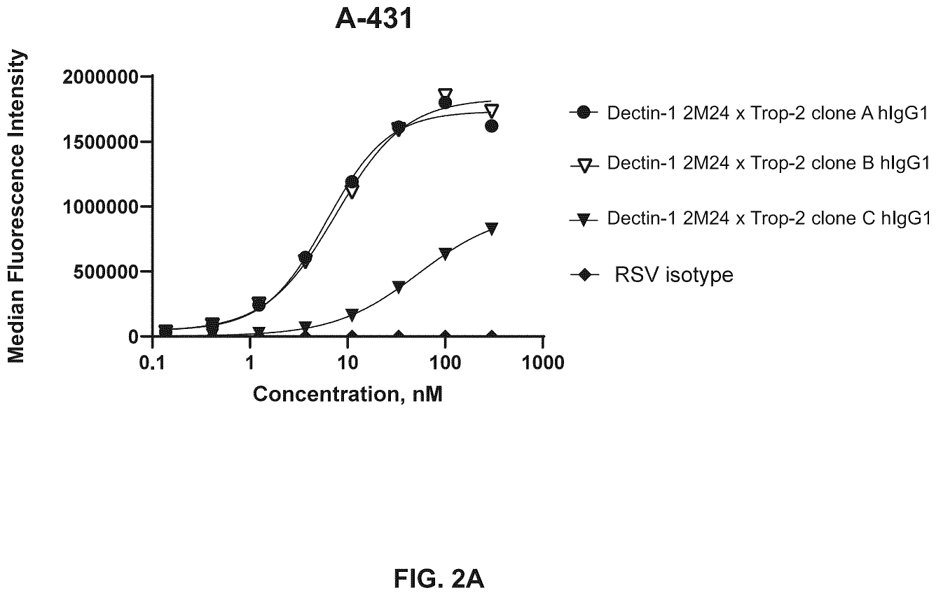

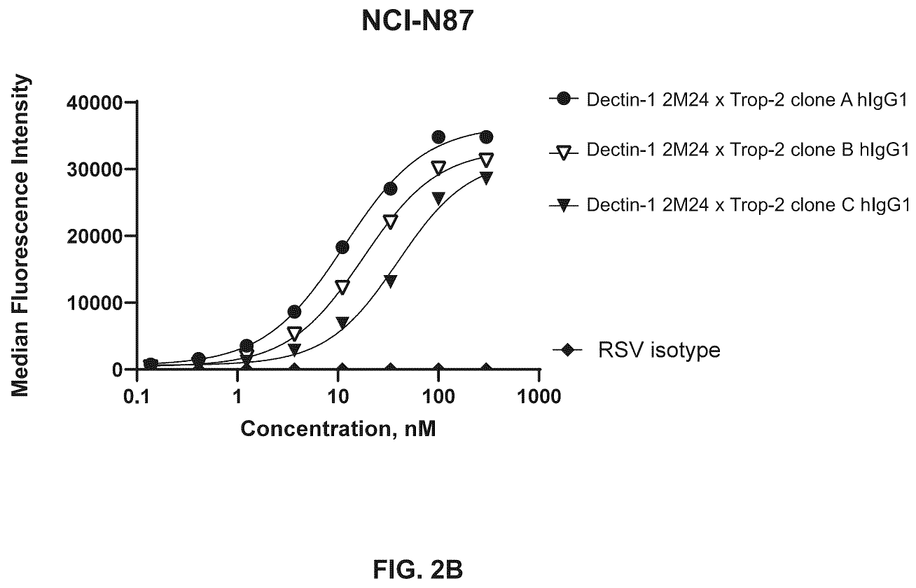

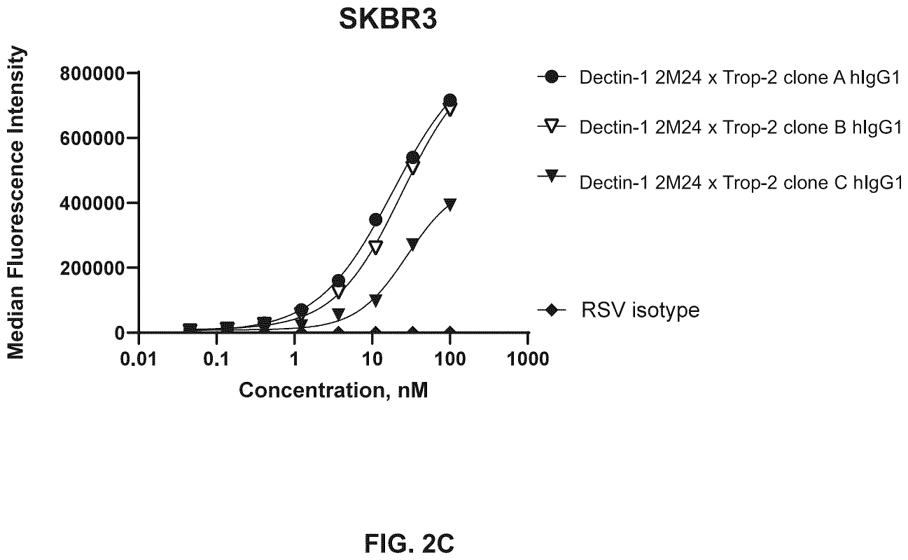

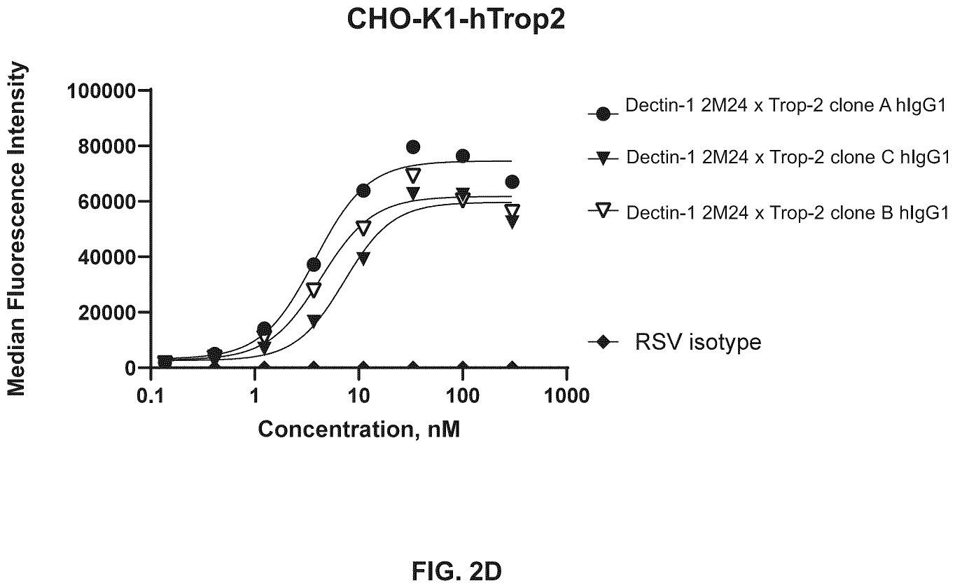

All three anti-Dectin-1 x anti-Trop-2 bispecific antibodies demonstrated high-affinity binding to cells expressing human Trop-2, including human cancer cell lines and CHO cells engineered to express human Trop-2 (FIGS. 2A-2D). EC50 binding values of each antibody to each target are shown in Table 1. Without wishing to be bound to theory, it is thought that anti-Dectin-1 x anti-Trop-2 bispecific antibodies can efficiently couple myeloid cells to Trop-2-expressing cancer cells, promoting Dectin-1-mediated immune stimulation, tumor cell phagocytosis, and antigen presentation. Advantageously, anti-Trop-2 clones B and C are from humanized mice (as opposed to being humanized antibodies like clone A) and therefore may be more human-like and pose less immunogenicity risk.

TABLE 1
Binding of anti-Dectin-1 × anti-Trop-2 bispecific
antibodies to cell lines tested (EC50, nM).

| | | | | CHO-K1- |
|---|---|---|---|---|
| Bispecific Antibody | A-431 | NCI-N87 | SKBR3 | hTrop2 |
| 2M24 × Trop-2 clone A hG1 | 6.12 | 11.68 | 18.47 | 3.78 |
| 2M24 × Trop-2 clone C hG1 | 53.23 | 39.46 | 28.04 | 7.25 |
| 2M24 × Trop-2 clone B hG1 | 7.563 | 17.85 | 24.22 | 4.32 |

Monovalent binding affinity of each anti-Dectin-1 x anti-Trop-2 bispecific antibody to the human Trop-2 ECD was also measured by biosensor (BLI). Measurements are provided in Table 2.

TABLE 2
Characterization of anti-Dectin-1 × anti-Trop-2
bispecific antibody binding to hTrop-2 ECD by BLI.

| Clone | K_{off} (1/s) | Kₒₙ (1/Ms) | K_{D} (M) | Bin |
|---|---|---|---|---|
| Clone A | 8.91E−04 | 2.17E+05 | 4.12E−09 | A |
| Clone B | 4.75E−04 | 4.97E+04 | 9.55E−09 | A |
| Clone C | 1.81E−03 | 1.40E+05 | 1.29E−08 | B |

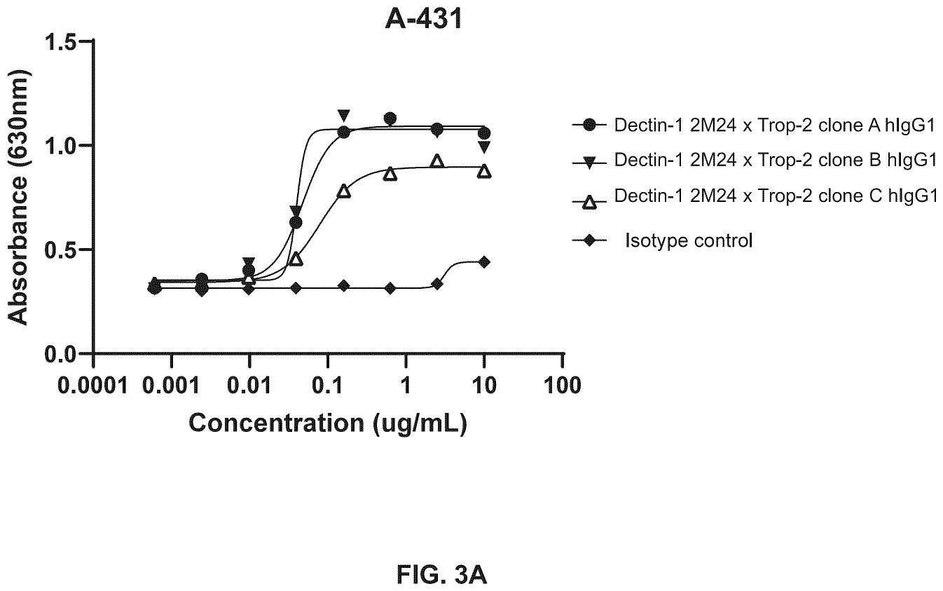

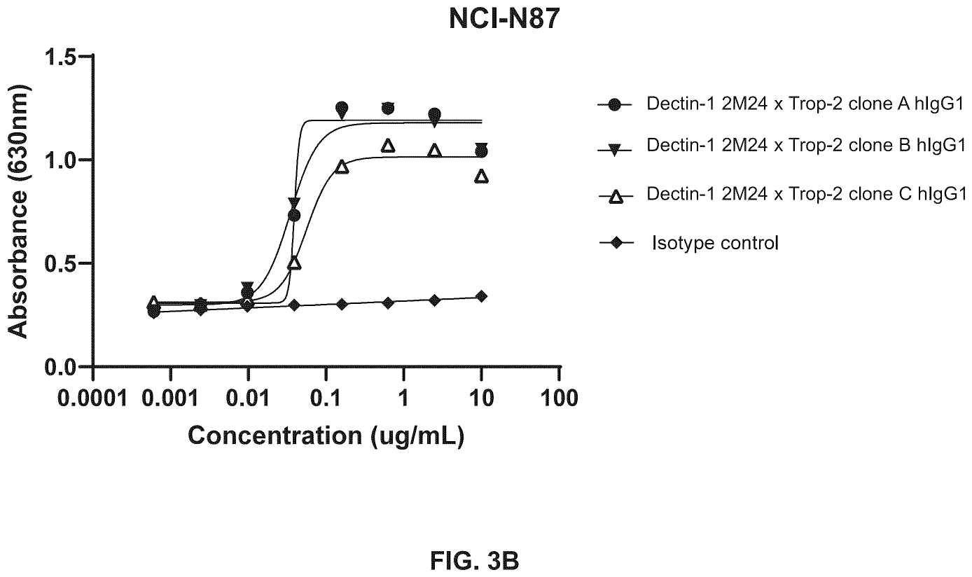

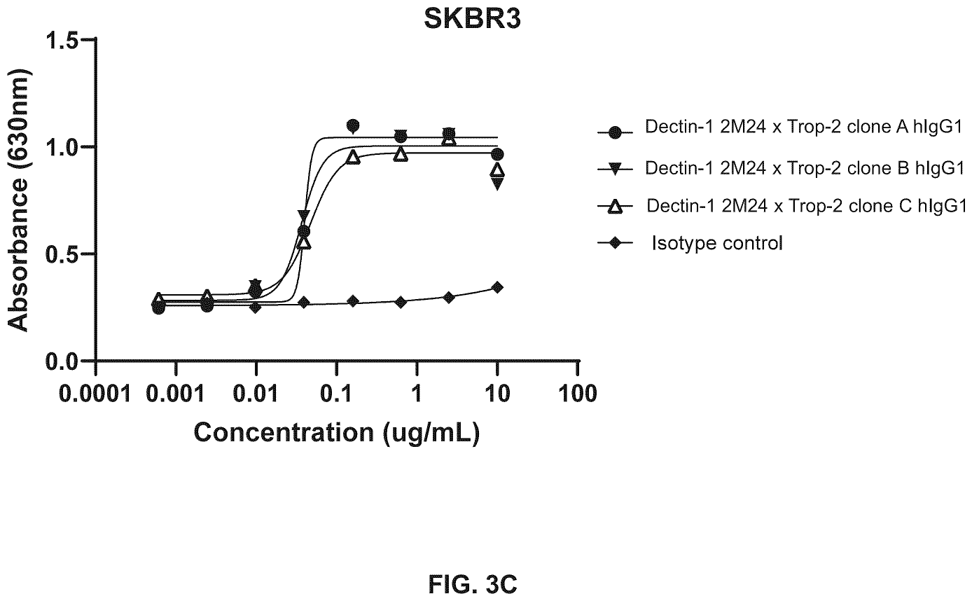

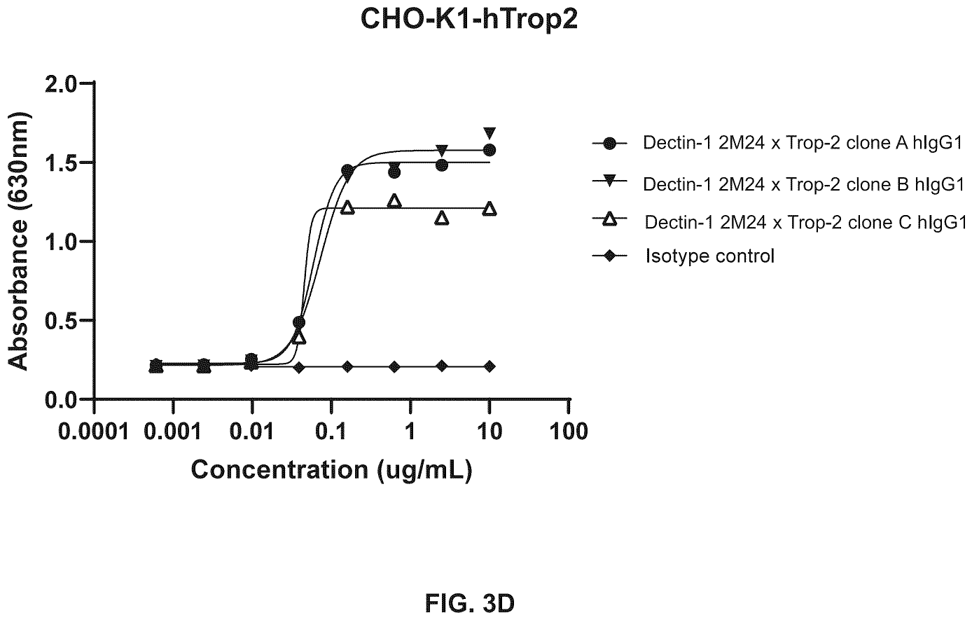

Ability of anti-Dectin-1 x anti-Trop-2 bispecific antibodies to activate the Dectin-1 pathway in the presence of cells expressing hTrop-2 was also measured. All three bispecific antibodies induced Dectin-1 pathway activation in the presence of Trop-2 expressing cell lines (FIGS. 3A-3D). EC50 values of each antibody in each cell line are shown in Table 3. SEAP secretion activity was found to correlate with binding data; bispecific antibody with Clone C anti-Trop-2 binding domain had the weakest EC50 and lowest Ymax.

TABLE 3
Dectin-1 pathway activation in the presence of indicated
hTrop-2-expressing cell lines (EC50, μg/mL).

| | | | | CHO-K1- |
|---|---|---|---|---|
| Antibody | A-431 | NCI-N87 | SKBR3 | hTrop2 |
| 2M24 × Trop-2 clone A hG1 | 0.05 | 0.04 | 0.04 | 0.06 |
| 2M24 × Trop-2 clone B hG1 | 0.04 | 0.03 | 0.04 | 0.07 |
| 2M24 × Trop-2 clone C hG1 | 0.08 | 0.06 | 0.05 | 0.05 |

Anti-Dectin-1 x anti-Trop-2 bispecific antibodies showed dose-dependent activation of NF-κB in presence of human cancer cells lines and CHO-K1-hTrop2 cells. The expression of Trop-2 in the cell lines used ranged from 50,000 to 400,000 copies, indicating that the anti-Dectin-1 x anti-Trop-2 bispecific antibodies can induce Dectin-1 clustering and activation of downstream NF-κB pathway in the presence of high or low Trop-2 expression levels.

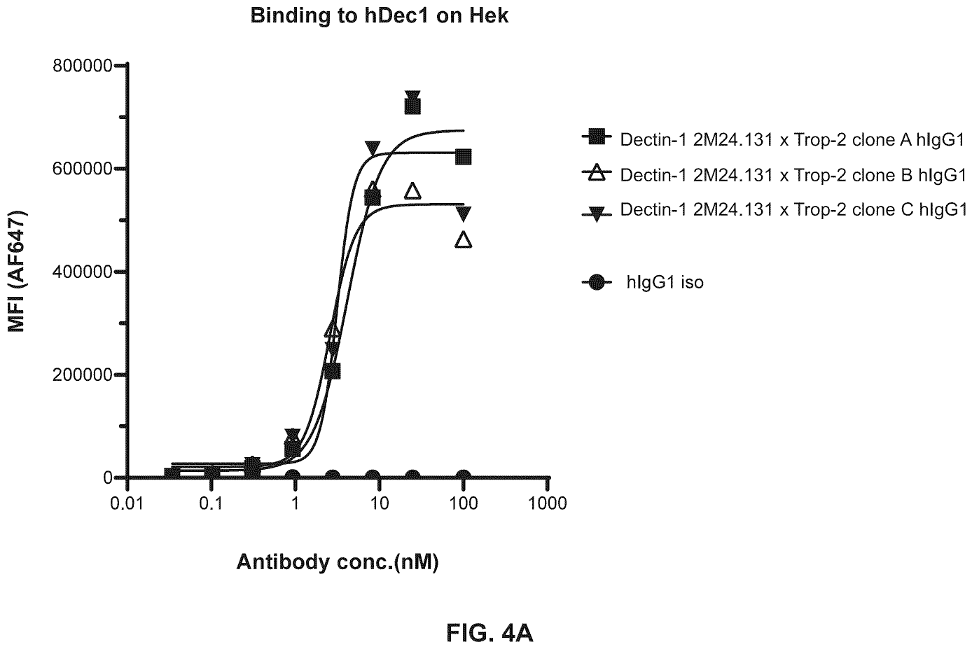

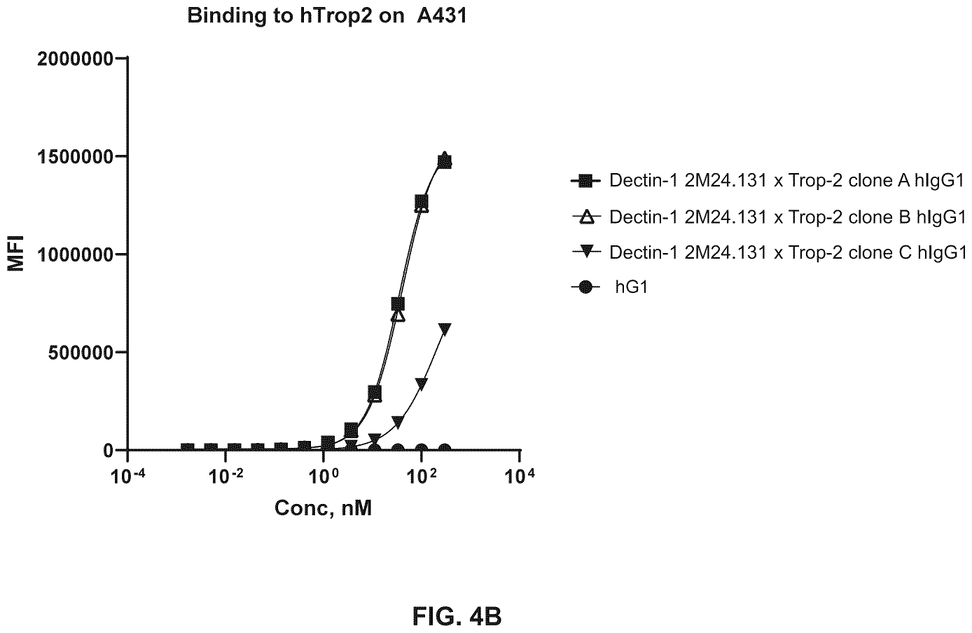

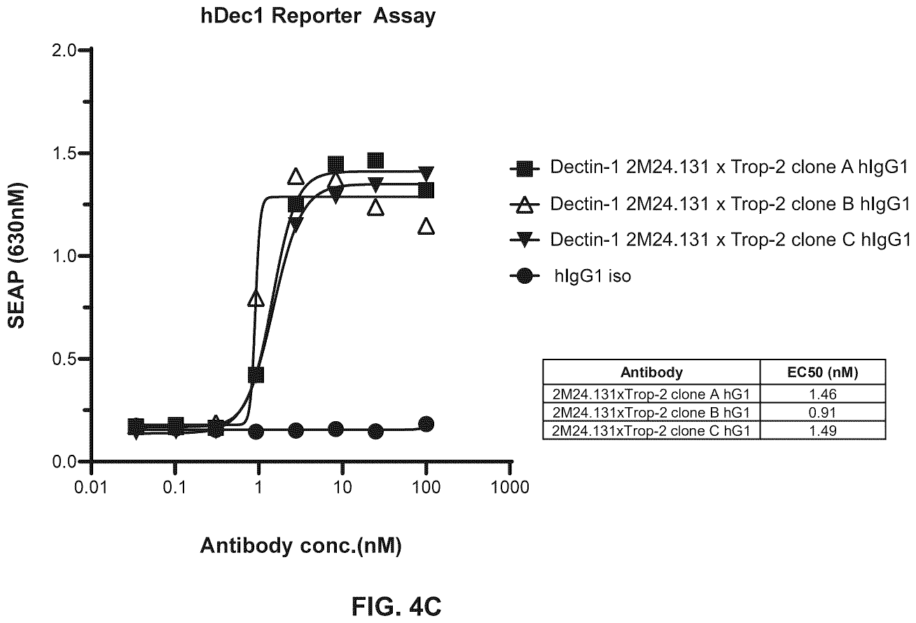

Anti-Dectin-1 x anti-Trop-2 bispecific antibodies were also tested in the format shown in FIG. 1C. Anti-Dectin-1 x anti-Trop-2 bispecific antibodies using the scFv format for the anti-Dectin-1 arm showed specific binding to cells expressing hDectin-1 (FIG. 4A) or hTrop-2 (FIG. 4B) comparable to the Fab format. Anti-Dectin-1 x anti-Trop-2 bispecific antibodies using the scFv format also induced Dectin-1 activation as measured by SEAP secretion (FIG. 4C). The use of an scFv-based Dectin-1 arm (as opposed to a traditional antibody Fab-based arm) is thought to be advantageous in manufacturing, as these bispecific antibodies have decreased dimer formation in purification and increased long-term stability. This specific anti-Dectin-1 antigen binding domain also demonstrates improved affinity and potentially less liability risk due to removal of certain amino acids compared to the parental 2M24 antigen binding domain.

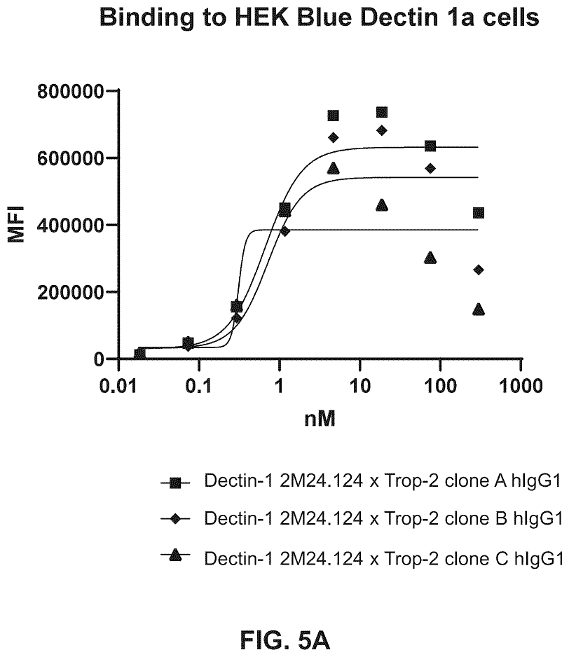

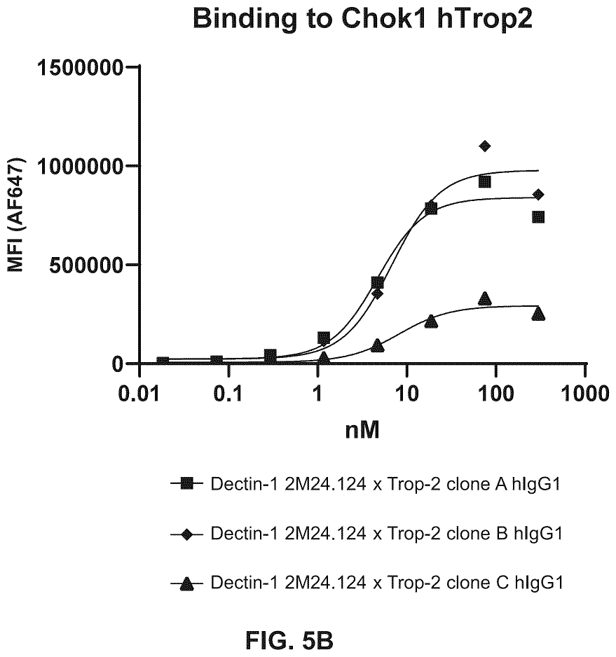

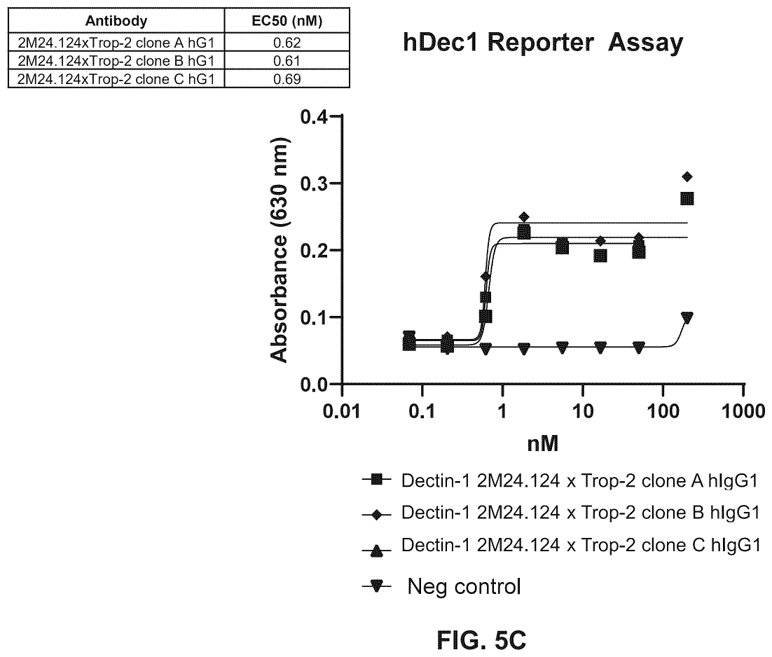

Anti-Dectin-1 x anti-Trop-2 bispecific antibodies were also tested in the format shown in FIG. 1B with engineered disulfides in the anti-Dectin-1 Fab arm. Anti-Dectin-1 x anti-Trop-2 bispecific antibodies using the Fab format with engineered disulfides in the anti-Dectin-1 arm showed specific binding to cells expressing h Dectin-1 (FIG. 5A) or hTrop-2 (FIG. 5B) comparable to the Fab format. Anti-Dectin-1 x anti-Trop-2 bispecific antibodies using the Fab format with engineered disulfides in the anti-Dectin-1 arm also induced Dectin-1 activation as measured by SEAP secretion (FIG. 5C). Advantageously, the use of an anti-Dectin-1 binding arm with engineered disulfides is thought to aid manufacturing by promoting proper heavy chain-light chain pairing during co-expression of the bispecific antibody arms. This specific anti-Dectin-1 antigen binding domain also demonstrates improved affinity and potentially less liability risk due to removal of certain amino acids compared to the parental 2M24 antigen binding domain.

In summary, all three formats provided anti-Dectin-1 x anti-Trop-2 bispecific antibodies that bind to each target with high affinity and induce Dectin-1 pathway activation.

### Example 2: Functional Characterization of Anti-Dectin-1 x Trop-2 Bispecific Antibodies

This example characterizes the functional activities of anti-Dectin-1 x anti-Trop-2 bispecific antibodies.

### Materials and Methods

### Phagocytosis

Flow cytometry analysis of co-cultures of human macrophages and calcein AM-labelled human cancer cells (3:1 effector:target ratio) in the presence of anti-Dectin-1 x anti-Trop-2 bispecific antibodies (with human IgG1 Fc) or isotype control. Co-cultures were incubated at 37° C. for 24 hours and then stained with a PE a-CD206 antibody to label macrophages. Phagocytosis was quantified as the percentage of FITC+ cancer cells engulfed by macrophages in the single gate. The primary antibodies were used in a serial dose titration. Bispecific antibody used the Fab format shown in FIGS. 1A & 1B. Anti-Dectin-1 binding arm comprised variable domains with the amino acid sequences of SEQ ID NO:24 for the VH domain and SEQ ID NO:25 for the VL domain. For the anti-Trop-2 binding arm, variable domains had the amino acid sequences of SEQ ID NO:69 for the VH domain and SEQ ID NO: 70 for the VL domain for Clone A, the amino acid sequences of SEQ ID NO: 74 for the VH domain and SEQ ID NO: 75 for the VL domain for Clone B, and the amino acid sequences of SEQ ID NO: 79 for the VH domain and SEQ ID NO: 80 for the VL domain for Clone C, respectively. All bispecific antibodies used a human IgG1 Fc region.

### Cytokine Release

PBMCs and CHO-K1-hTrop2 cells were mixed at 3:1 ratio in the presence of anti-Dectin-1 x anti-Trop-2 bispecific antibodies or isotype control. Co-cultures were incubated at 37 C for 24 hours and supernatants were collected for cytokine analysis by MSD (Meso Scale Discovery). Bispecific antibody used the Fab format shown in FIG. 1A. Anti-Dectin-1 binding arm comprised variable domains with the amino acid sequences of SEQ ID NO:24 for the VH domain and SEQ ID NO:25 for the VL domain. For the anti-Trop-2 binding arm, variable domains had the amino acid sequences of SEQ ID NO:69 for the VH domain and SEQ ID NO: 70 for the VL domain for Clone A, the amino acid sequences of SEQ ID NO: 74 for the VH domain and SEQ ID NO:75 for the VL domain for Clone B, and the amino acid sequences of SEQ ID NO: 79 for the VH domain and SEQ ID NO:80 for the VL domain for Clone C, respectively. All bispecific antibodies used a human IgG1 Fc region.

### Syngeneic Tumor Models

Eight-week-old female Balb/c mice or C57BL6 mice were inoculated subcutaneously with 2 million mouse colon cancer cells overexpressing human Trop2 (hTrop2-CT26) or 0.5 million mouse melanoma cancer cells overexpressing human Trop2 (hTrop2-B16F10), respectively. When tumors reached 80 mm³ the mice were randomized into 4 groups of 12 mice each and received six doses of anti-Dectin-1 x anti-Trop-2 bispecific antibody with anti-mDectin-1 Fab and anti-Trop-2 clone A, B, or C along with mIgG2a Fc (or isotype control) intraperitoneally at 10 mg/kg twice every week. The tumor volume and body weight were measured 2-3 times every week. Bispecific antibody used the Fab format shown in FIG. 1A. Anti-Dectin-1 binding arm used anti-mouse Dectin-1 variable domains. For the anti-Trop-2 binding arm, variable domains had the amino acid sequences of SEQ ID NO:69 for the VH domain and SEQ ID NO: 70 for the VL domain for Clone A, the amino acid sequences of SEQ ID NO:74 for the VH domain and SEQ ID NO: 75 for the VL domain for Clone B, and the amino acid sequences of SEQ ID NO: 79 for the VH domain and SEQ ID NO:80 for the VL domain for Clone C, respectively. All bispecific antibodies used a mouse IgG2a Fc region.

To test 2M24.119 anti-Dectin-1 binding arm directly, eight-week-old mixed gender C57BL6 mice expressing human Dectin1 were inoculated subcutaneously with 0.5 million of mouse melanoma cancer cells, overexpressing human Trop2 (hTrop2-B16F10). When tumors reached 80 mm³ the mice were randomized into groups of 12 mice each and received six doses of anti-Dectin-1 x anti-Trop-2 bispecific antibody with mIgG2a Fc (or isotype control) intraperitoneally at 10 mg/kg twice every week. The tumor volume and body weight were measured 2-3 times every week. Bispecific antibody used the Fab format shown in FIG. 1B. Anti-Dectin-1 binding arm used the amino acid sequences of SEQ ID NO: 30 for the VH domain and SEQ ID NO:31 for the VL domain. For the anti-Trop-2 binding arm, variable domains had the amino acid sequences of SEQ ID NO:69 for the VH domain and SEQ ID NO:70 for the VL domain for Clone A, the amino acid sequences of SEQ ID NO: 74 for the VH domain and SEQ ID NO: 75 for the VL domain for Clone B, and the amino acid sequences of SEQ ID NO:79 for the VH domain and SEQ ID NO:80 for the VL domain for Clone C, respectively. All bispecific antibodies used a mouse IgG2a Fc region.

### Cancer Cell Depletion in Human Tissue Biopsies

Human cancer biopsies were dissociated enzymatically, and single cell suspension was generated. 300,000 to 500,000 cells were treated with anti-Dectin-1 x anti-Trop-2 bispecific antibodies having human IgG1 Fc region at 5 μg/ml for 24 hours. Cancer cells were stained with APC-EPCAM antibody. Remaining cancer cells were assessed as single/live/CD45−/EPCAM+ cells and expressed as percentage of live cells.

### Results

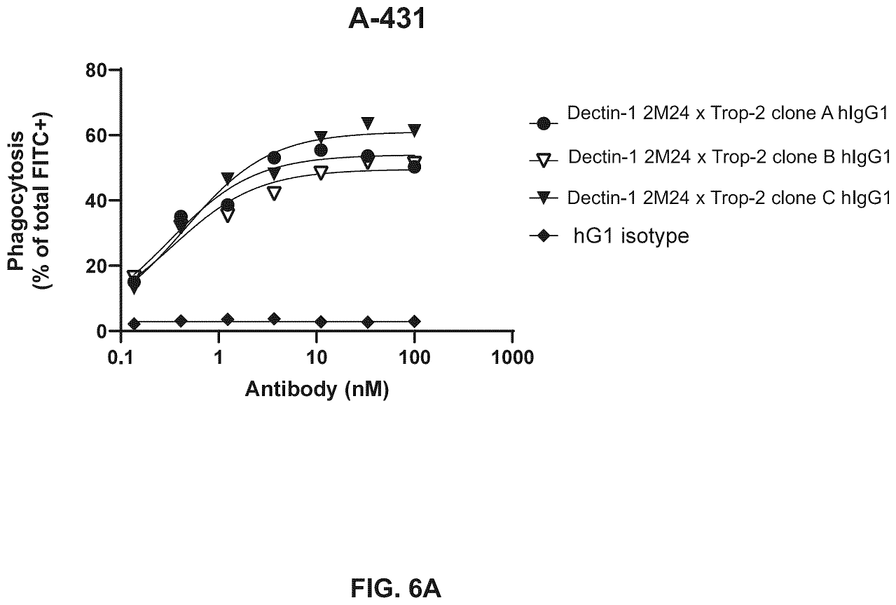

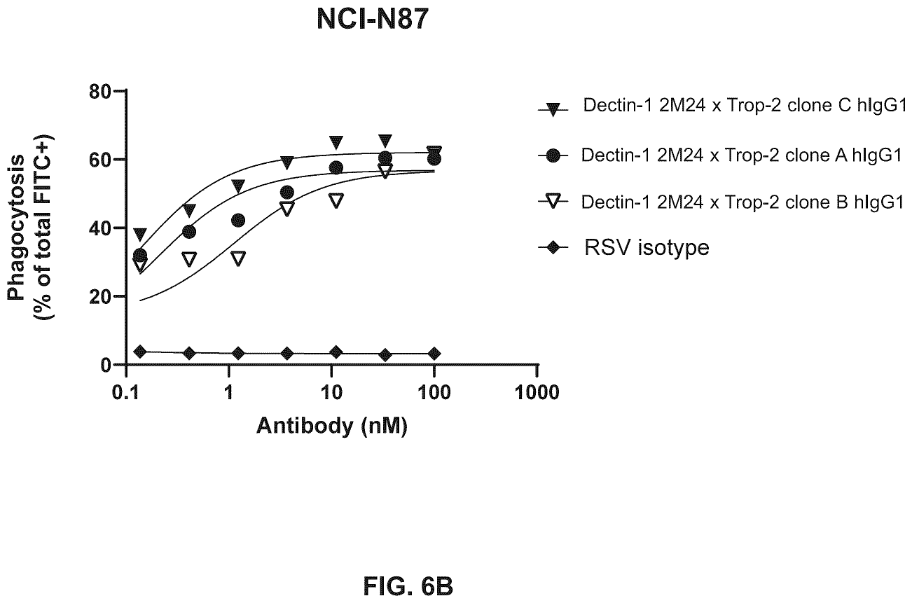

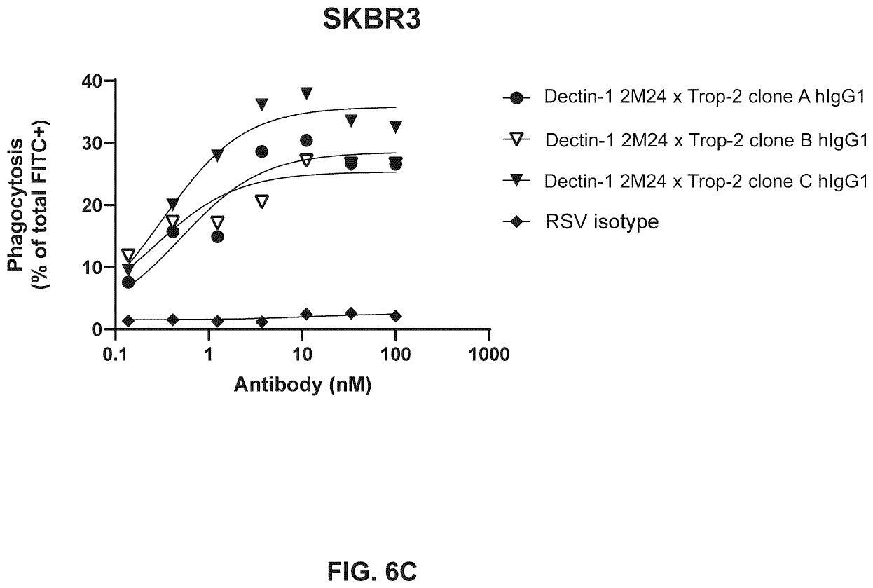

The ability of anti-Dectin-1 x anti-Trop-2 bispecific antibodies to induce phagocytosis of cancer cell lines by human macrophages was tested in vitro. All three anti-Dectin-1 x anti-Trop-2 bispecific antibodies were found to induce phagocytosis of A-431, NCI-N87, and SKBR3 cells (FIGS. 6A-6C). EC50 values of each antibody against each cell type are shown in Table 4.

TABLE 4
Induction of phagocytosis in vitro against indicated cell line by indicated
anti-Dectin-1 × anti-Trop-2 bispecific antibody (EC50, nM).

| Antibody | A-431 | NCI-N87 | SKBR3 |
|---|---|---|---|
| 2M24 × Trop-2 clone A hG1 | 0.31 | 0.1996 | 0.5075 |
| 2M24 × Trop-2 clone B hG1 | 0.34 | 1.124 | 0.2688 |
| 2M24 × Trop-2 clone C hG1 | 0.45 | 0.1321 | 0.3190 |

In summary, cancer cells were depleted in vitro by phagocytosis using the anti-Dectin-1 x anti-Trop-2 bispecific antibodies. The depletion was Fcγ receptor-mediated. These data support the power of the anti-Dectin-1 x anti-Trop-2 bispecific antibodies to promote the engagement and elimination of human cancer cells expressing Trop-2 by human macrophages.

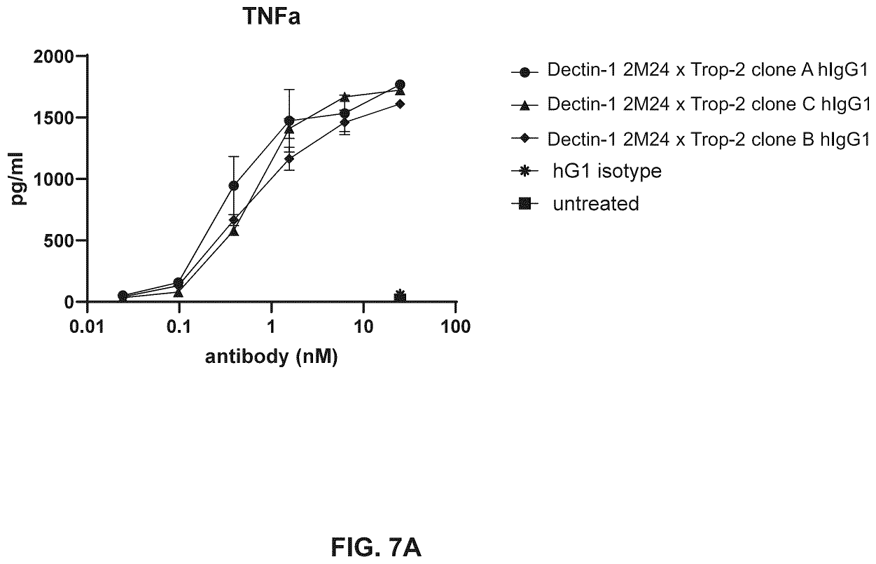

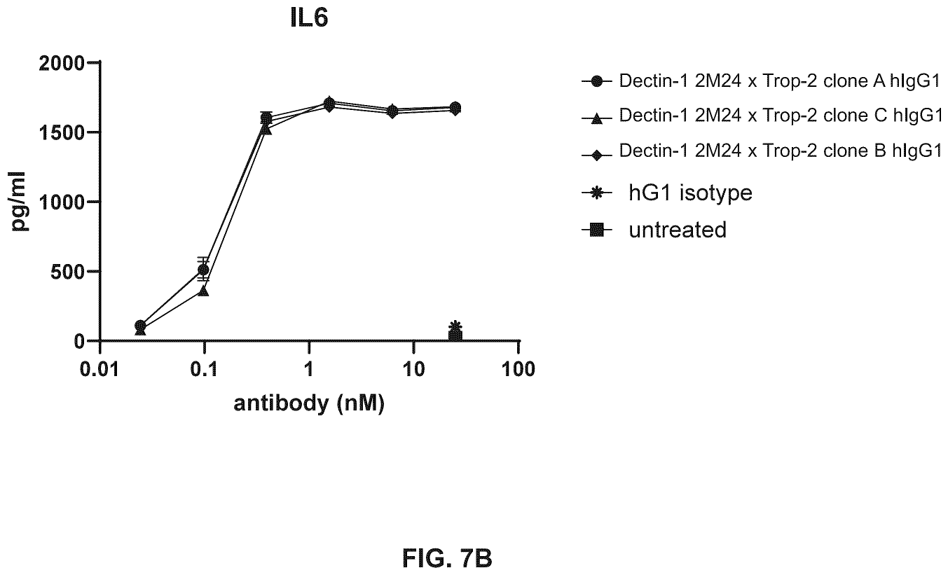

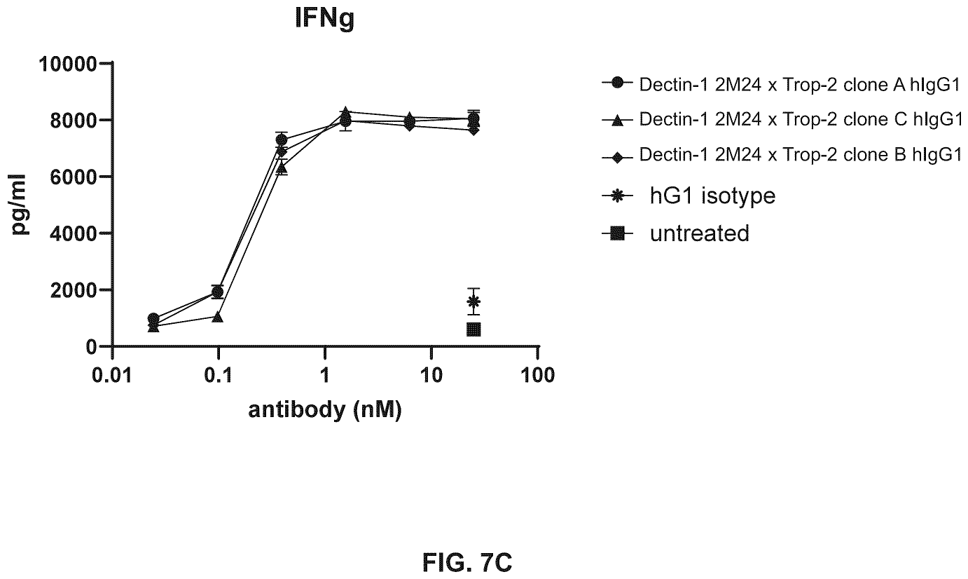

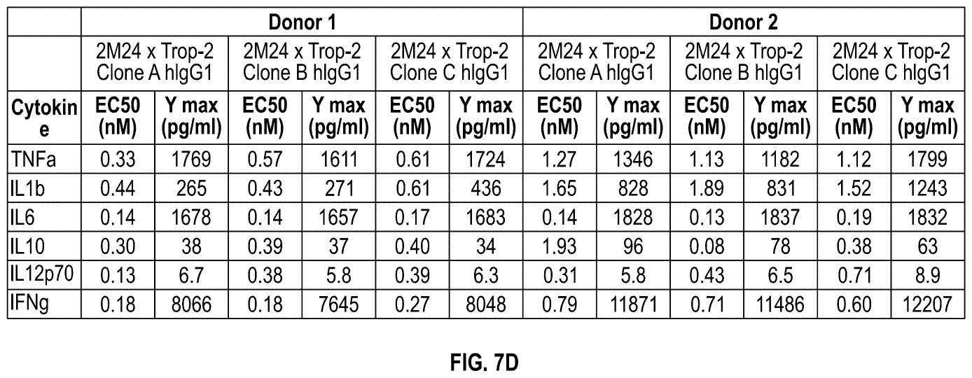

Cytokine secretion was also measured in co-cultures of human PBMCs and CHO-K1 cells expressing hTrop-2 treated with anti-Dectin-1 x anti-Trop-2 bispecific antibodies. As shown in FIGS. 7A-7D, cytokine secretion was not significantly affected by anti-Dectin-1 x anti-Trop-2 bispecific antibody.

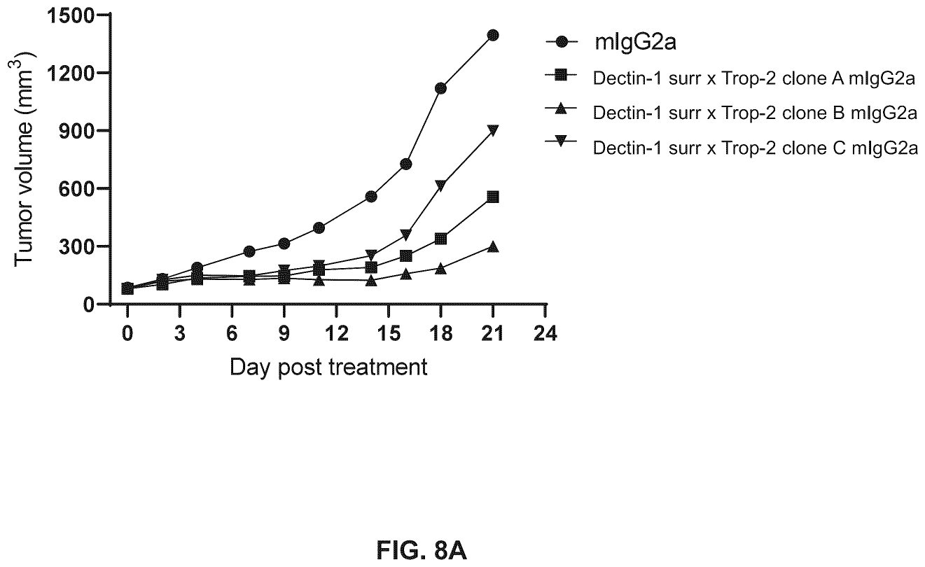

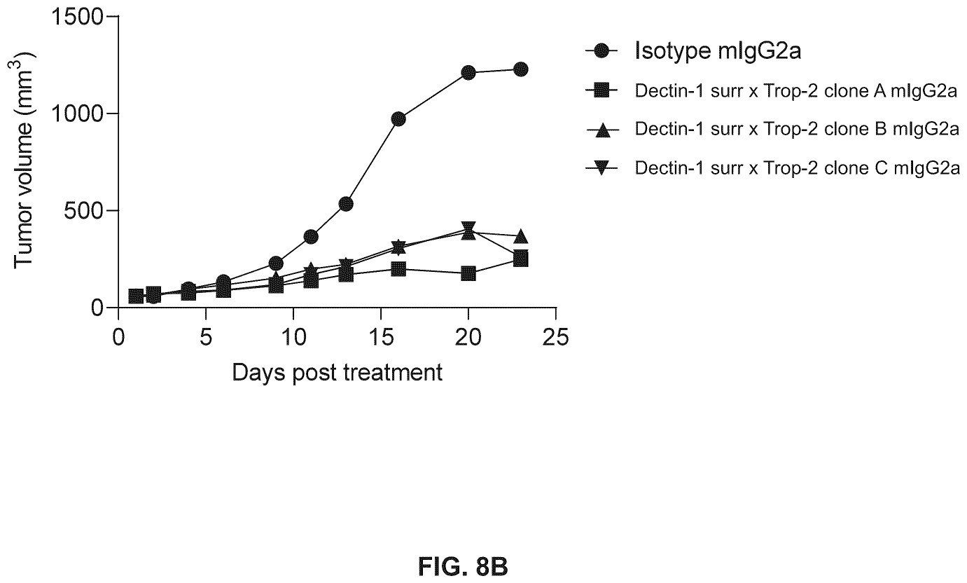

Anti-Dectin-1 x anti-Trop-2 bispecific antibodies were also tested in syngeneic mouse tumor models using surrogate anti-mDectin-1 binding arm and mIgG2a Fc. Anti-Dectin-1 x anti-Trop-2 bispecific antibodies were found to inhibit tumor growth in both colon cancer (FIG. 8A) and melanoma (FIG. 8B) models, showing significant efficacy in both preclinical models. Advantageously, the bispecific antibody with anti-Trop-2 clone B showed greatest anti-tumor activity. These models were thought to have fundamental differences in tumor microenvironment. CT26 tumors are considered “hot” with more infiltrated immune cells and generally responsive to immune modulators. In contrast. B16F10 are considered “cold” with less immune cell infiltration and not responsive to single agent immunotherapy. The efficacy produced in both models supports the versatility of anti-Dectin-1 x anti-Trop-2 bispecific antibodies to work in both “hot” and harder to treat “cold” tumors, deriving by their ability to modulate the tumor microenvironment.

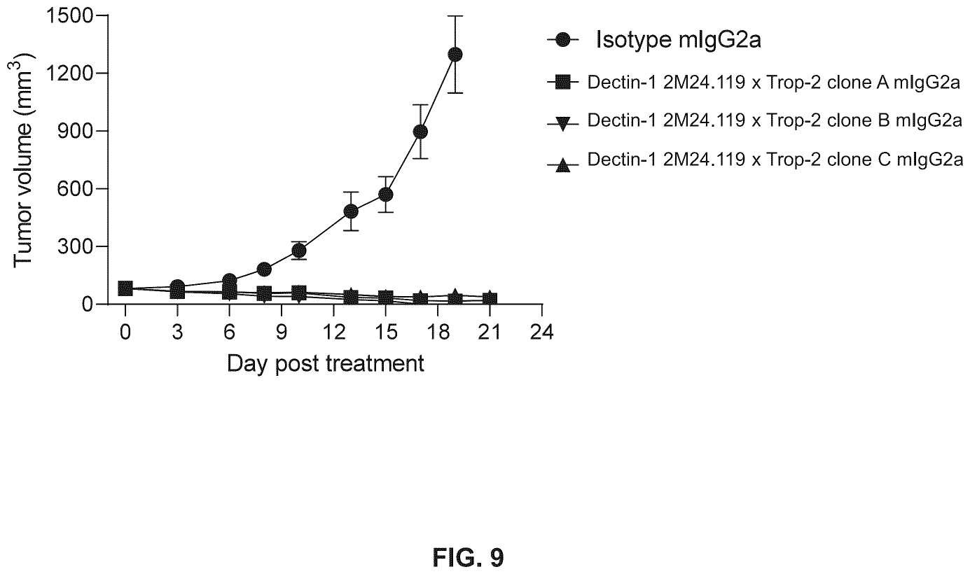

Anti-Dectin-1 x anti-Trop-2 bispecific antibodies were also tested in syngeneic mouse B16F10 tumor model using the disulfide-engineered anti-Dectin-1 variable domains in mice expressing human Dectin-1. The anti-Dectin-1 x anti-Trop-2 bispecific antibodies showed significant anti-tumor efficacy in this melanoma model (FIG. 9). This study directly demonstrates the ability of anti-Dectin-1 x anti-Trop-2 bispecific antibodies to confer anti-tumor effects by engaging human Dectin-1, supporting the use of anti-Dectin-1 x anti-Trop-2 bispecific antibodies in oncology.

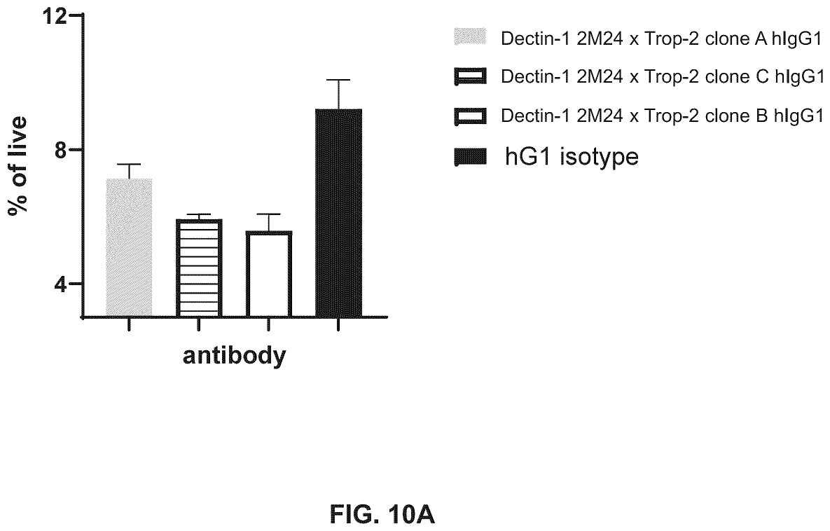

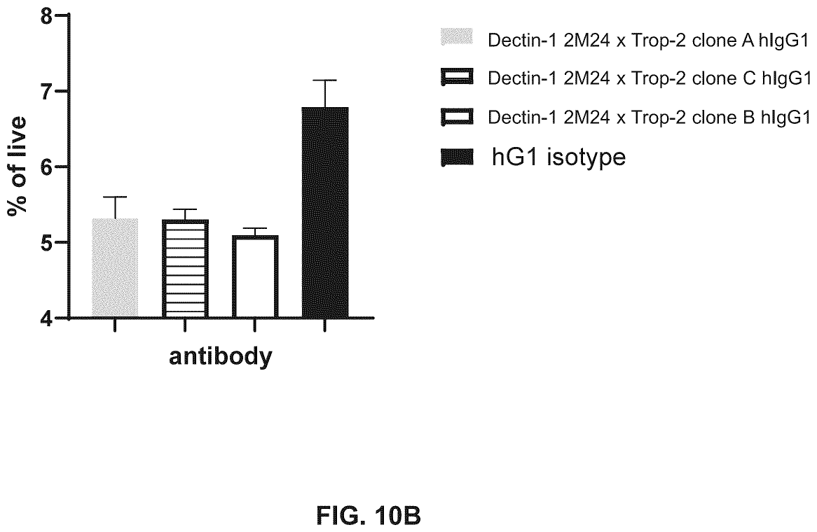

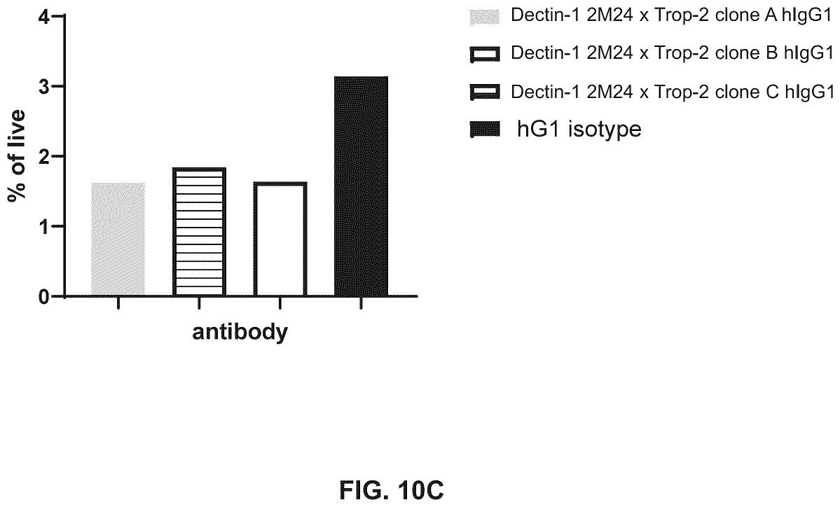

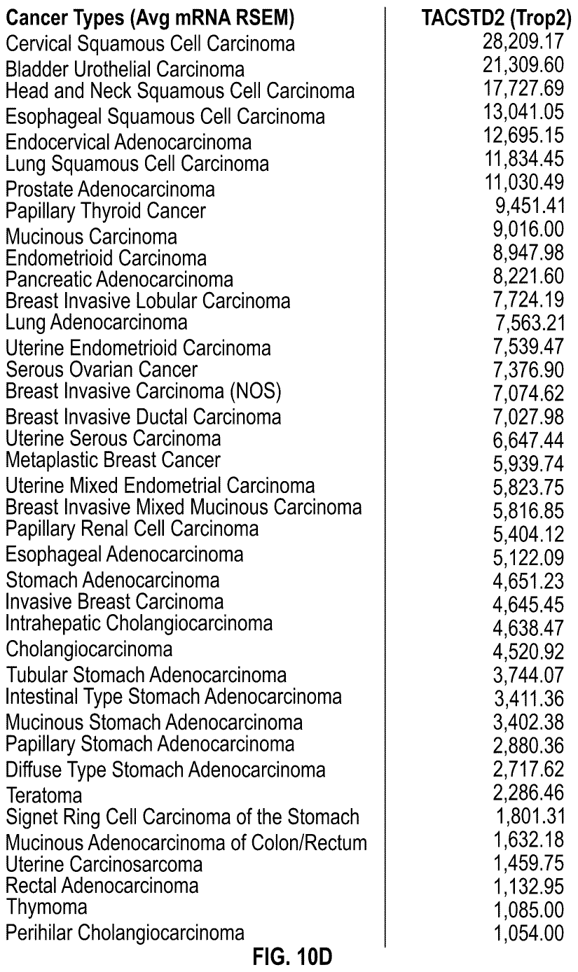

Anti-Dectin-1 x anti-Trop-2 bispecific antibodies were also tested for ability to deplete single cell suspensions derived from tumor biopsies. Anti-Dectin-1 x anti-Trop-2 bispecific antibodies were found to induce depletion of tumor cells from human lung (FIG. 10A), ovarian (FIG. 10B), and uterine (FIG. 10C) cancer biopsies. Anti-Dectin-1 x anti-Trop-2 bispecific antibodies achieved a 20-50% reduction of cancer cells expressing Trop-2 in various human cancer biopsies. Advantageously, the bispecific antibody with anti-Trop-2 clone B showed improved activity.

These data indicate that the bispecific antibodies can induce the phagocytosis and elimination of cancer cells by the tumor associated macrophages within solid cancer biopsies, indicating activity in an endogenous cancer tissue environment. As Trop-2 is widely expressed in human cancers as assayed by mRNA expression level (FIG. 10D), this strategy may be broadly applicable in solid tumors.

Although the present disclosure has been described in some detail by way of illustration and example for purposes of clarity of understanding, the descriptions and examples should not be construed as limiting the scope of the present disclosure. The disclosures of all patent and scientific literature cited herein are expressly incorporated in the entirety by reference.

1-63. (canceled)

64. A bispecific binding molecule, comprising:
(a) a first arm comprising a first antigen binding domain that binds to human Dectin-1; and
(b) a second arm comprising a second antigen binding domain that binds to human Trop-2;
wherein the second antigen binding domain comprises a second heavy chain variable (VH) domain and a second light chain variable (VL) domain; wherein the second VH domain comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and wherein the second VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO: 42.

65. The bispecific binding molecule of claim 64, wherein the second VH domain comprises the amino acid sequence of SEQ ID NO: 74, and the second VL domain comprises the amino acid sequence of SEQ ID NO:75.

66. The bispecific binding molecule of claim 64, wherein the second arm comprises:
(a) an antibody heavy chain polypeptide that comprises the second VH domain and an Fc region, and
(b) an antibody light chain polypeptide that comprises the second VL domain.

67. The bispecific binding molecule of claim 64, wherein the second arm comprises an antibody heavy chain polypeptide that comprises the amino acid sequence of SEQ ID NO:76 or 77 and an antibody light chain polypeptide that comprises the amino acid sequence of SEQ ID NO: 78.

68. The bispecific binding molecule of claim 64, wherein the first antigen binding domain comprises a first heavy chain variable (VH) domain and a first light chain variable (VL) domain; wherein the first VH domain comprises a CDR-H1 comprising the amino acid sequence of SEQ ID NO:16, a CDR-H2 comprising the amino acid sequence of SEQ ID NO: 17, and a CDR-H3 comprising the amino acid sequence of SEQ ID NO: 18; and wherein the first VL domain comprises a CDR-L1 comprising the amino acid sequence of SEQ ID NO:4, a CDR-L2 comprising the amino acid sequence of SEQ ID NO:19, and a CDR-L3 comprising the amino acid sequence of SEQ ID NO:20.

69. The bispecific binding molecule of claim 65, wherein the first VH domain comprises the amino acid sequence of SEQ ID NO:30, and the first VL domain comprises the amino acid sequence of SEQ ID NO:31.

70. The bispecific binding molecule of claim 68, wherein the first VH domain comprises the amino acid sequence of SEQ ID NO:30, and the first VL domain comprises the amino acid sequence of SEQ ID NO:31.

71. The bispecific binding molecule of claim 64, wherein the first antigen binding domain comprises a first heavy chain variable (VH) domain and a first light chain variable (VL) domain; wherein the first VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; and wherein the first VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6.

72. The bispecific binding molecule of claim 65, wherein the first VH domain comprises the amino acid sequence of SEQ ID NO:24, and the first VL domain comprises the amino acid sequence of SEQ ID NO:25.

73. The bispecific binding molecule of claim 71, wherein the first VH domain comprises the amino acid sequence of SEQ ID NO:24, and the first VL domain comprises the amino acid sequence of SEQ ID NO:25.

74. The bispecific binding molecule of claim 64, wherein the first arm comprises:
(a) an antibody heavy chain polypeptide that comprises the first VH domain and an Fc region, and
(b) an antibody light chain polypeptide that comprises the first VL domain.

75. The bispecific binding molecule of claim 64, wherein the first arm comprises an antibody heavy chain polypeptide that comprises the amino acid sequence of SEQ ID NO:32 or 91 and an antibody light chain polypeptide that comprises the amino acid sequence of SEQ ID NO: 33.

76. The bispecific binding molecule of claim 64, wherein the first arm comprises an antibody heavy chain polypeptide that comprises the amino acid sequence of SEQ ID NO:87 or 88 and an antibody light chain polypeptide that comprises the amino acid sequence of SEQ ID NO: 89.

77. The bispecific binding molecule of claim 64, wherein the first arm comprises an antibody heavy chain polypeptide that comprises the first VH domain and an Fc region, and an antibody light chain polypeptide that comprises the first VL domain; wherein the second arm comprises an antibody heavy chain polypeptide that comprises the second VH domain and an Fc region, and an antibody light chain polypeptide that comprises the second VL domain; and wherein the Fc regions of the first and second arms are human IgG1 or human IgG4 Fc regions.

78. The bispecific binding molecule of claim 77, wherein the Fc region of the first arm comprises one or more knob-forming mutations, and the Fc region of the second arm comprises one or more cognate hole-forming mutations; or wherein the Fc region of the second arm comprises one or more knob-forming mutations, and the Fc region of the first arm comprises one or more cognate hole-forming mutations.

79. The bispecific binding molecule of claim 78, wherein one or more knob-forming mutations comprise a T366W substitution, and wherein the one or more cognate hole-forming mutations comprise T366S, L368A, and Y407V substitutions, according to EU numbering.

80. A bispecific binding molecule, comprising:
(a) a first arm comprising a first antigen binding domain that binds to human Dectin-1; and
(b) a second arm comprising a second antigen binding domain that binds to human Trop-2;
wherein the first arm comprises a first antibody heavy chain polypeptide and a first antibody light chain polypeptide, and the second arm comprises a second antibody heavy chain polypeptide and a second antibody light chain polypeptide; and
wherein the first antibody heavy chain polypeptide comprises the amino acid sequence of SEQ ID NO:32 or 91, the first antibody light chain polypeptide comprises the amino acid sequence of SEQ ID NO:33, the second antibody heavy chain polypeptide comprises the amino acid sequence of SEQ ID NO:76 or 77, and the second antibody light chain polypeptide comprises the amino acid sequence of SEQ ID NO: 78.

81. A polynucleotide encoding the bispecific binding molecule of claim 64.

82. A vector comprising the polynucleotide of claim 81.

83. An isolated host cell comprising the polynucleotide of claim 81.

84. A method of producing a bispecific binding molecule, comprising culturing the host cell of claim 83 under conditions suitable for production of the bispecific binding molecule.

85. A pharmaceutical composition comprising the bispecific binding molecule of claim 64 and a pharmaceutically acceptable carrier.

86. A pharmaceutical composition comprising the bispecific binding molecule of claim 80 and a pharmaceutically acceptable carrier.

87. A method of treating cancer, comprising administering an effective amount of the pharmaceutical composition of claim 85 to an individual in need thereof.

88. A method of treating cancer, comprising administering an effective amount of the pharmaceutical composition of claim 86 to an individual in need thereof.

89. The method of claim 88, wherein the individual is a human, wherein cells of the cancer express human Trop-2, and wherein the cancer is a solid cancer.

90. A binding molecule comprising an antigen binding domain that binds to human Trop-2 (anti-Trop-2 antigen binding domain), wherein the anti-Trop-2 antigen binding domain comprises a heavy chain variable (VH) domain and a light chain variable (VL) domain, and wherein:
(a) the VH domain comprises a CDR-H1 comprising the amino acid sequence of SEQ ID NO: 37, a CDR-H2 comprising the amino acid sequence of SEQ ID NO:38, and a CDR-H3 comprising the amino acid sequence of SEQ ID NO:39, and the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO: 42; or
(b) the VH domain comprises a CDR-H1 comprising the amino acid sequence of SEQ ID NO: 53, a CDR-H2 comprising the amino acid sequence of SEQ ID NO:54, and a CDR-H3 comprising the amino acid sequence of SEQ ID NO:55, and the VL domain comprises a CDR-L1 comprising the amino acid sequence of SEQ ID NO:56, a CDR-L2 comprising the amino acid sequence of SEQ ID NO:57, and a CDR-L3 comprising the amino acid sequence of SEQ ID NO: 58.

91. The binding molecule of claim 90, wherein the binding molecule is a multispecific binding molecule.
